SEQUENCE LISTING (8 sequences)

SEQ ID NO: 1 - protein, 450 aa, synthetic construct
  REGION 1..450: note = Muromonab heavy chain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, synthetic construct
  REGION 1..213: note = Muromonab light chain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 134 aa, synthetic construct
  REGION 1..134: note = recombinant human IL-2 (rhIL-2)
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - protein, 132 aa, synthetic construct
  REGION 1..132: note = Aldesleukin
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 130 aa, synthetic construct
  REGION 1..130: note = recombinant human IL-4 (rhIL-4)
MHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 6 - protein, 153 aa, synthetic construct
  REGION 1..153: note = recombinant human IL-7 (rhIL-7)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 7 - protein, 115 aa, synthetic construct
  REGION 1..115: note = recombinant human IL-15 (rhIL-15)
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - protein, 132 aa, synthetic construct
  REGION 1..132: note = recombinant human IL-21 (rhIL-21)
MQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

(19) United States
(12) Patent Application Publication
(10) Pub. No.: US 2023/0165898 A1
(43) Pub. Date: Jun. 1, 2023

(54) EXPANSION OF TUMOR INFILTRATING LYMPHOCYTES FROM LIQUID TUMORS AND THERAPEUTIC USES THEROF

(71) Applicant: Iovance Biotherapeutics, Inc., San Carlos, CA (US)

(72) Inventor: Lavakumar Karyampudi, Tampa, FL (US)

(21) Appl. No.: 17/812,146

(22) Filed: Jul. 12, 2022

Related U.S. Application Data
(63) Continuation of application No. 17/434,499, filed on Aug. 27, 2021.
(60) Provisional application No. 62/857,219, filed on Jun. 4, 2019.
(60) Provisional application No. 62/812,900, filed on Mar. 1, 2019.

(51) Int. Cl. A61K 35/17; C12N 5/0783

(52) U.S. Cl. CPC A61K 35/17; C12N 5/0638; C12N 5/0081

(57) ABSTRACT

Methods of expanding peripheral blood lymphocytes (PBLs) from blood of patients with hematological malignancies, including lymphomas and leukemias, genetic modifications of expanded PBLs to incorporate chimeric antigen receptors, genetically modified T cell receptors, and other genetic modifications, and uses of such expanded and/or modified PBLs in the treatment of diseases such as cancers and hematological malignancies are disclosed herein.

### CROSS REFERENCE TO RELATED APPLICATIONS

This application is a continuation of U.S. application Ser. No. 17/434,499, filed Aug. 27, 2021, which is a U.S. National Stage application of International Application No. PCT/US2020/020505, filed on Feb. 28, 2020, which claims priority to U.S. Provisional Application No. 62/812,900, filed on Mar. 1, 2019 and U.S. Provisional Application No. 62/857,219, filed on Jun. 4, 2019, each of which is herein incorporated by reference in its entirety.

### FIELD OF THE INVENTION

Methods of expanding peripheral blood lymphocytes (PBLs) derived from blood and/or bone marrow of a patient with a hematological malignancy, such as a liquid tumor, including lymphomas and leukemias, and compositions comprising populations of PBLs obtained therefrom, are disclosed herein. In addition, therapeutic uses of autologous PBLs expanded from blood of a patient in the treatment of hematological malignancies are disclosed herein.

### BACKGROUND OF THE INVENTION

Treatment of bulky, refractory cancers using adoptive autologous transfer of tumor infiltrating lymphocytes (TILs) represents a powerful approach to therapy for patients with poor prognoses. Gattinoni, et al., Nat. Rev. Immunol. 2006, 6, 383-393. TILs are dominated by T cells, and IL-2-based TIL expansion followed by a “rapid expansion process” (REP) has become a preferred method for TIL expansion because of its speed and efficiency. Dudley, et al., Science 2002, 298, 850-54; Dudley, et al., J. Clin. Oncol. 2005, 23, 2346-57; Dudley, et al., J. Clin. Oncol. 2008, 26, 5233-39; Riddell, et al., Science 1992, 257, 238-41; Dudley, et al., J. Immunother. 2003, 26, 332-42. A number of approaches to improve responses to TIL therapy in melanoma and to expand TIL therapy to other tumor types have been explored with limited success, and the field remains challenging. Goff, et al., J. Clin. Oncol. 2016, 34, 2389-97; Dudley, et al., J. Clin. Oncol. 2008, 26, 5233-39; Rosenberg, et al., Clin. Cancer Res. 2011, 17, 4550-57. Earlier approaches to expansions of TILs from B cell lymphomas yielded poor results, with only 2 of 12 attempts at TIL growth providing for potential activity against tumors. Schwartzentruber, et al., Blood 1993, 82, 1204-1211. There is an urgent need to provide for more efficacious therapies in many hematological malignancies, including chronic lymphocytic leukemia (CLL). There is also an urgent need to provide such therapies using whole blood as a source of lymphocytes with TIL functionality, such as PBLs, to treat patients refractory to other therapies or that have relapsed. Because of the burden of apheresis and the large blood volumes taken from critically ill cancer patients, there is also an urgent need to use as little as patient blood as possible.

The present invention provides the surprising finding that PBLs expansion processes using low volumes of blood as a source of PBLs can result in efficacious PBL populations obtained from hematological malignancies, such as liquid tumors, including lymphomas or leukemias.

### SUMMARY OF THE INVENTION

In an embodiment of the invention, a method for expanding peripheral blood lymphocytes (PBLs) from peripheral blood is disclosed. In one embodiment, the method comprises (a) obtaining a sample of peripheral blood mononuclear cells (PBMCs) from the peripheral blood of a patient, wherein said sample is optionally cryopreserved and the patient is optionally pretreated with an ITK inhibitor; (b) optionally washing the PBMCs by centrifugation; (c) admixing magnetic beads selective for CD3 and CD28 to the PBMCs to form an admixture of the beads and the PBMCs; (d) seeding the admixture of the beads and the PBMCs into a gas-permeable container and co-culturing said PBMCs in media comprising about 3000 IU/mL of IL-2 in for about 4 to about 6 days; (e) feeding said PBMCs using media comprising about 3000 IU/mL of IL-2, and co-culturing said PBMCs for about 5 days, such that the total co-culture period of steps (d) and (e) is about 9 to about 11 days; (f) harvesting PBMCs from media; (g) removing the magnetic beads selective for CD3 and CD28 using a magnet; (h) removing residual B-cells using magnetic-activated cell sorting and beads selective for CD19 to provide a PBL product; (i) washing and concentrating the PBL product using a cell harvester; and (j) formulating and optionally cryopreserving the PBL product. In one embodiment, the ITK inhibitor is optionally an ITK inhibitor that covalently binds to ITK.

In one embodiment, the method comprises (a) obtaining a sample of peripheral blood mononuclear cells (PBMCs) from the peripheral blood of a patient, wherein said sample is optionally cryopreserved and the patient is optionally pretreated with an ITK inhibitor; (b) optionally washing the PBMCs by centrifugation; (c) removing B-cells from the PBMCs by selecting against CD19 to provide PBMCs depleted of B-cells; (d) admixing magnetic beads selective for CD3 and CD28 to the PBMCs depleted of B-cells to form an admixture of the beads and the PBMCs; (e) seeding the admixture of the beads and the PBMCs into a gas-permeable container and co-culturing said PBMCs in media comprising about 3000 IU/mL of IL-2 in for about 4 to about 6 days; (f) feeding said PBMCs using media comprising about 3000 IU/mL of IL-2, and co-culturing said PBMCs for about 5 days, such that the total co-culture period of steps (e) and (f) is about 9 to about 11 days; (g) harvesting the PBMCs from media; (h) removing any residual magnetic beads selective for CD3 and CD28 from the PBMCs using a magnet to provide a PBL product; (i) washing and concentrating the PBL product using a cell harvester; and (j) formulating and optionally cryopreserving the PBL product. In one embodiment, the ITK inhibitor is optionally an ITK inhibitor that covalently binds to ITK. In another embodiment, the removal of B-cells in step (c) is performed by using beads selective for CD19 to remove B-cells from the PBMCs. In another embodiment, the removal of B-cells in step (c) is performed by admixing the beads selective for CD19 with the PBMCs to form complexes of beads and B-cells in an admixture with the PBMCs and removing the complexes from the admixture. In another embodiment, the removal of B-cells in step (c) is performed by admixing magnetic beads selective for CD19 with the PBMCs to form complexes of magnetic beads and B-cells in the admixture and using a magnet to remove the complexes from the admixture. In an embodiment of the invention, the beads selective for CD19 are beads conjugated to anti-CD19 antibody.

In an embodiment of the invention, the amount of peripheral blood that is obtained from a patient in a method according to the present invention is between about 10 mL and 50 mL. In another embodiment, the amount of peripheral blood that is obtained from a patient is less than or equal to about 50 mL.

In an embodiment of the invention, the seeding density of the PBMCs in a method according to the present invention is about 2×10⁵/cm² to about 1.6×10³/cm² relative to the surface area of the gas-permeable container.

In an embodiment of the invention, a process for the preparation of peripheral blood lymphocytes (PBLs) from a whole blood sample comprises the steps of (a) obtaining peripheral blood mononuclear cells (PBMCs) from less than or equal to about 50 mL of whole blood from a patient having a liquid tumor, wherein the patient is optionally pretreated with an ITK inhibitor; (b) admixing beads selective for CD3 and CD28 with the PBMCs, wherein the beads are added at a ratio of 3 beads:1 cell, to form an admixture of the PBMCs and the beads; (c) culturing the admixture of the PBMCs and the beads at a density of about 25,000 cells per cm² to about 50,000 cells per cm² on a gas-permeable surface of one or more containers containing a first cell culture medium and IL-2 for a period of about 4 days; (d) adding to each container IL-2 and a second cell culture medium that is the same as or different from the first cell culture medium and culturing for a period of about 5 days to about 7 days to form an expanded population of PBLs; and (e) harvesting from each container the expanded population of PBLs.

In an embodiment of the invention, a process for the preparation of peripheral blood lymphocytes (PBLs) from a whole blood sample comprises the steps of (a) obtaining peripheral blood mononuclear cells (PBMCs) from less than or equal to about 50 mL of whole blood from a patient having a liquid tumor, wherein the patient is optionally pretreated with an ITK inhibitor; (b) removing B-cells from the PBMCs by selecting against CD19 to provide PBMCs depleted of B-cells; (c) admixing beads selective for CD3 and CD28 to the PBMCs, wherein the beads are added at a ratio of 3 beads:1 cell, to form an admixture of the PBMCs and the beads; (d) culturing the admixture of the PBMCs and the beads at a density of about 25,000 cells per cm² to about 50,000 cells per cm² on a gas-permeable surface of one or more containers containing a first cell culture medium and IL-2 for a period of about 4 days; (e) adding to each container IL-2 and a second cell culture medium that is the same as or different from the first cell culture medium and culturing for a period of about 5 days to about 7 days to form an expanded population of PBLs; and (f) harvesting from each container the expanded population of PBLs. In one embodiment, the ITK inhibitor is optionally an ITK inhibitor that covalently binds to ITK. In another embodiment, the patient is pretreated with an ITK inhibitor and the patient is refractory to treatment with the ITK inhibitor. In another embodiment, the removal of B-cells in step (b) is performed by using beads selective for CD19 to remove B-cells from the PBMCs. In another embodiment, the removal of B-cells in step (b) is performed by admixing the beads selective for CD19 with the PBMCs to form complexes of the beads and B-cells in an admixture with the PBMCs and removing the complexes from the admixture. In another embodiment, the removal of B-cells is performed by admixing magnetic beads selective for CD19 to the PBMCs to form complexes of the magnetic beads and B-cells in an admixture with the PBMCs and using a magnet to remove the complexes from the admixture. In another embodiment, the beads selective for CD19 are beads conjugated to anti-CD19 antibody.

In an embodiment of the method according to the present invention, the total number of cells harvested is from about 8 billion to about 22 billion.

In an embodiment of the method according to the present invention, the total number of cells harvested is from about 1 billion to about 8 billion.

In an embodiment of the method according to the present invention, about 95% to about 99% of the cells harvested are T-cells.

In an embodiment of the method according to the present invention, the step of admixing the beads selective for CD3 and CD28 to the PBMCs to form an admixture of the beads and the PBMCs is replaced with the step of admixing the beads selective for CD3 and CD28 to the PBMCs to form complexes of the beads and the PBMCs in an admixture of the beads and the PBMCs, and the step of culturing the admixture is replaced with the step of separating the complexes of the beads and the PBMCs from the admixture and culturing the complexes of the PBMCs and the beads at a density of about 25,000 cells per cm² to about 50,000 cells per cm² on a gas-permeable surface in one or more containers containing a first cell culture medium and IL-2 for a period of about 4 days. In another embodiment of the present invention, the beads selective for CD3 and CD28 are magnetic beads, and the step of separating the complexes of the beads and the PBMCs from the admixture is performed by using a magnet to remove the complexes from the admixture.

In an embodiment of the invention, the beads selective for CD3 and CD28 are beads conjugated to anti-CD3 antibodies and anti-CD28 antibodies.

In an embodiment of the method according to the present invention, the method further comprises performing a selection to remove any remnant B-cells from the expanded population of PBLs. In another embodiment, the selection is performed by using beads selective for CD19 to remove the remnant B-cells. In another embodiment, the selection is performed by admixing the beads selective for CD19 with the expanded population of PBLs to form complexes of beads and any remnant B-cells and removing the complexes from the admixture. In another embodiment, the selection is performed by admixing magnetic beads selective for CD19 with the expanded population of PBLs to form complexes of magnetic beads and any remnant B-cells and using a magnet to remove the complexes from the admixture. In an embodiment of the invention, the beads selective for CD19 are beads conjugated to anti-CD19 antibody.

In an embodiment according to the present invention, the first cell culture medium contains about 3000 IU/mL of IL-2. In another embodiment, the second cell culture medium contains about 3000 IU/mL of IL-2. In yet another embodiment, the cultures in the culturing steps are incubated at 37° C. and under an atmosphere containing 5% CO₂.

In an embodiment of the invention, the method according to the present invention is performed over a period of about 9 to about 11 days. In another embodiment, the method is performed over a period of about 9 days. In another embodiment, the method is performed over a period of about 11 days.

In an embodiment of the invention, the patient is pretreated with an ITK inhibitor. In one embodiment, the ITK inhibitor is ibrutinib. In another embodiment, the patient has a liquid tumor. In another embodiment, the patient has a liquid tumor and is pretreated with an ITK inhibitor. In another embodiment, the patient has a liquid tumor, is refractory to treatment with an ITK inhibitor, and is pretreated with the ITK inhibitor.

In an embodiment of the invention, the patient suffers from leukemia. In another embodiment, the leukemia is chronic lymphocytic leukemia.

### BRIEF DESCRIPTION OF THE DRAWINGS

The foregoing summary, as well as the following detailed description of the invention, will be better understood when read in conjunction with the appended drawings.

FIG. 1 illustrates an exemplary embodiment of a PBL manufacturing process with B-cell depletion on Day 9.

FIG. 2 illustrates the design of experiments to compare the T-cell positive selection method using CTS Dynabeads CD3/28 to the T-cell negative selection method using either research grade Pan-T kit or a sequential anti-CD14, anti CD19 depletion method using CliniMACS microbeads.

FIG. 3 illustrates total PBL yields (in billions) for two samples extrapolated for an exemplary 9 day manufacturing process.

FIG. 4 illustrates total PBL yields (in billions) for two samples extrapolated for an exemplary 11 day manufacturing process.

FIG. 5 illustrates total viable cell counts (TVC) of PBLs from 50 mL of whole blood from two different patients on day 9 of an exemplary manufacturing process.

FIG. 6 illustrates fold expansion of PBLs from 50 mL of whole blood from the same patients as in FIG. 5 on day 9 of an exemplary manufacturing process.

FIG. 7 illustrates interferon-gamma levels (in pg/mL/5e5 cells) from the same patients as in FIG. 5 from an exemplary manufacturing process.

FIG. 8 illustrates interferon-gamma levels (in pg/mL) from the same patients as in FIG. 5 from an exemplary manufacturing process.

FIG. 9 illustrates an embodiment of projected doses of a PBL product.

FIG. 10A illustrates an exemplary embodiment of a PBL manufacturing process.

FIG. 10B illustrates an exemplary embodiment of a PBL manufacturing process with B-cell depletion.

FIG. 10C illustrates an exemplary embodiment of a PBL manufacturing process with B-cell depletion on Day 0.

FIG. 11 illustrates an exemplary embodiment of a PBL manufacturing process. Cryopreserved PBMCs obtained from peripheral blood of CLL patients were enriched for T-cells. Enriched fractions were expanded for a duration of 9-14 days in the presence of CTS™Dynabeads™ (αCD3/αCD28) and IL-2 to obtain PBLs.

FIG. 12 illustrates fold expansion of PBLs using a 9 day and 14 day expansion processes in treatment-naïve, pre-ibrutinib, and post-ibrutinib treated patients. Statistical significance is shown as: *p<0.05; **p≤0.01; and ***p≤0.001.

FIG. 13 illustrates interferon-gamma secretion by different groups of PBLs in response to non-specific TCR engagement. IFNγ secretion was assessed using an ELIspot assay. Data shown is IFNγ secreting T-cells per million PBLs. Statistical significance is shown as: *p<0.05; **p≤0.01; and ***p≤0.001.

FIGS. 14A-14H illustrate cytotoxicity of different groups of PBLs against autologous CD19+ cells. Cytotoxicity was assessed using a flow cytometry based cell-killing assay. Data samples are paired and depicted for four patients; FIGS. 14A, 14C, 14E, and 14G are pre-ibrutinib samples and FIGS. 14B, 14D, 14F, and 14H are post-ibrutinib samples.

FIG. 15 illustrates CD19+ target specificity as determined by HLA blockade experiments. HLA class I and class II molecules on cells were blocked using HLA blocking antibody cocktails.

FIGS. 16A-16E illustrate box plots representing gene expression levels related to different T-cell pathways as measured by nCounter CAR-T characterization panel. Gene expression is represented by the y-axis score. Scores were measured for melanoma TIL (labeled as “Final”), 14-day expanded PBLs from ibrutinib-treated patients (labeled as D.14), and 9-day expanded PBLs from ibrutinib-treated patients (labeled as D.9).

FIG. 17 illustrates fold expansion in a 9 day expansion process with B-cell depletion at Day 0. Circles represent IRuns 1, 2, 4, and 5, and the square represents MRun 5. As shown, B-cell depletion at Day 0, particularly in samples with a high initial B-cell content, does not appear to negatively affect fold expansion of T-cells over the 9 day process, even though the initial T-cell content may be partially depleted in the Day 0 B-cell depletion process (see FIG. 18).

FIGS. 18A and 18B illustrate Day 9 T cell yields versus total initial T-cells (FIG. 18A) and initial B-cell content (FIG. 18B) with B-cell depletion occurring either at Day 0 or at Day 9.

### BRIEF DESCRIPTION OF THE SEQUENCE LISTING

SEQ ID NO:1 is the amino acid sequence of the heavy chain of muromonab.

SEQ ID NO:2 is the amino acid sequence of the light chain of muromonab.

SEQ ID NO:3 is the amino acid sequence of a recombinant human IL-2 protein.

SEQ ID NO:4 is the amino acid sequence of aldesleukin.

SEQ ID NO:5 is the amino acid sequence of a recombinant human IL-4 protein.

SEQ ID NO:6 is the amino acid sequence of a recombinant human IL-7 protein.

SEQ ID NO:7 is the amino acid sequence of a recombinant human IL-15 protein.

SEQ ID NO:8 is the amino acid sequence of a recombinant human IL-21 protein.

### REFERENCE TO AN ELECTRONIC SEQUENCE LISTING

The contents of the electronic sequence listing (116983-5051-US01_sequence_listing.xml; Size: 12,547 bytes; and Date of Creation: Jul. 29, 2022) submitted with this application is herein incorporated by reference in its entirety.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

### Definitions

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “in vivo” refers to an event that takes place in a mammalian subject's body.

The term “ex vivo” refers to an event that takes place outside of a mammalian subject's body, in an artificial environment.

The term “in vitro” refers to an event that takes places in a test system. In vitro assays encompass cell-based assays in which alive or dead cells may be are employed and may also encompass a cell-free assay in which no intact cells are employed.

The term “rapid expansion” means an increase in the number of antigen-specific TILs of at least about 3-fold (or 4-, 5-, 6-, 7-, 8-, or 9-fold) over a period of a week, more preferably at least about 10-fold (or 20-, 30-, 40-, 50-, 60-, 70-, 80-, or 90-fold) over a period of a week, or most preferably at least about 100-fold over a period of a week. A number of rapid expansion protocols are described herein.

The terms “fragmenting,” “fragment,” and “fragmented,” as used herein to describe processes for disrupting a tumor, includes mechanical fragmentation methods such as crushing, slicing, dividing, and morcellating tumor tissue as well as any other method for disrupting the physical structure of tumor tissue.

The terms “peripheral blood mononuclear cells” and “PBMCs” refers to a peripheral blood cell having a round nucleus, including lymphocytes (T cells, B cells, NK cells) and monocytes. Optionally, the peripheral blood mononuclear cells are irradiated allogeneic peripheral blood mononuclear cells. PBMCs include antigen presenting cells. The term “PBLs” refers to peripheral blood lymphocytes and are T-cells expanded from peripheral blood. The terms PBL and TIL are used interchangeably herein.

The term “anti-CD3 antibody” refers to an antibody or variant thereof, e.g., a monoclonal antibody and including human, humanized, chimeric or murine antibodies which are directed against the CD3 receptor in the T cell antigen receptor of mature T cells. Anti-CD3 antibodies include OKT-3, also known as muromonab, and UCHT-1. Other anti-CD3 antibodies include, for example, otelixizumab, teplizumab, and visilizumab.

The term “OKT-3” (also referred to herein as “OKT3”) refers to a monoclonal antibody or biosimilar or variant thereof, including human, humanized, chimeric, or murine antibodies, directed against the CD3 receptor in the T cell antigen receptor of mature T cells, and includes commercially-available forms such as OKT-3 (30 ng/mL, MACS GMP CD3 pure, Miltenyi Biotech, Inc., San Diego, Calif., USA) and muromonab or variants, conservative amino acid substitutions, glycoforms, or biosimilars thereof. The amino acid sequences of the heavy and light chains of muromonab are given in Table 1 (SEQ ID NO:1 and SEQ ID NO:2). A hybridoma capable of producing OKT-3 is deposited with the American Type Culture Collection and assigned the ATCC accession number CRL 8001. A hybridoma capable of producing OKT-3 is also deposited with European Collection of Authenticated Cell Cultures (ECACC) and assigned Catalogue No. 86022706.

TABLE 1
Amino acid sequences of muromonab.

| Identifier | Sequence (One-Letter Amino Acid Symbols) |
|---|---|
| SEQ ID NO: 1 | QVQLQQSGAE LARPGASVKM SCKASGYTFT RYTMHWVKQR PGQGLEWIGY INPSRGYTNY 60 |
| Muromonab heavy | NQKFKDKATL TTDKSSSTAY MQLSSLTSED SAVYYCARYY DDHYCLDYWG QGTTLTVSSA 120 |
| chain | KTTAPSVYPL APVCGGTTGS SVTLGCLVKG yfpepvtltw NSGSLSSGVH TFPAVLQSDL 180 |
| | YTLSSSVTVT SSTWPSQSIT CNVAHPASST KVDKKIEPRP KSCDKTHTCP PCPAPELLGG 240 |
| | PSVFLFPPKP KDTLMISRTP EVTCVVVDVS HEDPEVKFNW YVDGVEVHNA KTKPREEQYN 300 |
| | STYRVVSVLT VLHQDWLNGK EYKCKVSNKA LPAPIEKTIS KAKGQPREPQ VYTLPPSRDE 360 |
| | LTKNQVSLTC LVKGFYPSDI AVEWESNGQP ENNYKTTPPV LDSDGSFFLY SKLTVDKSRW 420 |
| | QQGNVFSCSV MHEALHNHYT QKSLSLSPGK 450 |
| SEQ ID NO: 2 | QIVLTQSPAI MSASPGEKVT MTCSASSSVS YMNWYQQKSG TSPKRWIYDT SKLASGVPAH 60 |
| Muromonab light | FRGSGSGTSY SLTISGMEAE DAATYYCQOW SSNPFTFGSG TKLEINRADT APTVSIFPPS 120 |
| chain | SEQLTSGGAS VVCFLNNFYP KDINVKWKID GSERQNGVLN SWTDQDSKDS TYSMSSTLTL 180 |
| | TKDEYERHNS YTCEATHKTS TSPIVKSFNR NEC 213 |

The term “IL-2” (also referred to herein as “IL2”) refers to the T cell growth factor known as interleukin-2, and includes all forms of IL-2 including human and mammalian forms, conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated by reference herein. The amino acid sequence of recombinant human IL-2 suitable for use in the invention is given in Table 2 (SEQ ID NO:3). For example, the term IL-2 encompasses human, recombinant forms of IL-2 such as aldesleukin (PROLEUKIN, available commercially from multiple suppliers in 22 million IU per single use vials), as well as the form of recombinant IL-2 commercially supplied by CellGenix, Inc., Portsmouth, N.H., USA (CELLGRO GMP) or ProSpec-Tany TechnoGene Ltd., East Brunswick, N.J., USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The amino acid sequence of aldesleukin suitable for use in the invention is given in Table 2 (SEQ ID NO:4). The term IL-2 also encompasses pegylated forms of IL-2, as described herein, including the pegylated IL-2 prodrug NKTR-214, available from Nektar Therapeutics, South San Francisco, Calif., USA. NKTR-214 and pegylated IL-2 suitable for use in the invention is described in U.S. Patent Application Publication No. US 2014/0328791 A1 and International Patent Application Publication No. WO 2012/065086 A1, the disclosures of which are incorporated by reference herein. Alternative forms of conjugated IL-2 suitable for use in the invention are described in U.S. Pat. Nos. 4,766,106, 5,206,344, 5,089,261 and 4,902,502, the disclosures of which are incorporated by reference herein. Formulations of IL-2 suitable for use in the invention are described in U.S. Pat. No. 6,706,289, the disclosure of which is incorporated by reference herein.

TABLE 2
Amino acid sequences of interleukins.

| Identifier | Sequence (One-Letter Amino Acid Symbols) |
|---|---|
| SEQ ID NO: 3 | MAPTSSSTKK TQLQLEHLLL DLQMILNGIN NYKNPKLTRM LTFKFYMPKK ATELKHLQCL 60 |
| recombinant | EEELKPLEEV LNLAQSKNFH LRPRDLISNI NVIVLELKGS ETTFMCEYAD ETATIVEFLN 120 |
| human IL-2 | RWITFCQSII STLT 134 |
| (rhIL-2) | |
| SEQ ID NO: 4 | PTSSSTKKTQ LQLEHLLLDL QMILNGINNY KNPKLTRMLT FKFYMPKKAT ELKHLQCLEE 60 |
| Aldesleukin | ELKPLEEVLN LAQSKNFHLR PRDLISNINV IVLELKGSET TFMCEYADET ATIVEFLNRW 120 |
| | ITFSQSIIST LT 132 |
| SEQ ID NO: 5 | MHKCDITLQE IIKTLNSLTE QKTLCTELTV TDIFAASKNT TEKETFCRAA TVLRQFYSHH 60 |
| recombinant | EKDTRCLGAT AQQFHRHKQL IRFLKRLDRN LWGLAGLNSC PVKEANQSTL ENFLERLKTI 120 |
| human IL-4 | MREKYSKCSS 130 |
| (rhIL-4) | |
| SEQ ID NO: 6 | MDCDIEGKDG KQYESVLMVS IDQLLDSMKE IGSNCLNNEF NFFKRHICDA NKEGMFLFRA 60 |
| recombinant | ARKLRQFLKM NSTGDFDLHL LKVSEGTTIL LNCTGQVKGR KPAALGEAQP TKSLEENKSL 120 |
| human IL-7 | KEQKKLNDLC FLKRLLQEIK TCWNKILMGT KEH 153 |
| (rhIL-7) | |
| SEQ ID NO: 7 | MNWVNVISDL KKIEDLIQSM HIDATLYTES DVHPSCKVTA MKCFLLELQV ISLESGDASI 60 |
| recombinant | HDTVENLIIL ANNSLSSNGN VTESGCKECE ELEEKNIKEF LQSFVHIVQM PINTS 115 |
| human IL-15 | |
| (rhIL-15) | |
| SEQ ID NO: 8 | MQDRHMIRMR QLIDIVDQLK NYVNDLVPEF LPAPEDVETN CEWSAFSCFQ KAQLKSANTG 60 |
| recombinant | NNERIINVSI KKLKRKPPST NAGRRQKHRL TCPSCDSYEK KPPKEFLERF KSLLQKMIHQ 120 |
| human IL-21 | HLSSRTHGSE DS 132 |
| (rhIL-21) | |

The term “IL-4” (also referred to herein as “IL4”) refers to the cytokine known as interleukin 4, which is produced by Th2 T cells and by eosinophils, basophils, and mast cells. IL-4 regulates the differentiation of naïve helper T cells (Th0 cells) to Th2 T cells. Steinke and Borish, Respir. Res. 2001, 2, 66-70. Upon activation by IL-4, Th2 T cells subsequently produce additional IL-4 in a positive feedback loop. IL-4 also stimulates B cell proliferation and class II MHC expression, and induces class switching to IgE and IgG₁ expression from B cells. Recombinant human IL-4 suitable for use in the invention is commercially available from multiple suppliers, including ProSpec-Tany TechnoGene Ltd., East Brunswick, N.J., USA (Cat. No. CYT-211) and ThermoFisher Scientific, Inc., Waltham, Mass., USA (human IL-15 recombinant protein, Cat. No. Gibco CTP0043). The amino acid sequence of recombinant human IL-4 suitable for use in the invention is given in Table 2 (SEQ ID NO:5).

The term “IL-7” (also referred to herein as “IL7”) refers to a glycosylated tissue-derived cytokine known as interleukin 7, which may be obtained from stromal and epithelial cells, as well as from dendritic cells. Fry and Mackall, Blood 2002, 99, 3892-904. IL-7 can stimulate the development of T cells. IL-7 binds to the IL-7 receptor, a heterodimer consisting of IL-7 receptor alpha and common gamma chain receptor, which in a series of signals important for T cell development within the thymus and survival within the periphery. Recombinant human IL-7 suitable for use in the invention is commercially available from multiple suppliers, including ProSpec-Tany TechnoGene Ltd., East Brunswick, N.J., USA (Cat. No. CYT-254) and ThermoFisher Scientific, Inc., Waltham, Mass., USA (human IL-7 recombinant protein, Cat. No. Gibco PHC0071). The amino acid sequence of recombinant human IL-7 suitable for use in the invention is given in Table 2 (SEQ ID NO:6).

The term “IL-15” (also referred to herein as “IL15”) refers to the T cell growth factor known as interleukin-15, and includes all forms of IL-15 including human and mammalian forms, conservative amino acid substitutions, glycoforms, biosimilars, and variants thereof. IL-15 is described, e.g., in Fehniger and Caligiuri, Blood 2001, 97, 14-32, the disclosure of which is incorporated by reference herein. IL-15 shares β and γ signaling receptor subunits with IL-2. Recombinant human IL-15 is a single, non-glycosylated polypeptide chain containing 114 amino acids (and an N-terminal methionine) with a molecular mass of 12.8 kDa. Recombinant human IL-15 is commercially available from multiple suppliers, including ProSpec-Tany TechnoGene Ltd., East Brunswick, N.J., USA (Cat. No. CYT-230-b) and ThermoFisher Scientific, Inc., Waltham, Mass., USA (human IL-15 recombinant protein, Cat. No. 34-8159-82). The amino acid sequence of recombinant human IL-15 suitable for use in the invention is given in Table 2 (SEQ ID NO:7).

The term “IL-21” (also referred to herein as “IL21”) refers to the pleiotropic cytokine protein known as interleukin-21, and includes all forms of IL-21 including human and mammalian forms, conservative amino acid substitutions, glycoforms, biosimilars, and variants thereof. IL-21 is described, e.g., in Spolski and Leonard, Nat. Rev. Drug. Disc. 2014, 13, 379-95, the disclosure of which is incorporated by reference herein. IL-21 is primarily produced by natural killer T cells and activated human CD4⁺ T cells. Recombinant human IL-21 is a single, non-glycosylated polypeptide chain containing 132 amino acids with a molecular mass of 15.4 kDa. Recombinant human IL-21 is commercially available from multiple suppliers, including ProSpec-Tany TechnoGene Ltd., East Brunswick, N.J., USA (Cat. No. CYT-408-b) and ThermoFisher Scientific, Inc., Waltham, Mass., USA (human IL-21 recombinant protein, Cat. No. 14-8219-80). The amino acid sequence of recombinant human IL-21 suitable for use in the invention is given in Table 2 (SEQ ID NO:8).

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

The terms “antibody” and its plural form “antibodies” refer to whole immunoglobulins and any antigen-binding fragment (“antigen-binding portion”) or single chains thereof. An “antibody” further refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions of an antibody may be further subdivided into regions of hypervariability, which are referred to as complementarity determining regions (CDR) or hypervariable regions (HVR), and which can be interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen epitope or epitopes. The constant regions of the antibodies may mediate the binding of the immunoglobulin to host tissues or factors, including various cells of the immune system (e.g., effector cells) and the first component (C1q) of the classical complement system.

The term “antigen” refers to a substance that induces an immune response. In some embodiments, an antigen is a molecule capable of being bound by an antibody or a TCR if presented by major histocompatibility complex (MHC) molecules. The term “antigen”, as used herein, also encompasses T cell epitopes. An antigen is additionally capable of being recognized by the immune system. In some embodiments, an antigen is capable of inducing a humoral immune response or a cellular immune response leading to the activation of B lymphocytes and/or T lymphocytes. In some cases, this may require that the antigen contains or is linked to a Th cell epitope. An antigen can also have one or more epitopes (e.g., B- and T-epitopes). In some embodiments, an antigen will preferably react, typically in a highly specific and selective manner, with its corresponding antibody or TCR and not with the multitude of other antibodies or TCRs which may be induced by other antigens.

The terms “monoclonal antibody,” “mAb,” “monoclonal antibody composition,” or their plural forms refer to a preparation of antibody molecules of single molecular composition. A monoclonal antibody composition displays a single binding specificity and affinity for a particular epitope. Monoclonal antibodies specific to certain receptors can be made using knowledge and skill in the art of injecting test subjects with suitable antigen and then isolating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

The terms “antigen-binding portion” or “antigen-binding fragment” of an antibody (or simply “antibody portion” or “fragment”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a domain antibody (dAb) fragment (Ward, et al., Nature, 1989, 341, 544-546), which may consist of a V_{H} or a V_{L} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see, e.g., Bird, et al., Science 1988, 242, 423-426; and Huston, et al., Proc. Natl. Acad. Sci. USA 1988, 85, 5879-5883). Such scFv antibodies are also intended to be encompassed within the terms “antigen-binding portion” or “antigen-binding fragment” of an antibody. These antibody fragments are obtained using conventional techniques known to those with skill in the art, and the fragments are screened for utility in the same manner as are intact antibodies.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). The term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In an embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (such as a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, including a conjugate of the antibody and another active pharmaceutical ingredient or antibody. The terms “conjugate,” “antibody-drug conjugate”, “ADC,” or “immunoconjugate” refers to an antibody, or a fragment thereof, conjugated to another therapeutic moiety, which can be conjugated to antibodies described herein using methods available in the art.

The terms “humanized antibody,” “humanized antibodies,” and “humanized” are intended to refer to antibodies in which CDR sequences derived from the germline of another mammalian species, such as a mouse, have been grafted onto human framework sequences. Additional framework region modifications may be made within the human framework sequences. Humanized forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a 15 hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones, et al., Nature 1986, 321, 522-525; Riechmann, et al., Nature 1988, 332, 323-329; and Presta, Curr. Op. Struct. Biol. 1992, 2, 593-596. The antibodies described herein may also be modified to employ any Fc variant which is known to impart an improvement (e.g., reduction) in effector function and/or FcR binding. The Fc variants may include, for example, any one of the amino acid substitutions disclosed in International Patent Application Publication Nos. WO 1988/07089 A1, WO 1996/14339 A1, WO 1998/05787 A1, WO 1998/23289 A1, WO 1999/51642 A1, WO 99/58572 A1, WO 2000/09560 A2, WO 2000/32767 A1, WO 2000/42072 A2, WO 2002/44215 A2, WO 2002/060919 A2, WO 2003/074569 A2, WO 2004/016750 A2, WO 2004/029207 A2, WO 2004/035752 A2, WO 2004/063351 A2, WO 2004/074455 A2, WO 2004/099249 A2, WO 2005/040217 A2, WO 2005/070963 A1, WO 2005/077981 A2, WO 2005/092925 A2, WO 2005/123780 A2, WO 2006/019447 A1, WO 2006/047350 A2, and WO 2006/085967 A2; and U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784; the disclosures of which are incorporated by reference herein.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

A “diabody” is a small antibody fragment with two antigen-binding sites. The fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., European Patent No. EP 404,097, International Patent Publication No. WO 93/11161; and Bolliger, et al., Proc. Natl. Acad. Sci. USA 1993, 90, 6444-6448.

The term “glycosylation” refers to a modified derivative of an antibody. An aglycoslated antibody lacks glycosylation. Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Aglycosylation may increase the affinity of the antibody for antigen, as described in U.S. Pat. Nos. 5,714,350 and 6,350,861. Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see e.g. U.S. Patent Publication No. 2004/0110704 or Yamane-Ohnuki, et al., Biotechnol. Bioeng., 2004, 87, 614-622). As another example, European Patent No. EP 1,176,195 describes a cell line with a functionally disrupted FUT8 gene, which encodes a fucosyl transferase, such that antibodies expressed in such a cell line exhibit hypofucosylation by reducing or eliminating the alpha 1,6 bond-related enzyme, and also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). International Patent Publication WO 03/035835 describes a variant CHO cell line, Lec 13 cells, with reduced ability to attach fucose to Asn(297)-linked carbohydrates, also resulting in hypofucosylation of antibodies expressed in that host cell (see also Shields, et al., J. Biol. Chem. 2002, 277, 26733-26740. International Patent Publication WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana, et al., Nat. Biotech. 1999, 17, 176-180). Alternatively, the fucose residues of the antibody may be cleaved off using a fucosidase enzyme. For example, the fucosidase alpha-L-fucosidase removes fucosyl residues from antibodies as described in Tarentino, et al., Biochem. 1975, 14, 5516-5523.

“Pegylation” refers to a modified antibody, or a fragment thereof, that typically is reacted with polyethylene glycol (PEG), such as a reactive ester or aldehyde derivative of PEG, under conditions in which one or more PEG groups become attached to the antibody or antibody fragment. Pegylation may, for example, increase the biological (e.g., serum) half life of the antibody. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀)alkoxy- or aryloxy-polyethylene glycol or polyethylene glycol-maleimide. The antibody to be pegylated may be an aglycosylated antibody. Methods for pegylation are known in the art and can be applied to the antibodies of the invention, as described for example in European Patent Nos. EP 0154316 and EP 0401384 and U.S. Pat. No. 5,824,778, the disclosures of each of which are incorporated by reference herein.

The terms “fusion protein” or “fusion polypeptide” refer to proteins that combine the properties of two or more individual proteins. Such proteins have at least two heterologous polypeptides covalently linked either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order and may include more than one of either or both of the constituent polypeptides. The term encompasses conservatively modified variants, polymorphic variants, alleles, mutants, subsequences, interspecies homologs, and immunogenic fragments of the antigens that make up the fusion protein. Fusion proteins of the disclosure can also comprise additional copies of a component antigen or immunogenic fragment thereof. The fusion protein may contain one or more binding domains linked together and further linked to an Fc domain, such as an IgG Fc domain. Fusion proteins may be further linked together to mimic a monoclonal antibody and provide six or more binding domains. Fusion proteins may be produced by recombinant methods as is known in the art. Preparation of fusion proteins are known in the art and are described, e.g., in International Patent Application Publication Nos. WO 1995/027735 A1, WO 2005/103077 A1, WO 2008/025516 A1, WO 2009/007120 A1, WO 2010/003766 A1, WO 2010/010051 A1, WO 2010/078966 A1, U.S. Patent Application Publication Nos. US 2015/0125419 A1 and US 2016/0272695 A1, and U.S. Pat. No. 8,921,519, the disclosures of each of which are incorporated by reference herein.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, or coding regions from different sources. Similarly, a heterologous protein indicates that the protein comprises two or more subsequences that are not found in the same relationship to each other in nature (e.g., a fusion protein).

The term “conservative amino acid substitutions” in means amino acid sequence modifications which do not abrogate the binding of an antibody or fusion protein to the antigen. Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in an antibody is preferably replaced with another amino acid residue from the same class. Methods of identifying amino acid conservative substitutions which do not eliminate antigen binding are well-known in the art (see, e.g., Brummell, et al., Biochemistry 1993, 32, 1180-1187; Kobayashi, et al., Protein Eng. 1999, 12, 879-884 (1999); and Burks, et al., Proc. Natl. Acad. Sci. USA 1997, 94, 412-417.

The terms “sequence identity,” “percent identity,” and “sequence percent identity” (or synonyms thereof, e.g., “99% identical”) in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

As used herein, the term “variant” encompasses but is not limited to antibodies or fusion proteins which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody. The term variant also includes pegylated antibodies or proteins.

Nucleic acid sequences implicitly encompass conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. Batzer, et al., Nucleic Acid Res. 1991, 19, 5081; Ohtsuka, et al., J. Biol. Chem. 1985, 260, 2605-2608; Rossolini, et al., Mol. Cell. Probes 1994, 8, 91-98. The term nucleic acid is used interchangeably with cDNA, mRNA, oligonucleotide, and polynucleotide.

The term “biosimilar” means a biological product, including a monoclonal antibody or protein, that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. They can consist of relatively small molecules such as human insulin or erythropoietin, or complex molecules such as monoclonal antibodies. For example, if the reference IL-2 protein is aldesleukin (PROLEUKIN), a protein approved by drug regulatory authorities with reference to aldesleukin is a “biosimilar to” aldesleukin or is a “biosimilar thereof” of aldesleukin. In Europe, a similar biological or “biosimilar” medicine is a biological medicine that is similar to another biological medicine that has already been authorized for use by the European Medicines Agency (EMA). The relevant legal basis for similar biological applications in Europe is Article 6 of Regulation (EC) No 726/2004 and Article 10(4) of Directive 2001/83/EC, as amended and therefore in Europe, the biosimilar may be authorized, approved for authorization or subject of an application for authorization under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA and published on its website. A biosimilar as described herein may be similar to the reference medicinal product by way of quality characteristics, biological activity, mechanism of action, safety profiles and/or efficacy. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorized by the EMA. However, in some instances, the biosimilar may be compared to a biological medicinal product which has been authorized outside the European Economic Area (a non-EEA authorized “comparator”) in certain studies. Such studies include for example certain clinical and in vivo non-clinical studies. As used herein, the term “biosimilar” also relates to a biological medicinal product which has been or may be compared to a non-EEA authorized comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97% or greater to the amino acid sequence of its reference medicinal product, e.g., 97%, 98%, 99% or 100%. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorized or considered suitable for authorization. In certain circumstances, the biosimilar exhibits different binding characteristics as compared to the reference medicinal product, wherein the different binding characteristics are considered by a Regulatory Authority such as the EMA not to be a barrier for authorization as a similar biological product. The term “biosimilar” is also used synonymously by other national and regional regulatory agencies.

The term “hematological malignancy” refers to mammalian cancers and tumors of the hematopoietic and lymphoid tissues, including but not limited to tissues of the blood, bone marrow, lymph nodes, and lymphatic system. Hematological malignancies may result in the formation of a “liquid tumor.” Hematological malignancies include, but are not limited to, acute lymphoblastic leukemia (ALL), chronic lymphocytic lymphoma (CLL), small lymphocytic lymphoma (SLL), acute myeloid leukemia (AML), chronic myelogenous leukemia (CML), acute monocytic leukemia (AMoL), Hodgkin's lymphoma, and non-Hodgkin's lymphomas. The term “B cell hematological malignancy” refers to hematological malignancies that affect B cells.

The term “liquid tumor” refers to an abnormal mass of cells that is fluid in nature. Liquid tumor cancers include, but are not limited to, leukemias, myelomas, and lymphomas, as well as other hematological malignancies. TILs obtained from liquid tumors, including liquid tumors resident in bone marrow, may also be referred to herein as marrow infiltrating lymphocytes (MILs). TILs obtained from liquid tumors, including liquid tumors circulating in peripheral blood, may also be referred to herein as PBLs. The terms MIL, TIL, and PBL are used interchangeably herein and differ only based on the tissue type from which the cells are derived.

The term “biopsy” refers to any medical procedure used to obtain cancerous cells, including bone marrow biopsy.

The terms “acute myeloid leukemia” or “AML” refers to cancers of the myeloid blood cell lines, which are also known in the art as acute myelogenous leukemia and acute nonlymphocytic leukemia. Although AML is a liquid tumor, some manifestations of AML, including extramedullary manifestations such as chloroma, exhibit properties of a solid tumor, but are classified herein as a liquid tumor.

The term “microenvironment,” as used herein, may refer to the solid or hematological tumor microenvironment as a whole or to an individual subset of cells within the microenvironment. The tumor microenvironment, as used herein, refers to a complex mixture of “cells, soluble factors, signaling molecules, extracellular matrices, and mechanical cues that promote neoplastic transformation, support tumor growth and invasion, protect the tumor from host immunity, foster therapeutic resistance, and provide niches for dominant metastases to thrive,” as described in Swartz, et al., Cancer Res., 2012, 72, 2473. Although tumors express antigens that should be recognized by T cells, tumor clearance by the immune system is rare because of immune suppression by the microenvironment.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

By “tumor infiltrating lymphocytes” or “TILs” herein is meant a population of cells originally obtained as white blood cells that have left the bloodstream of a subject and migrated into a tumor. TILs include, but are not limited to, CD8+ cytotoxic T cells (lymphocytes), Th1 and Th17 CD4+ T cells, natural killer cells, dendritic cells and M1 macrophages. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated as discussed herein, including, but not limited to bulk TILs, expanded TILs (“REP TILs”) as well as “reREP TILs” as discussed herein.

TILs can generally be defined either biochemically, using cell surface markers, or functionally, by their ability to infiltrate tumors and effect treatment. TILs can be generally categorized by expressing one or more of the following biomarkers: CD4, CD8, TCR αβ, CD27, CD28, CD56, CCR7, CD45Ra, CD95, PD-1, and CD25. Additionally and alternatively, TILs can be functionally defined by their ability to infiltrate solid tumors upon reintroduction into a patient. TILS may further be characterized by potency—for example, TILS may be considered potent if, for example, interferon (IFN) release is greater than about 50 pg/mL, greater than about 100 pg/mL, greater than about 150 pg/mL, or greater than about 200 pg/mL.

By “cryopreserved TILs” (or cryopreserved MILs or PBLs) herein is meant that TILs, either primary, bulk, or expanded (REP TILs), are treated and stored in the range of about −150° C. to −60° C. General methods for cryopreservation are also described elsewhere herein, including in the Examples. For clarity, “cryopreserved TILs” are distinguishable from frozen tissue samples which may be used as a source of primary TILs.

By “thawed cryopreserved TILs” (or thawed MILs or PBLs) herein is meant a population of TILs that was previously cryopreserved and then treated to return to room temperature or higher, including but not limited to cell culture temperatures or temperatures wherein TILs may be administered to a patient.

By “population of cells” (including TILs) herein is meant a number of cells that share common traits. In general, populations generally range from 1×10⁶ to 1×10¹⁰ in number, with different TIL populations comprising different numbers. For example, initial growth of primary TILs in the presence of IL-2 results in a population of bulk TILs of roughly 1×10⁸ cells. REP expansion is generally done to provide populations of 1.5×10⁹ to 1.5×10¹⁰ cells for infusion.

In general, TILs are initially obtained from a patient tumor sample (“primary TILs”) and then expanded into a larger population for further manipulation as described herein, optionally cyropreserved, restimulated as outlined herein and optionally evaluated for phenotype and metabolic parameters as an indication of TIL health.

In general, the harvested cell suspension is called a “primary cell population” or a “freshly harvested” cell population.

In general, as discussed herein, the TILs are initially prepared by obtaining a primary population of TILs from a tumor resected from a patient as discussed herein (the “primary cell population” or “first cell population”). This is followed with an initial bulk expansion utilizing a culturing of the cells with IL-2, forming a second population of cells (sometimes referred to herein as the “bulk TIL population” or “second population”).

The term “cytotoxic lymphocyte” includes cytotoxic T (CTL) cells (including CD8⁺ cytotoxic T lymphocytes and CD4⁺ T-helper lymphocytes), natural killer T (NKT) cells and natural killer (NK) cells. Cytotoxic lymphocytes can include, for example, peripheral blood-derived αβ TCR-positive or γδ TCR-positive T cells activated by tumor associated antigens and/or transduced with tumor specific chimeric antigen receptors or T-cell receptors, and tumor-infiltrating lymphocytes (TILs).

The term “central memory T cell” refers to a subset of T cells that in the human are CD45RO+ and constitutively express CCR7 (CCR7h i) and CD62L (CD62 hi). The surface phenotype of central memory T cells also includes TCR, CD3, CD127 (IL-7R), and IL-15R. Transcription factors for central memory T cells include BCL-6, BCL-6B, MBD2, and BMII. Central memory T cells primarily secret IL-2 and CD40L as effector molecules after TCR triggering. Central memory T cells are predominant in the CD4 compartment in blood, and in the human are proportionally enriched in lymph nodes and tonsils.

The term “effector memory T cell” refers to a subset of human or mammalian T cells that, like central memory T cells, are CD45R0+, but have lost the constitutive expression of CCR7 (CCR7lo) and are heterogeneous or low for CD62L expression (CD62Llo). The surface phenotype of central memory T cells also includes TCR, CD3, CD127 (IL-7R), and IL-15R. Transcription factors for central memory T cells include BLIMP1. Effector memory T cells rapidly secret high levels of inflammatory cytokines following antigenic stimulation, including interferon-γ, IL-4, and IL-5. Effector memory T cells are predominant in the CD8 compartment in blood, and in the human are proportionally enriched in the lung, liver, and gut. CD8+ effector memory T cells carry large amounts of perforin. The term “closed system” refers to a system that is closed to the outside environment. Any closed system appropriate for cell culture methods can be employed with the methods of the present invention. Closed systems include, for example, but are not limited to closed G-containers. Once a tumor segment is added to the closed system, the system is no opened to the outside environment until the TILs are ready to be administered to the patient.

In some embodiments, methods of the present disclosure further include a “pre-REP” stage in which tumor tissue or cells from tumor tissue are grown in standard lab media (including without limitation RPMI) and treated the with reagents such as irradiated feeder cells and anti-CD3 antibodies to achieve a desired effect, such as increase in the number of TILS and/or an enrichment of the population for cells containing desired cell surface markers or other structural, biochemical or functional features. The pre-REP stage may utilize lab grade reagents (under the assumption that the lab grade reagents get diluted out during a later REP stage), making it easier to incorporate alternative strategies for improving TIL production. Therefore, in some embodiments, the disclosed TLR agonist and/or peptide or peptidomimetics can be included in the culture medium during the pre-REP stage. The pre-REP culture can in some embodiments, include IL-2. The present invention is directed in preferred aspects to novel methods of augmenting REPs with one or more additional restimulation protocols, also referred to herein as a “restimulation Rapid Expansion Protocol” or “reREP”, which leads surprisingly to expanded memory T cell subsets, including the memory effector T cell subset, and/or to markes enhancement in the glycolytic respiration as compared to freshly harvested TILs or thawed cryopreserved TILs for the restimulated TILs (sometimes referred to herein as “reTILs”). That is, by using a reREP procedure on cyropreserved TILs, patients can receive highly metabolically active, healthy TILs, leading to more favorable outcomes.

When “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the genetically modified cytotoxic lymphocytes described herein may be administered at a dosage of 10⁴ to 10¹¹ cells/kg body weight (e.g., 10⁵ to 10⁶, 10⁵ to 10¹⁰, 10⁵ to 10¹¹, 10⁶ to 10¹⁰, 10⁶ to 10¹¹, 10⁷ to 10¹¹, 10⁷ to 10¹⁰, 10⁸ to 10¹¹, 10⁸ to 10¹⁰, 10⁹ to 10¹¹, or 10⁹ to 10¹⁰ cells/kg body weight), including all integer values within those ranges. Genetically modified cytotoxic lymphocytes compositions may also be administered multiple times at these dosages. The genetically modified cytotoxic lymphocytes can be administered by using infusion techniques that are commonly known in immunotherapy (see, e.g., Rosenberg et al., New Eng. J. of Med. 319: 1676, 1988). The optimal dosage and treatment regime for a particular patient can readily be determined by one skilled in the art of medicine by monitoring the patient for signs of disease and adjusting the treatment accordingly.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of.”

Methods of Expanding Peripheral Blood Lymphocytes (PBLs) from Peripheral Blood

In an embodiment of the invention, PBLs are expanded using the processes described herein. In an embodiment of the invention, the method comprises obtaining a PBMC sample from whole blood. In an embodiment, the method comprises enriching T-cells by isolating pure T-cells from PBMCs using positive selection of a CD3+/CD28+ fraction, as follows. Thaw the cryopreserved PBMCs in a 37° C. waterbath. Transfer the thawed PBMCs into a 50 mL conical tube and mix well. Divide the cell suspension into two equal portions into two appropriately labelled 15 mL polystyrene conical tubes. Pellet the cells in the 15 mL tubes via centrifugation 400 g for 5 minutes at 24° C. (acceleration=9, deceleration=9). During centrifugation, mix the CTS Dynabeads (CD3/CD28) by placing on a rocker for at least 5 minutes. Remove the cells from the centrifuge and aspirate all the media. Cap tubes and scrape them along a rough surface (such as a tube rack) to help break up cell pellet. Calculate and record the number of CD3+ viable cells in a tube labelled appropriately (for example, “Method #1: Number of CD3+ viable cells=% CD3+ cells*TVC” (total viable cells). Resuspend the cells in a tube labelled appropriately (for example, “Method 1”) so that the concentration of the viable T-cells is 1e7/mL using wash buffer (sterile phosphate buffered saline (PBS), 1% Human Serum Albumin, 10 U/mL Dnase). Add the washed CTS DynaBeads (CD3/28) at 3 beads: 1 T-cell ratio by transferring the volume as calculated above. Incubate the sample with the Dynabeads, in a microtube covered with foil, on a rocker (1-3 RPM end to end) at room temperature for 30 minutes in the dark. After 30 minutes of incubation, place the sample in a 15 mL conical tube, rinse the microtube with 1 mL of CM2+IL-2 (3000 IU/mL) and transfer to the 15 mL tube. Bring the volume up to 10 mL using CM2+IL-2 and mix well using a pipettor. Place the tube on the DynaMag-15 for one to two minutes for positive selection of the bead-bound CD3+ cells. Decant the cell suspension (negative portion) into a 50 mL conical tube labelled appropriately (for example, “Method #1—no T cell fraction”). Immediately add 10 mL of CM2 media with IL-2 (3000 IU/mL) to the 15 mL tube that contains the bead-bound cells and mix. Place the tube on the Dynamag-15 for one to two minutes. Decant the cell suspension (residual negative portion) into the 50 mL conical tube labeled appropriately (for example, “Method #1—no T cell fraction”). Immediately add 5 mL of CM2 media with IL-2 (3000 IU/mL) to the 15 mL tube that contains the bead-bound cells and mix. Relabel the tube appropriately (for example, “Method #1—T cell fraction”). Count negative and positive portions. Obtain about 5e5 cells from each of the negative and the positive portions for flow analysis (CD3/4/8/19/14) of the fresh sample. CD3+CD8+ cells are CTLs, CD3+CD4+ cells are helper T-cells, CD19 cells are B-cells, and CD14+ cells are macrophages. Cryopreserve the leftover negative portion. Proceed with the culture of the positive T-cell enriched portion along with the Dynabeads.

On Day 0, to each of two G-REXSM flasks, place 1e6 viable T-cells. Label the flasks appropriately (for example, “Method #1”). Alternatively, to each G-REX 10M, place a minimum of 2e6 viable T cells. Slowly bring up the volume of the media in each G-REXSM flask to 20 mL of CM2 supplemented with 3000 IU IL-2/mL or to 40 mL in each G-REX10M. Place the flasks in the incubator (37° C. 5% CO₂).

On Day 4, add media. If cultured in G-REX 5M, add 20 mL of CM4+IL-2 (3000 IU/mL). If cultured in G-REX 10M, add 40 mL of CM4+IL-2 (3000 IU/mL).

On Day 7, add media. If cultured in G-REX 5M, add 10 mL of CM4+IL-2 (3000 IU/mL). If cultured in G-REX 10M, add 20 mL of CM4+IL-2 (3000 IU/mL).

Cells may be harvested on Day 9 or Day 11.

On the day of harvest, harvest one G-REX flask from each enrichment condition. Reduce the volume in the media to about 10% without disturbing the cells. Save two 1 mL samples for metabolite analysis at −20° C. freezer. Resuspend the cells and harvest in a 50 mL conical labelled appropriately (for example, “Method #1”). Add about 10 mL of Plasmalyte+1% HSA to each 50 mL tube. Place the conical tube in a Dynamag-50 for one to two minutes for bead removal. Using a 5 or 10 mL pipette, remove the cell suspension into anther 50 mL conical tube labelled Method #1 final. Immediately add 10 mL of Plasmalyte+1% HSA into the tubes in the Dynamag-50. Remove them from the magnet and mix, then return to the magnet. Place the 50 mL conicals again on the DynaMag-50 for 2 minutes to rinse. Using a 5 or 10 mL pipette, remove the cell suspension into the 50 mL conical tube labelled appropriately (for example, “Method #1 final”). Remove a sample for cell count and viability and for bead residual count. Cryopreserve the final product in vials using chilled freeze media (for example, 49.9% Plasmalyte-A, 0.5% HSA and 50% CS10).

In an embodiment, the invention provides a method for expanding peripheral blood lymphocytes (PBLs) from peripheral blood comprising:
a. Obtaining a sample of peripheral blood mononuclear cells (PBMCs) from the peripheral blood of a patient, wherein said sample is optionally cryopreserved and the patient is optionally pretreated with an ITK inhibitor;
b. Optionally washing the PBMCs by centrifugation;
c. Admixing magnetic beads selective for CD3 and CD28 to the PBMCs to form an admixture of the beads and the PBMCs;
d. Seeding the admixture of the beads and the PBMCs into a gas-permeable container and co-culturing said PBMCs in media comprising about 3000 IU/mL of IL-2 in for about 4 to about 6 days;
e. Feeding said PBMCs using media comprising about 3000 IU/mL of IL-2, and co-culturing said PBMCs for about 5 days, such that the total co-culture period of steps d and e is about 9 to about 11 days;
f. Harvesting PBMCs from media;
g. Removing the magnetic beads selective for CD3 and CD28 from the harvested PBMCs by using a magnet;
h. Removing residual B-cells from the harvested PBMCs using magnetic-activated cell sorting and beads selective for CD19 to provide a PBL product;
i. Washing and concentrating the PBL product using a cell harvester; and
j. Formulating and optionally cryopreserving the PBL product,
   wherein the ITK inhibitor is optionally an ITK inhibitor that covalently binds to ITK.

In an embodiment, PBMCs are isolated from a whole blood sample. In an embodiment, the PBMC sample is used as the starting material to expand the PBLs. In an embodiment, the PBMC sample is cryopreserved prior to the expansion process. In another embodiment, a fresh PBMC sample is used as the starting material to expand the PBLs. In an embodiment of the invention, T-cells are isolated from PBMCs using methods known in the art. In an embodiment, the T-cells are isolated using a Human Pan T-cell isolation kit and LS columns. In an embodiment of the invention, T-cells are isolated from PBMCs using antibody selection methods known in the art, for example, CD19 negative selection.

In an embodiment of the invention, the process is performed over about 7 days, about 8 days, about 9 days, about 10 days, about 11 days, about 12 days, about 13 days, or about 14 days. In another embodiment, the process is performed over about 7 days. In another embodiment, the process is performed over about 14 days.

In an embodiment of the invention, the PBMCs are cultured with antiCD3/antiCD28 antibodies. In an embodiment, any available antiCD3/antiCD28 product is useful in the present invention. In an embodiment of the invention, the commercially available product used are DynaBeads®. In an embodiment, the DynaBeads® are cultured with the PBMCs in a ratio of 1:1 (beads:cells). In another embodiment, the antibodies are DynaBeads® cultured with the PBMCs in a ratio of 1.5:1, 2:1, 2.5:1, 3:1, 3.5:1, 4:1, 4.5:1, or 5:1 (beads:cells). In an embodiment of the invention, the antibody culturing steps and/or the step of restimulating cells with antibody is performed over a period of from about 2 to about 6 days, from about 3 to about 5 days, or for about 4 days. In an embodiment of the invention, the antibody culturing step is performed over a period of about 2 days, 3 days, 4 days, 5 days, or 6 days.

In an embodiment, the PBMC sample is cultured with IL-2. In an embodiment of the invention, the cell culture medium used for expansion of the PBLs from PBMCs comprises IL-2 at a concentration selected from the group consisting of about 100 IU/mL, about 200 IU/mL, about 300 IU/mL, about 400 IU/mL, about 100 IU/mL, about 100 IU/mL, about 100 IU/mL, about 100 IU/mL, about 100 IU/mL, about 500 IU/mL, about 600 IU/mL, about 700 IU/mL, about 800 IU/mL, about 900 IU/mL, about 1,000 IU/mL, about 1,100 IU/mL, about 1,200 IU/mL, about 1,300 IU/mL, about 1,400 IU/mL, about 1,500 IU/mL, about 1,600 IU/mL, about 1,700 IU/mL, about 1,800 IU/mL, about 1,900 IU/mL, about 2,000 IU/mL, about 2,100 IU/mL, about 2,200 IU/mL, about 2,300 IU/mL, about 2,400 IU/mL, about 2,500 IU/mL, about 2,600 IU/mL, about 2,700 IU/mL, about 2,800 IU/mL, about 2,900 IU/mL, about 3,000 IU/mL, about 3,100 IU/mL, about 3,200 IU/mL, about 3,300 IU/mL, about 3,400 IU/mL, about 3,500 IU/mL, about 3,600 IU/mL, about 3,700 IU/mL, about 3,800 IU/mL, about 3,900 IU/mL, about 4,000 IU/mL, about 4,100 IU/mL, about 4,200 IU/mL, about 4,300 IU/mL, about 4,400 IU/mL, about 4,500 IU/mL, about 4,600 IU/mL, about 4,700 IU/mL, about 4,800 IU/mL, about 4,900 IU/mL, about 5,000 IU/mL, about 5,100 IU/mL, about 5,200 IU/mL, about 5,300 IU/mL, about 5,400 IU/mL, about 5,500 IU/mL, about 5,600 IU/mL, about 5,700 IU/mL, about 5,800 IU/mL, about 5,900 IU/mL, about 6,000 IU/mL, about 6,500 IU/mL, about 7,000 IU/mL, about 7,500 IU/mL, about 8,000 IU/mL, about 8,500 IU/mL, about 9,000 IU/mL, about 9,500 IU/mL, and about 10,000 IU/mL.

In an embodiment of the invention, the starting cell number of PBMCs for the expansion process is from about 25,000 to about 1,000,000, from about 30,000 to about 900,000, from about 35,000 to about 850,000, from about 40,000 to about 800,000, from about 45,000 to about 800,000, from about 50,000 to about 750,000, from about 55,000 to about 700,000, from about 60,000 to about 650,000, from about 65,000 to about 600,000, from about 70,000 to about 550,000, preferably from about 75,000 to about 500,000, from about 80,000 to about 450,000, from about 85,000 to about 400,000, from about 90,000 to about 350,000, from about 95,000 to about 300,000, from about 100,000 to about 250,000, from about 105,000 to about 200,000, or from about 110,000 to about 150,000. In an embodiment of the invention, the starting cell number of PBMCs is about 138,000, 140,000, 145,000, or more. In another embodiment, the starting cell number of PBMCs is about 28,000. In another embodiment, the starting cell number of PBMCs is about 62,000. In another embodiment, the starting cell number of PBMCs is about 338,000. In another embodiment, the starting cell number of PBMCs is about 336,000. In another embodiment, the starting cell number of PBMCs is 1 million, 2 million, 3 million, 4 million, 5 million, 6 million, 7 million, 8 million, 9 million, 10 million or more. In another embodiment, the starting cell number of PBMCs is 1 million to 10 million, 2 million to 9 million, 3 million to 8 million, 4 million to 7 million, or 5 million to 6 million. In another embodiment, the starting cell number of PBMCs is about 4 million. In yet another embodiment, the starting cell number of PBMCs is at least about 4 million, at least about 5 million, or at least about 6 million or more.

In an embodiment of the invention, the cells are grown in a GRex 24 well plate. In an embodiment of the invention, a comparable well plate is used. In an embodiment, the starting material for the expansion is about 5×10⁵ T-cells per well. In an embodiment of the invention, there are 1×10⁶ cells per well. In an embodiment of the invention, the number of cells per well is sufficient to seed the well and expand the T-cells.

In an embodiment of the invention, the cells are grown in a GRex100MCS container. In an embodiment of the invention, a comparable container is used. In an embodiment, the starting material for expansion is seeded at a density of about 25,000 to about 50,000 T-cells per square centimeter.

In an embodiment of the invention, the fold expansion of PBLs is from about 20% to about 100%, 25% to about 95%, 30% to about 90%, 35% to about 85%, 40% to about 80%, 45% to about 75%, 50% to about 100%, or 25% to about 75%. In an embodiment of the invention, the fold expansion is about 25%. In another embodiment of the invention, the fold expansion is about 50%. In another embodiment, the fold expansion is about 75%.

In an embodiment of the invention, additional IL-2 may be added to the culture on one or more days throughout the process. In an embodiment of the invention, additional IL-2 is added on Day 4. In an embodiment of the invention, additional IL-2 is added on Day 7. In an embodiment of the invention, additional IL-2 is added on Day 11. In another embodiment, additional IL-2 is added on Day 4, Day 7, and/or Day 11. In an embodiment of the invention, the cell culture medium may be changed on one or more days through the cell culture process. In an embodiment, the cell culture medium is changed on Day 4, Day 7, and/or Day 11 of the process. In an embodiment of the invention, the PBLs are cultured with additional IL-2 for a period of 1 day, 2 days, 3 days, 4 days, 5 days, 6 days, 7 days, 8 days, 9 days, 10 days, 11 days, 12 days, 13 days, or 14 days. In an embodiment of the invention, PBLs are cultured for a period of 3 days after each addition of IL-2.

In an embodiment, the cell culture medium is exchanged at least one time during the method. In an embodiment, the cell culture medium is exchanged at the same time that additional IL-2 is added. In another embodiment the cell culture medium is exchanged on at least one of Day 1, Day 2, Day 3, Day 4, Day 5, Day 6, Day 7, Day 8, Day 9, Day 10, Day 11, Day 12, Day 13, or Day 14. In an embodiment of the invention, the cell culture medium used throughout the method may be the same or different. In an embodiment of the invention, the cell culture medium is CM-2, CM-4, or AIM-V.

In an embodiment of the invention, T-cells may be restimulated with antiCD3/antiCD28 antibodies on one or more days throughout the 14-day expansion process. In an embodiment, the T-cells are restimulated on Day 7. In an embodiment, GRex 10M flasks are used for the restimulation step. In an embodiment of the invention, comparable flasks are used.

In an embodiment of the invention, the DynaBeads® are removed using a DynaMag™ Magnet, the cells are counted, and the cells are analyzed using phenotypic and functional analysis as further described in the Examples below. In an embodiment of the invention, antibodies are separated from the PBLs or MILs using methods known in the art. In any of the foregoing embodiments, magnetic bead-based selection of TILs, PBLs, or MILs is used.

In an embodiment of the invention, the PBMC sample is incubated for a period of time at a desired temperature effective to identify the non-adherent cells. In an embodiment of the invention, the incubation time is about 3 hours. In an embodiment of the invention, the temperature is about 37° Celsius. The non-adherent cells are then expanded using the process described above.

In an embodiment of the invention, the PBMCs are obtained from a patient who has been treated with ibrutinib or another ITK or kinase inhibitor, such ITK and kinase inhibitors as described elsewhere herein. In an embodiment of the invention, the ITK inhibitor is a covalent ITK inhibitor that covalently and irreversibly binds to ITK. In an embodiment of the invention, the ITK inhibitor is an allosteric ITK inhibitor that binds to ITK. In an embodiment of the invention, the PBMCs are obtained from a patient who has been treated with ibrutinib or other ITK inhibitor, including ITK inhibitors as described elsewhere herein, prior to obtaining a PBMC sample for use with any of the foregoing methods, including PBL Method 1. In an embodiment of the invention, the ITK inhibitor treatment has been administered at least 1 time, at least 2, times, or at least 3 times or more. In an embodiment of the invention, PBLs that are expanded from patients pretreated with ibrutinib or other ITK inhibitor comprise less LAG3+, PD-1+ cells than those expanded from patients not pretreated with ibrutinib or other ITK inhibitor. In an embodiment of the invention PBLs that are expanded from patients pretreated with ibrutinib or other ITK inhibitor comprise increased levels of IFNγ production than those expanded from patients not pretreated with ibrutinib or other ITK inhibitor. In an embodiment of the invention, PBLs that are expanded from patients pretreated with ibrutinib or other ITK inhibitor comprise increased lytic activity at lower Effector:Target cell ratios than those expanded from patients not pretreated with ibrutinib or other ITK inhibitor. In an embodiment of the invention, patients pretreated with ibrutinib or other ITK inhibitor have higher fold-expansion as compared with untreated patients.

In an embodiment of the invention, the method includes a step of adding an ITK inhibitor to the cell culture. In an embodiment, the ITK inhibitor is added on one or more of Day 0, Day 1, Day 2, Day 3, Day 4, Day 5, Day 6, Day 7, Day 8, Day 9, Day 10, Day 11, Day 12, Day 13, or Day 14 of the process. In an embodiment, the ITK inhibitor is added on the days during the method when cell culture medium is exchanged. In an embodiment, the ITK inhibitor is added on Day 0 and when cell culture medium is exchanged. In an embodiment, the ITK inhibitor is added during the method when IL-2 is added. In an embodiment, the ITK inhibitor is added on Day 0, Day 4, Day 7, and optionally Day 11 of the method. In an embodiment of the invention, the ITK inhibitor is added at Day 0 and at Day 7 of the method. In an embodiment of the invention, the ITK inhibitor is one known in the art. In an embodiment of the invention, the ITK inhibitor is one described elsewhere herein.

In an embodiment of the invention, the ITK inhibitor is used in the method at a concentration of from about 0.1 nM to about 5 uM. In an embodiment, the ITK inhibitor is used in the method at a concentration of about 0.1 nM, 0.5 nM, 1 nM, 5 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 uM, 2 uM, 3 uM, 4 uM, or 5 uM.

In an embodiment of the invention, the method includes a step of adding an ITK inhibitor when the PBMCs are derived from a patient who has no prior exposure to an ITK inhibitor treatment, such as ibrutinib.

In some embodiments, the PBMC sample is from a subject or patient who has been optionally pre-treated with a regimen comprising a kinase inhibitor or an ITK inhibitor. In some embodiments, the tumor sample is from a subject or patient who has been pre-treated with a regimen comprising a kinase inhibitor or an ITK inhibitor. In some embodiments, the PBMC sample is from a subject or patient who has been pre-treated with a regimen comprising a kinase inhibitor or an ITK inhibitor, has undergone treatment for at least 1 month, at least 2 months, at least 3 months, at least 4 months, at least 5 months, at least 6 months, or 1 year or more. In another embodiment, the PBMCs are derived from a patient who is currently on an ITK inhibitor regimen, such as ibrutinib.

In some embodiments, the PBMC sample is from a subject or patient who has been pre-treated with a regimen comprising a kinase inhibitor or an ITK inhibitor and is refractory to treatment with a kinase inhibitor or an ITK inhibitor, such as ibrutinib.

In some embodiments, the PBMC sample is from a subject or patient who has been pre-treated with a regimen comprising a kinase inhibitor or an ITK inhibitor but is no longer undergoing treatment with a kinase inhibitor or an ITK inhibitor. In some embodiments, the PBMC sample is from a subject or patient who has been pre-treated with a regimen comprising a kinase inhibitor or an ITK inhibitor but is no longer undergoing treatment with a kinase inhibitor or an ITK inhibitor and has not undergone treatment for at least 1 month, at least 2 months, at least 3 months, at least 4 months, at least 5 months, at least 6 months, or at least 1 year or more. In another embodiment, the PBMCs are derived from a patient who has prior exposure to an ITK inhibitor, but has not been treated in at least 3 months, at least 6 months, at least 9 months, or at least 1 year.

In an embodiment of the invention, at Day 0, cells are selected for CD19+ and sorted accordingly. In an embodiment of the invention, the selection is made using antibody binding beads. In an embodiment of the invention, pure T-cells are isolated on Day 0 from the PBMCs. In an embodiment of the invention, at Day 0, the CD19+B-cells and pure T-cells are co-cultured with antiCD3/antiCD28 antibodies for a minimum of 4 days. In an embodiment of the invention, on Day 4, IL-2 is added to the culture. In an embodiment of the invention, on Day 7, the culture is restimulated with antiCD3/antiCD28 antibodies and additional IL-2. In an embodiment of the invention, on Day 14, the PBLs are harvested.

In an embodiment of the invention, for patients that are not pre-treated with ibrutinib or other ITK inhibitor, 10-15 ml of Buffy Coat will yield about 5×10⁹ PBMC, which, in turn, will yield about 5.5×10⁷ starting cell material, and about 11×10⁹ PBLs at the end of the expansion process. In an embodiment of the invention, about 54×10⁶ PBMCs will yield about 6×10⁵ starting material, and about 1.2×10⁸ MTh (about a 205-fold expansion).

In an embodiment of the invention, for patients that are pre-treated with ibrutinib or other ITK inhibitor, the expansion process will yield about 20×10⁹ PBLs. In an embodiment of the invention, 40.3×10⁶ PBMCs will yield about 4.7×10⁵ starting cell material, and about 1.6×10⁸ PBLs (about a 338-fold expansion).

In an embodiment of the invention, the clinical dose of PBLs useful in the present invention for patients with chronic lymphocytic leukemia (CLL) is from about 0.1×10⁹ to about 15×10⁹ PBLs, from about 0.1×10⁹ to about 15×10⁹ PBLs, from about 0.12×10⁹ to about 12×10⁹ PBLs, from about 0.15×10⁹ to about 11×10⁹ PBLs, from about 0.2×10⁹ to about 10×109 PBLs, from about 0.3×10⁹ to about 9×10⁹ PBLs, from about 0.4×10⁹ to about 8×10⁹ PBLs, from about 0.5×10⁹ to about 7×10⁹ PBLs, from about 0.6×10⁹ to about 6×10⁹ PBLs, from about 0.7×10⁹ to about 5×10⁹ PBLs, from about 0.8×10⁹ to about 4×10⁹ PBLs, from about 0.9×10⁹ to about 3×109 PBLs, or from about 1×10⁹ to about 2×10⁹ PBLs.

In any of the foregoing embodiments, PBMCs may be derived from a whole blood sample, by apheresis, from the buffy coat, or from any other method known in the art for obtaining PBMCs.

In an embodiment, the invention provides a method for the preparation of peripheral blood lymphocytes (PBLs) comprising the steps of:
a. Obtaining a sample of peripheral blood mononuclear cells (PBMCs) from the peripheral blood of a patient, wherein said sample is optionally cryopreserved and the patient is optionally pretreated with an ITK inhibitor;
b. Optionally washing the PBMCs by centrifugation;
c. Admixing magnetic beads selective for CD3 and CD28 to the PBMCs to form an admixture of the beads and the PBMCs;
d. Seeding the admixture of the beads and the PBMCs into a gas-permeable container and co-culturing said PBMCs in media comprising about 3000 IU/mL of IL-2 in for about 4 to about 6 days;
e. Feeding said PBMCs using media comprising about 3000 IU/mL of IL-2, and co-culturing said PBMCs for about 5 days, such that the total co-culture period of steps d and e is about 9 to about 11 days;
f. Harvesting PBMCs from media;
g. Removing the magnetic beads selective for CD3 and CD28 from the harvested PBMCs using a magnet;
h. Removing residual B-cells from the harvested PBMCs using magnetic-activated cell sorting and magnetic beads selective for CD19 to provide a PBL product;
i. Washing and concentrating the PBL product using a cell harvester; and
j. Formulating and optionally cryopreserving the PBL product,
   wherein the ITK inhibitor is optionally an ITK inhibitor that covalently binds to ITK.

In an embodiment, the invention provides a method for the preparation of peripheral blood lymphocytes (PBLs) from a whole blood sample, the method comprising the steps of:
(a) obtaining peripheral blood mononuclear cells (PBMCs) from less than or equal to about 50 mL of whole blood from a patient having a liquid tumor, wherein the patient is optionally pretreated with an ITK inhibitor;
(b) admixing beads selective for CD3 and CD28 with the PBMCs, wherein the beads are added at a ratio of 3 beads:1 cell, to form an admixture of the PBMCs and the beads;
(c) culturing the admixture of the PBMCs and the beads at a density of about 25,000 cells per cm² to about 50,000 cells per cm² on a gas-permeable surface of one or more containers containing a first cell culture medium and IL-2 for a period of about 4 days;
(d) adding to each container of step (c) IL-2 and a second cell culture medium that is the same as or different from the first cell culture medium and culturing for a period of about 5 days to about 7 days to form an expanded population of PBLs; and
(e) harvesting from each container the expanded population of PBLs.

In an embodiment, the invention provides a method for the preparation of peripheral blood lymphocytes (PBLs) from a whole blood sample, the method comprising the steps of:
(a) obtaining peripheral blood mononuclear cells (PBMCs) from less than or equal to about 50 mL of whole blood from a patient having a liquid tumor, wherein the patient is optionally pretreated with an ITK inhibitor;
(b) removing B-cells from the PBMCs by selecting against CD19 to provide PBMCs depleted of B-cells;
(c) admixing beads selective for CD3 and CD28 with the PBMCs, wherein the beads are added at a ratio of 3 beads:1 cell, to form an admixture of the PBMCs and the beads;
(d) culturing the admixture of the PBMCs and the beads at a density of about 25,000 cells per cm² to about 50,000 cells per cm² on a gas-permeable surface of one or more containers containing a first cell culture medium and IL-2 for a period of about 4 days;
(e) adding to each container of step (d) IL-2 and a second cell culture medium that is the same as or different from the first cell culture medium and culturing for a period of about 5 days to about 7 days to form an expanded population of PBLs; and
(f) harvesting from each container the expanded population of PBLs.

In an embodiment, the invention provides a method for the preparation of peripheral blood lymphocytes (PBLs) from a whole blood sample, the method comprising the steps of:
(a) obtaining peripheral blood mononuclear cells (PBMCs) from less than or equal to about 50 mL of whole blood from a patient having a liquid tumor, wherein the patient is optionally pretreated with an ITK inhibitor;
(b) determining the proportion of the PMBCs constituted by B-cells as a B-cell percentage;
(c) if the B-cell percentage determined in step (b) is at least about seventy percent (70%), removing B-cells from the PBMCs by selecting against CD19 to provide PBMCs depleted of B-cells;
(d) admixing beads selective for CD3 and CD28 with the PBMCs, wherein the beads are added at a ratio of 3 beads:1 cell, to form an admixture of the PBMCs and the beads;
(e) culturing the admixture of the PBMCs and the beads at a density of about 25,000 cells per cm² to about 50,000 cells per cm² on a gas-permeable surface of one or more containers containing a first cell culture medium and IL-2 for a period of about 4 days;
(f) adding to each container of step (d) IL-2 and a second cell culture medium that is the same as or different from the first cell culture medium and culturing for a period of about 5 days to about 7 days to form an expanded population of PBLs; and
(g) harvesting from each container the expanded population of PBLs.

In an embodiment of the invention, removal of B-cells, or B-cell depletion (BCD), occurs on Day 0 or on Day 9 of a 9-day expansion process. In another embodiment, the BCD occurs on both Day 0 and Day 9 of a 9-day expansion process. In an embodiment of the invention, BCD occurs on Day 0 or Day 11 of an 11-day expansion process. In another embodiment, the BCD occurs on both Day 0 and Day 11 of an 11-day expansion process.

In an embodiment of the invention, the BCD step is performed on a PBMC sample from a patient having a high initial B-cell count. In one embodiment, a high initial B-cell count is about 50%, 55%, 60%, 65%, 70%, 75%, 80%, 85%, 90%, 95%, or more B-cells in the initial PBMC sample.

In an embodiment, the invention provides any of the methods described above modified as applicable such that if the B-cell percentage is at least about 70% the B-cell removal step, or BCD step, is performed.

In an embodiment, the invention provides any of the methods described above modified as applicable such that if the B-cell percentage is at least about 75% the B-cell removal step is performed.

In an embodiment, the invention provides any of the methods described above modified as applicable such that if the B-cell percentage is at least about 80% the B-cell removal step is performed.

In an embodiment, the invention provides any of the methods described above modified as applicable such that if the B-cell percentage is at least about 85% the B-cell removal step is performed.

In an embodiment, the invention provides any of the methods described above modified as applicable such that if the B-cell percentage is at least about 50%, 55%, 60%, 65%, 70%, 75%, 80%, 85%, 90%, 95%, or more the B-cell removal step is performed.

In an embodiment, the invention provides any of the methods described above modified as applicable such that the B-cell percentage is determined by comparison of the CD19+ cells to the CD45+ cells in the PBMCs.

In an embodiment, the invention provides any of the methods described above modified as applicable such that the B-cell percentage is determined by comparison of the fraction of CD19+/CD45+ cells to the fraction of CD45+ cells in the PBMCs.

In an embodiment, the invention provides any of the methods described above modified as applicable such that the comparison of the fraction of CD19+ cells to the fraction of CD45+ cells in the PBMCs is performed by contacting the PBMCs with a CD19 stain and a CD45 stain, and then comparing the subpopulation of PBMCs positive for the both CD19 stain and the CD45 stain with the subpopulation of PBMCs positive for only the CD19 stain.

In an embodiment, the invention provides any of the methods described above modified as applicable such that the CD19 stain is an anti-CD19 antibody conjugated to a first label and the CD45 stain is an anti-CD45 antibody conjugated to a second label.

In an embodiment, the invention provides any of the methods described above modified as applicable such that the first label is a first fluorochrome and the second label is a second fluorochrome that is different from the first fluorochrome.

In an embodiment, the invention provides any of the methods described above modified as applicable such that the total culturing period is from at or about 9 days to at or about 11 days.

In an embodiment, the invention provides any of the methods described above modified as applicable such that the total culturing period is at or about 9 days, at or about 10 days or at or about 11 days.

In an embodiment, the invention provides any of the methods described above modified as applicable such that the total culturing period is from at or about 9 days to at or about 14 days.

In an embodiment, the invention provides any of the methods described above modified as applicable such that the total culturing period is at or about 9 days, at or about 10 days, at or about 11 days, at or about 12 days, at or about 13 days, or at or at or about 14 days.

In an embodiment, the invention provides any of the methods described above modified as applicable such that the PBMCs are obtained from at or about 50 mL of peripheral blood of the patient.

In an embodiment, the invention provides any of the methods described above modified as applicable such that the PBMCs are obtained from at or about 10 mL to at or about 50 mL of peripheral blood of the patient.

In an embodiment, the invention provides any of the methods described above modified as applicable such that the PBMCs are obtained from at or about 10 mL, at or about 20 mL, at or about 30 mL, at or about 40 mL, or at or about 50 mL of peripheral blood of the patient.

In an embodiment, the invention provides any of the methods described above modified as applicable such that the PBMCs are obtained from at or about 10 mL to at or about 100 mL of peripheral blood of the patient

In an embodiment, the invention provides any of the methods described above modified as applicable such that the PBMCs are obtained from at or about 10 mL, at or about 20 mL, at or about 30 mL, at or about 40 mL, at or about 50 mL, at or about 60 mL, at or about 70 mL, at or about 80 mL, at or about 90 mL, or at or about 100 mL of peripheral blood of the patient.

In an embodiment, the invention provides any of the methods described above modified as applicable such that the total number of cells harvested is from at or about 1 billion to at or about 8 billion.

In an embodiment, the invention provides any of the methods described above modified as applicable such that the total number of cells harvested is from at or about 1 billion, about 2 billion, about 3 billion, about 4 billion, about 5 billion, and 6 billion, about 7 billion, about 8 billion, about 9 billion, or about 10 billion.

In an embodiment, the invention provides any of the methods described above modified as applicable such that the total number of cells harvested is from at or about 8 billion to at or about 22 billion.

In an embodiment, the invention provides any of the methods described above modified as applicable such that the total number of cells harvested is from at or about 2 billion to at or about 50 billion.

In an embodiment, the invention provides any of the methods described above modified as applicable such that the total number of cells harvested is from at or about 8 billion, at or about 9 billion, at or about 10 billion, at or about 11 billion, at or about 12 billion, at or about 13 billion, at or about 14 billion, at or about 15 billion, at or about 16 billion, at or about 17 billion, at or about 18 billion, at or about 19 billion, at or about 20 billion, at or about 21 billion, or at or about 22 billion.

In an embodiment, the invention provides any of the methods described above modified as applicable such that the PBMCs are cultured in a plurality of gas-permeable containers.

In an embodiment, the invention provides any of the methods described above modified as applicable such that the PBMCs are cultured in at least two gas-permeable containers.

In an embodiment, the invention provides any of the methods described above modified as applicable such that the PBMCs are cultured in at least five gas-permeable containers.

In an embodiment, the invention provides any of the methods described above modified as applicable such that the PBMCs are cultured in 2 to 20 gas-permeable containers.

In an embodiment, the invention provides any of the methods described above modified as applicable such that the PBMCs are cultured in up to 5 gas-permeable containers.

In an embodiment, the invention provides any of the methods described above modified as applicable such that the PBMCs are cultured in 2, 3, 4, 5, 6, 7, 8, 9, 10, 11, 12, 13, 14, 15, 16, 17, 18, 19 or 20 gas-permeable containers.

In an embodiment, the invention provides any of the methods described above modified as applicable such that the PBMCs are seeded at a density of at or about 12,500 cells per cm² to at or about 50,000 cells per cm² in each gas-permeable container.

In an embodiment, the invention provides any of the methods described above modified as applicable such that the PBMCs are seeded at a density of at or about 6,250 cells per cm² to at or about 25,000 cells per cm² in each gas-permeable container.

In an embodiment, the invention provides any of the methods described above modified as applicable such that the PBMCs are seeded at a density of at or about 6,250 cells per cm² to at or about 50,000 cells per cm² in each gas-permeable container.

In an embodiment, the invention provides any of the methods described above modified as applicable such that the PBMCs are seeded at a density of at or about 25,000 cells per cm² to at or about 50,000 cells per cm² in each gas-permeable container.

In an embodiment, the invention provides any of the methods described above modified as applicable such that the PBMCs are seeded at a density of at or about 6,250 cells per cm², at or about 9,375 cells per cm², at or about 12,500 cells per cm², at or about 15,625 cells per cm², at or about 18,750 cells per cm², at or about 21,875 cells per cm², at or about 25,000 cells per cm², at or about 28,125 cells per cm², at or about 31,250 cells per cm², at or about 34,375 cells per cm², at or about 37,500 cells per cm², at or about 40,625 cells per cm², at or about 43,750 cells per cm², at or about 47,875 cells per cm², or at or about at or about 50,000 cells per cm² in each gas-permeable container.

In an embodiment, the invention provides any of the methods described above modified as applicable such that the step of admixing the beads selective for CD3 and CD28 with the PBMCs to form an admixture of the beads and the PBMCs is replaced with the step of admixing the beads selective for CD3 and CD28 with the PBMCs to form complexes of the beads and the PBMCs in an admixture of the beads and the PBMCs, and wherein the step of culturing the admixture is replaced with the step of separating the complexes of the beads and the PBMCs from the admixture and culturing the complexes of PBMCs and the beads at a density of about 25,000 cells per cm² to about 50,000 cells per cm² on a gas-permeable surface in one or more containers containing a first cell culture medium and IL-2 for a period of about 4 days. In another embodiment, the beads selective for CD3 and CD28 are magnetic beads, and the step of separating the complexes of the beads and the PBMCs from the admixture is performed by using a magnet to remove the complexes from the admixture.

In an embodiment, the invention provides any of the methods described above modified as applicable such that the beads selective for CD3 and CD28 are beads conjugated to anti-CD3 antibodies and anti-CD28 antibodies.

In an embodiment, the invention provides any of the methods described above modified as applicable such that the removal of B-cells from the PBMCs is performed by contacting PBMCs with beads selective for CD19 to form bead-CD19+ cell complexes and removing the complexes to provide PBMCs depleted of B-cells. In another embodiment, the beads selective for CD19 are magnetic beads and a magnet is used to remove magnetic bead-CD19+ cell complexes from the PBMCs. In another embodiment, the beads selective for CD19 are beads conjugated to anti-CD19 antibodies. In another embodiment, the beads conjugated to anti-CD19 antibodies are CliniMACS™ anti-CD19 beads (Miltenyi).

In an embodiment, the invention provides any of the methods described above modified as applicable such that after the step of harvesting the expanded population of PBLs the method comprises the step of performing a selection to remove any remnant B-cells from the expanded population of PBLs.

In an embodiment, the invention provides any of the methods described above modified as applicable such that the selection to remove any remnant B-cells from the expanded population of PBLs is performed by admixing beads selective for CD19 with the expanded population of PBLs to form complexes of beads and any remnant B-cells and removing the complexes from the expanded population of PBLs.

In an embodiment, the invention provides any of the methods described above modified as applicable such that the selection to remove any remnant B-cells from the expanded population of PBLs is performed by admixing magnetic beads selective for CD19 with the expanded population of PBLs to form complexes of magnetic beads and any remnant B-cells and using a magnet to remove the complexes from the expanded population of PBLs.

In an embodiment, the invention provides any of the methods described above modified as applicable such that the beads selective for CD19 are beads conjugated to anti-CD19 antibody.

In an embodiment, the invention provides any of the methods described above modified as applicable such that the first cell culture medium contains about 3000 IU/mL of IL-2.

In an embodiment, the invention provides any of the methods described above modified as applicable such that the second cell culture medium contains about 3000 IU/mL of IL-2.

In an embodiment, the invention provides any of the methods described above modified as applicable such that the cultures in the culturing steps are incubated at 37° C. and under an atmosphere containing 5% CO₂.

In an embodiment, the invention provides any of the methods described above modified as applicable such that the patient is pretreated with an ITK inhibitor.

In an embodiment, the invention provides any of the methods described above modified as applicable such that the patient is pretreated with an ITK inhibitor and is refractory to treatment with the ITK inhibitor.

In an embodiment, the invention provides any of the methods described above modified as applicable such that the patient is pretreated with ibrutinib.

In an embodiment, the invention provides any of the methods described above modified as applicable such that the patient is suffering from a leukemia.

In an embodiment, the invention provides any of the methods described above modified as applicable such that the patient is suffering from a chronic lymphocytic leukemia.

### Pharmaceutical Compositions, Dosages, and Dosing Regimens for PBLs

In another embodiment, the invention provides a therapeutic population of PBLs prepared by any method of expanding PBLs described herein, optionally modified to express a chimeric antigen receptor (CAR) and/or express a modified T-cell receptor and/or suppress or reduce expression of one or more immune checkpoint genes as described herein.

In another embodiment, the invention provides a pharmaceutical composition comprising a therapeutic population of PBLs prepared by any method of expanding PBLs described herein, optionally modified to express a chimeric antigen receptor (CAR) and/or express a modified T-cell receptor and/or suppress or reduce expression of one or more immune checkpoint genes as described herein, and a pharmaceutically acceptable carrier.

In an embodiment, PBLs expanded using methods of the present disclosure are administered to a patient as a pharmaceutical composition. In an embodiment, the pharmaceutical composition is a suspension of PBLs in a sterile buffer. PBLs expanded using methods of the present disclosure may be administered by any suitable route as known in the art. Preferably, the PBLs are administered as a single intra-arterial or intravenous infusion, which preferably lasts approximately 30 to 60 minutes. Other suitable routes of administration include intraperitoneal, intrathecal, and intralymphatic administration.

Any suitable dose of PBLs can be administered. Preferably, from about 2.3×10¹⁰ to about 13.7×10¹⁰ PBLs are administered, with an average of around 7.8×10¹⁰ PBLs, particularly if the cancer is a hematological malignancy. In an embodiment, about 1.2×10¹⁰ to about 4.3×10¹⁰ of PBLs are administered. In an embodiment, about 8 billion to about 22 billion PBLs are administered.

In some embodiments, the number of the PBLs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In an embodiment, the number of the PBLs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³. In an embodiment of the invention, the number of PBLs provided in the pharmaceutical compositions of the invention is in the range of from about 4×10⁸ to about 2.5×10⁹. In another embodiment, the number of PBLs provided in the pharmaceutical compositions of the invention is 9.5×10⁸. In another embodiment, the number of PBLs provided in the pharmaceutical compositions of the invention is 4.1×10⁸. In another embodiment, the number of PBLs provided in the pharmaceutical compositions of the invention is 2.2×10⁹.

In an embodiment of the invention, the number of PBLs provided in the pharmaceutical compositions of the invention is in the range of from about 0.1×10⁹ to about 15×10⁹ PBLs, from about 0.1×10⁹ to about 15×10⁹ PBLs, from about 0.12×10⁹ to about 12×10⁹ PBLs, from about 0.15×10⁹ to about 11×10⁹ PBLs, from about 0.2×10⁹ to about 10×10⁹ PBLs, from about 0.3×10⁹ to about 9×10⁹ PBLs, from about 0.4×10⁹ to about 8×10⁹ PBLs, from about 0.5×10⁹ to about 7×10⁹ PBLs, from about 0.6×10⁹ to about 6×10⁹ PBLs, from about 0.7×10⁹ to about 5×10⁹ PBLs, from about 0.8×10⁹ to about 4×10⁹ PBLs, from about 0.9×10⁹ to about 3×10⁹ PBLs, or from about 1×10⁹ to about 2×10⁹ PBLs.

In some embodiments, the concentration of the PBLs provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of the PBLs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of the PBLs provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the PBLs provided in the pharmaceutical compositions of the invention is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of the PBLs provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the PBLs provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

The PBLs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the PBLs may also be used if appropriate. The amounts of the pharmaceutical compositions administered using the methods herein, such as the dosages of PBLs, will be dependent on the human or mammal being treated, the severity of the disorder or condition, the rate of administration, the disposition of the active pharmaceutical ingredients and the discretion of the prescribing physician.

In some embodiments, PBLs may be administered in a single dose. Such administration may be by injection, e.g., intravenous injection. In some embodiments, PBLs may be administered in multiple doses. Dosing may be once, twice, three times, four times, five times, six times, or more than six times per year. Dosing may be once a month, once every two weeks, once a week, or once every other day. Administration of PBLs may continue as long as necessary.

In some embodiments, an effective dosage of PBLs is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, an effective dosage of PBLs is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, an effective dosage of PBLs is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of PBLs is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg.

An effective amount of the PBLs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation or direct injection into tumor, or by inhalation.

In some embodiments, the invention provides the pharmaceutical composition described in any of the preceding paragraphs as applicable above modified such that the pharmaceutical composition comprises 1.5×10⁸ to 20×10⁹ PBLs.

In some embodiments, the invention provides the pharmaceutical composition described in any of the preceding paragraphs as applicable above modified such that the pharmaceutical composition further comprises a cryopreservant.

In some embodiments, the invention provides the pharmaceutical composition described in any of the preceding paragraphs as applicable above modified such that the pharmaceutical composition further comprises at or about 5% (v/v) dimethylsulfoxide (DMSO).

In some embodiments, the invention provides the pharmaceutical composition described in any of the preceding paragraphs as applicable above modified such that the pharmaceutical composition further comprises at or about 50% (v/v) CryoStor® CS10 cryopreservation medium.

In some embodiments, the invention provides the pharmaceutical composition described in any of the preceding paragraphs as applicable above modified such that the pharmaceutical composition further comprises at or about 50% (v/v) CryoStor® CS10 cryopreservation medium and at or about 5% (v/v) DMSO.

In some embodiments, the invention provides the pharmaceutical composition described in any of the preceding paragraphs as applicable above modified such that the pharmaceutical composition further comprises a stabilizer.

In some embodiments, the invention provides the pharmaceutical composition described in any of the preceding paragraphs as applicable above modified such that the pharmaceutical composition further comprises at or about 0.5% (w/v) human serum albumin (HSA).

In some embodiments, the invention provides the pharmaceutical composition described in any of the preceding paragraphs as applicable above modified such that the pharmaceutical composition further comprises an isotonic agent.

In some embodiments, the invention provides the pharmaceutical composition described in any of the preceding paragraphs as applicable above modified such that the pharmaceutical composition further comprises at or about 50% (v/v) Plasma-Lyte A.

In some embodiments, the invention provides the pharmaceutical composition described in any of the preceding paragraphs as applicable above modified such that the pharmaceutical composition further comprises at or about 300 IU/mL of IL-2.

In some embodiments, the invention provides the pharmaceutical composition described in any of the preceding paragraphs as applicable above modified such that the pharmaceutical composition further comprises at or about 50% (v/v) CryoStor® CS10 cryopreservation medium, at or about 5% (v/v) DMSO, at or about 0.5% (w/v) human serum albumin (HSA), at or about 50% (v/v) Plasma-Lyte A, and at or about 300 IU/mL of IL-2.

In some embodiments, the invention provides the pharmaceutical composition described in any of the preceding paragraphs as applicable above modified such that the pharmaceutical composition comprises 1.5×10⁸ to 20×10⁹ PBLs and further comprises at or about 50% (v/v) CryoStor® CS10 cryopreservation medium, at or about 5% (v/v) DMSO, at or about 0.5% (w/v) human serum albumin (HSA), at or about 50% (v/v) Plasma-Lyte A, and at or about 300 IU/mL of IL-2.

### Optional Genetic Engineering of PBLs

In some embodiments, the expanded PBLs of the present invention are further manipulated before, during, or after an expansion step, including during closed, sterile manufacturing processes, each as provided herein, in order to alter protein expression in a transient manner. In some embodiments, the transiently altered protein expression is due to transient gene editing. In some embodiments, the expanded PBLs of the present invention are treated with transcription factors (TFs) and/or other molecules capable of transiently altering protein expression in the PBLs. In some embodiments, the TFs and/or other molecules that are capable of transiently altering protein expression provide for altered expression of tumor antigens and/or an alteration in the number of tumor antigen-specific T cells in a population of PBLs.

In certain embodiments, the method comprises genetically editing a population of PBLs. In certain embodiments, the method comprises genetically editing a population of PBLs provided at different stages of any of the processes described herein.

In some embodiments, the present invention includes genetic editing through nucleotide insertion, such as through ribonucleic acid (RNA) insertion, including insertion of messenger RNA (mRNA) or small (or short) interfering RNA (siRNA), into a population of PBLs for promotion of the expression of one or more proteins or inhibition of the expression of one or more proteins, as well as simultaneous combinations of both promotion of one set of proteins with inhibition of another set of proteins.

In some embodiments, the expanded PBLs of the present invention undergo transient alteration of protein expression. In some embodiments, the transient alteration of protein expression occurs at any time before, during, or after the expansion process. In some embodiments, the transient alteration of protein expression occurs at any step within the expansion process. In some embodiments, the transient alteration of protein expression occurs in the bulk PBL population prior to a first expansion. In some embodiments, the transient alteration of protein expression occurs during the first expansion. In some embodiments, the transient alteration of protein expression occurs after the first expansion, including, for example in the PBL population in transition between the first and second expansion (e.g. the second population of PBLs as described herein. In some embodiments, the transient alteration of protein expression occurs in the bulk PBL population prior to second expansion. In some embodiments, the transient alteration of protein expression occurs during the second expansion, including, for example in the PBL population being expanded (e.g. the third population of PBLs). In some embodiments, the transient alteration of protein expression occurs after the second expansion.

In an embodiment, a method of transiently altering protein expression in a population of PBLs includes the step of electroporation. Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. In an embodiment, a method of transiently altering protein expression in population of PBLs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In an embodiment, a method of transiently altering protein expression in a population of PBLs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In an embodiment, a method of transiently altering protein expression in a population of PBLs includes the step of transfection using methods described in U.S. Pat. Nos. 5,766,902; 6,025,337; 6,410,517; 6,475,994; and 7,189,705; the disclosures of each of which are incorporated by reference herein.

In some embodiments, transient alteration of protein expression results in an increase in Stem Memory T cells (TSCMs). TSCMs are early progenitors of antigen-experienced central memory T cells. TSCMs generally display the long-term survival, self-renewal, and multipotency abilities that define stem cells, and are generally desirable for the generation of effective TIL products. TSCM have shown enhanced anti-tumor activity compared with other T cell subsets in mouse models of adoptive cell transfer (Gattinoni et al. Nat Med 2009, 2011; Gattinoni, Nature Rev. Cancer, 2012; Cieri et al. Blood 2013). In some embodiments, transient alteration of protein expression results in a TIL population with a composition comprising a high proportion of TSCM. In some embodiments, transient alteration of protein expression results in an at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% increase in TSCM percentage. In some embodiments, transient alteration of protein expression results in an at least a 1-fold, 2-fold, 3-fold, 4-fold, 5-fold, or 10-fold increase in TSCMs in the TIL population. In some embodiments, transient alteration of protein expression results in a TIL population with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% TSCMs. In some embodiments, transient alteration of protein expression results in a therapeutic TIL population with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% TSCMs.

In some embodiments, transient alteration of protein expression results in rejuvenation of antigen-experienced T-cells. In some embodiments, rejuvenation includes, for example, increased proliferation, increased T-cell activation, and/or increased antigen recognition.

In some embodiments, transient alteration of protein expression alters the expression in a large fraction of the T-cells in order to preserve the tumor-derived TCR repertoire. In some embodiments, transient alteration of protein expression does not alter the tumor-derived TCR repertoire. In some embodiments, transient alteration of protein expression maintains the tumor-derived TCR repertoire.

In some embodiments, transient alteration of protein results in altered expression of a particular gene. In some embodiments, the transient alteration of protein expression targets a gene including but not limited to PD-1 (also referred to as PDCD1 or CC279), TGFBR2, CCR4/5, CBLB (CBL-B), CISH, CCRs (chimeric co-stimulatory receptors), IL-2, IL-12, IL-15, IL-21, NOTCH 1/2 ICD, TIM3, LAG3, TIGIT, TGFβ, CCR2, CCR4, CCR5, CXCR1, CXCR2, CSCR3, CCL2 (MCP-1), CCL3 (MIP-1α), CCL4 (MIP1-β), CCL5 (RANTES), CXCL1/CXCL8, CCL22, CCL17, CXCL1/CXCL8, VHL, CD44, PIK3CD, SOCS1, and/or cAMP protein kinase A (PKA). In some embodiments, the transient alteration of protein expression targets a gene selected from the group consisting of PD-1, TGFBR2, CCR4/5, CBLB (CBL-B), CISH, CCRs (chimeric co-stimulatory receptors), IL-2, IL-12, IL-15, IL-21, NOTCH 1/2 ICD, TIM3, LAG3, TIGIT, TGFβ, CCR2, CCR4, CCR5, CXCR1, CXCR2, CSCR3, CCL2 (MCP-1), CCL3 (MIP-1α), CCL4 (MIP1-β), CCL5 (RANTES), CXCL1/CXCL8, CCL22, CCL17, CXCL1/CXCL8, VHL, CD44, PIK3CD, SOCS1, and/or cAMP protein kinase A (PKA). In some embodiments, the transient alteration of protein expression targets PD-1. In some embodiments, the transient alteration of protein expression targets TGFBR2. In some embodiments, the transient alteration of protein expression targets CCR4/5. In some embodiments, the transient alteration of protein expression targets CBLB. In some embodiments, the transient alteration of protein expression targets CISH. In some embodiments, the transient alteration of protein expression targets CCRs (chimeric co-stimulatory receptors). In some embodiments, the transient alteration of protein expression targets IL-2. In some embodiments, the transient alteration of protein expression targets IL-12. In some embodiments, the transient alteration of protein expression targets IL-15. In some embodiments, the transient alteration of protein expression targets IL-21. In some embodiments, the transient alteration of protein expression targets NOTCH 1/2 ICD. In some embodiments, the transient alteration of protein expression targets TIM3. In some embodiments, the transient alteration of protein expression targets LAG3. In some embodiments, the transient alteration of protein expression targets TIGIT. In some embodiments, the transient alteration of protein expression targets TGFβ. In some embodiments, the transient alteration of protein expression targets CCR1. In some embodiments, the transient alteration of protein expression targets CCR2. In some embodiments, the transient alteration of protein expression targets CCR4. In some embodiments, the transient alteration of protein expression targets CCR5. In some embodiments, the transient alteration of protein expression targets CXCR1. In some embodiments, the transient alteration of protein expression targets CXCR2. In some embodiments, the transient alteration of protein expression targets CSCR3. In some embodiments, the transient alteration of protein expression targets CCL2 (MCP-1). In some embodiments, the transient alteration of protein expression targets CCL3 (MIP-1α). In some embodiments, the transient alteration of protein expression targets CCL4 (MIP1-β). In some embodiments, the transient alteration of protein expression targets CCL5 (RANTES). In some embodiments, the transient alteration of protein expression targets CXCL1. In some embodiments, the transient alteration of protein expression targets CXCL8. In some embodiments, the transient alteration of protein expression targets CCL22. In some embodiments, the transient alteration of protein expression targets CCL17. In some embodiments, the transient alteration of protein expression targets VHL. In some embodiments, the transient alteration of protein expression targets CD44. In some embodiments, the transient alteration of protein expression targets PIK3CD. In some embodiments, the transient alteration of protein expression targets SOCS1. In some embodiments, the transient alteration of protein expression targets cAMP protein kinase A (PKA).

In some embodiments, the transient alteration of protein expression results in increased and/or overexpression of a chemokine receptor. In some embodiments, the chemokine receptor that is overexpressed by transient protein expression includes a receptor with a ligand that includes but is not limited to CCL2 (MCP-1), CCL3 (MIP-1α), CCL4 (MIP1-β), CCL5 (RANTES), CXCL1, CXCL8, CCL22, and/or CCL17.

In some embodiments, the transient alteration of protein expression results in a decrease and/or reduced expression of PD-1, CTLA-4, TIM-3, LAG-3, TIGIT, TGFβR2, and/or TGFβ (including resulting in, for example, TGFβ pathway blockade). In some embodiments, the transient alteration of protein expression results in a decrease and/or reduced expression of CBLB (CBL-B). In some embodiments, the transient alteration of protein expression results in a decrease and/or reduced expression of CISH.

In some embodiments, the transient alteration of protein expression results in increased and/or overexpression of chemokine receptors in order to, for example, improve TIL trafficking or movement to the tumor site. In some embodiments, the transient alteration of protein expression results in increased and/or overexpression of a CCR (chimeric co-stimulatory receptor). In some embodiments, the transient alteration of protein expression results in increased and/or overexpression of a chemokine receptor selected from the group consisting of CCR1, CCR2, CCR4, CCR5, CXCR1, CXCR2, and/or CSCR3.

In some embodiments, the transient alteration of protein expression results in increased and/or overexpression of an interleukin. In some embodiments, the transient alteration of protein expression results in increased and/or overexpression of an interleukin selected from the group consisting of IL-2, IL-12, IL-15, and/or IL-21.

In some embodiments, the transient alteration of protein expression results in increased and/or overexpression of NOTCH 1/2 ICD. In some embodiments, the transient alteration of protein expression results in increased and/or overexpression of VHL. In some embodiments, the transient alteration of protein expression results in increased and/or overexpression of CD44. In some embodiments, the transient alteration of protein expression results in increased and/or overexpression of PIK3CD. In some embodiments, the transient alteration of protein expression results in increased and/or overexpression of SOCS1,

In some embodiments, the transient alteration of protein expression results in decreased and/or reduced expression of cAMP protein kinase A (PKA).

In some embodiments, the transient alteration of protein expression results in decreased and/or reduced expression of a molecule selected from the group consisting of PD-1, LAG3, TIM3, CTLA-4, TIGIT, CISH, TGFβR2, PKA, CBLB, BAFF (BR3), and combinations thereof. In some embodiments, the transient alteration of protein expression results in decreased and/or reduced expression of two molecules selected from the group consisting of PD-1, LAG3, CTLA-4, TIGIT, CISH, TGFβR2, PKA, CBLB, BAFF (BR3), and combinations thereof. In some embodiments, the transient alteration of protein expression results in decreased and/or reduced expression of PD-1 and one molecule selected from the group consisting of LAG3, CTLA-4, TIGIT, CISH, TGFβR2, PKA, CBLB, BAFF (BR3), and combinations thereof. In some embodiments, the transient alteration of protein expression results in decreased and/or reduced expression of PD-1, LAG-3, CISH, CBLB, TIM3, and combinations thereof. In some embodiments, the transient alteration of protein expression results in decreased and/or reduced expression of PD-1 and one of LAG3, CISH, CBLB, TIM3, and combinations thereof. In some embodiments, the transient alteration of protein expression results in decreased and/or reduced expression of PD-1 and LAG3. In some embodiments, the transient alteration of protein expression results in decreased and/or reduced expression of PD-1 and CISH. In some embodiments, the transient alteration of protein expression results in decreased and/or reduced expression of PD-1 and CBLB. In some embodiments, the transient alteration of protein expression results in decreased and/or reduced expression of LAG3 and CISH. In some embodiments, the transient alteration of protein expression results in decreased and/or reduced expression of LAG3 and CBLB. In some embodiments, the transient alteration of protein expression results in decreased and/or reduced expression of CISH and CBLB. In some embodiments, the transient alteration of protein expression results in decreased and/or reduced expression of TIM3 and PD-1. In some embodiments, the transient alteration of protein expression results in decreased and/or reduced expression of TIM3 and LAG3. In some embodiments, the transient alteration of protein expression results in decreased and/or reduced expression of TIM3 and CISH. In some embodiments, the transient alteration of protein expression results in decreased and/or reduced expression of TIM3 and CBLB.

In some embodiments, an adhesion molecule selected from the group consisting of CCR2, CCR4, CCR5, CXCR2, CXCR3, CX3CR1, and combinations thereof, is inserted by a gammaretroviral or lentiviral method into the first population of PBLs, second population of PBLs, or harvested population of PBLs (e.g., the expression of the adhesion molecule is increased).

In some embodiments, the transient alteration of protein expression results in decreased and/or reduced expression of a molecule selected from the group consisting of PD-1, LAG3, TIM3, CTLA-4, TIGIT, CISH, TGFβR2, PKA, CBLB, BAFF (BR3), and combinations thereof, and increased and/or enhanced expression of CCR2, CCR4, CCR5, CXCR2, CXCR3, CX3CR1, and combinations thereof. In some embodiments, the transient alteration of protein expression results in decreased and/or reduced expression of a molecule selected from the group consisting of PD-1, LAG3, TIM3, CISH, CBLB, and combinations thereof, and increased and/or enhanced expression of CCR2, CCR4, CCR5, CXCR2, CXCR3, CX3CR1, and combinations thereof.

In some embodiments, there is a reduction in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is a reduction in expression of at least about 65%, about 70%, about 75%, about 80%, about 85%, about 90%, or about 95%. In some embodiments, there is a reduction in expression of at least about 75%, about 80%, about 85%, about 90%, or about 95%. In some embodiments, there is a reduction in expression of at least about 80%, about 85%, about 90%, or about 95%. In some embodiments, there is a reduction in expression of at least about 85%, about 90%, or about 95%. In some embodiments, there is a reduction in expression of at least about 80%. In some embodiments, there is a reduction in expression of at least about 85%, In some embodiments, there is a reduction in expression of at least about 90%. In some embodiments, there is a reduction in expression of at least about 95%. In some embodiments, there is a reduction in expression of at least about 99%.

In some embodiments, there is an increase in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is an increase in expression of at least about 65%, about 70%, about 75%, about 80%, about 85%, about 90%, or about 95%. In some embodiments, there is an increase in expression of at least about 75%, about 80%, about 85%, about 90%, or about 95%. In some embodiments, there is an increase in expression of at least about 80%, about 85%, about 90%, or about 95%. In some embodiments, there is an increase in expression of at least about 85%, about 90%, or about 95%. In some embodiments, there is an increase in expression of at least about 80%. In some embodiments, there is an increase in expression of at least about 85%, In some embodiments, there is an increase in expression of at least about 90%. In some embodiments, there is an increase in expression of at least about 95%. In some embodiments, there is an increase in expression of at least about 99%.

In some embodiments, transient alteration of protein expression is induced by treatment of the PBLs with transcription factors (TFs) and/or other molecules capable of transiently altering protein expression in the PBLs. In some embodiments, the SQZ vector-free microfluidic platform is employed for intracellular delivery of the transcription factors (TFs) and/or other molecules capable of transiently altering protein expression. Such methods demonstrating the ability to deliver proteins, including transcription factors, to a variety of primary human cells, including T cells (Sharei et al. PNAS 2013, as well as Sharei et al. PLOS ONE 2015 and Greisbeck et al. J. Immunology vol. 195, 2015) have been described; see, for example, International Patent Publications WO 2013/059343A1, WO 2017/008063A1, and WO 2017/123663A1, all of which are incorporated by reference herein in their entireties. Such methods as described in International Patent Publications WO 2013/059343A1, WO 2017/008063A1, and WO 2017/123663A1 can be employed with the present invention in order to expose a population of PBLs to transcription factors (TFs) and/or other molecules capable of inducing transient protein expression, wherein said TFs and/or other molecules capable of inducing transient protein expression provide for increased expression of tumor antigens and/or an increase in the number of tumor antigen-specific T cells in the population of PBLs, thus resulting in reprogramming of the TIL population and an increase in therapeutic efficacy of the reprogrammed TIL population as compared to a non-reprogrammed TIL population. In some embodiments, the reprogramming results in an increased subpopulation of effector T cells and/or central memory T cells relative to the starting or prior population (i.e., prior to reprogramming) population of PBLs, as described herein.

In some embodiments, the transcription factor (TF) includes but is not limited to TCF-1, NOTCH 1/2 ICD, and/or MYB. In some embodiments, the transcription factor (TF) is TCF-1. In some embodiments, the transcription factor (TF) is NOTCH 1/2 ICD. In some embodiments, the transcription factor (TF) is MYB. In some embodiments, the transcription factor (TF) is administered with induced pluripotent stem cell culture (iPSC), such as the commercially available KNOCKOUT Serum Replacement (Gibco/ThermoFisher), to induce additional TIL reprogramming. In some embodiments, the transcription factor (TF) is administered with an iPSC cocktail to induce additional TIL reprogramming. In some embodiments, the transcription factor (TF) is administered without an iPSC cocktail. In some embodiments, reprogramming results in an increase in the percentage of TSCMs. In some embodiments, reprogramming results in an increase in the percentage of TSCMs by about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95% TSCMs.

In some embodiments, a method of transient altering protein expression, as described above, may be combined with a method of genetically modifying a population of PBLs by including the step of stable incorporation of genes for production of one or more proteins. In certain embodiments, the method comprises a step of genetically modifying a population of PBLs. In certain embodiments, the method comprises genetically modifying the first population of PBLs, the second population of PBLs and/or the third population of PBLs. In an embodiment, a method of genetically modifying a population of PBLs includes the step of retroviral transduction. In an embodiment, a method of genetically modifying a population of PBLs includes the step of lentiviral transduction. Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein. In an embodiment, a method of genetically modifying a population of PBLs includes the step of gamma-retroviral transduction. Gamma-retroviral transduction systems are known in the art and are described, e.g., Cepko and Pear, Cur. Prot. Mol. Biol. 1996, 9.9.1-9.9.16, the disclosure of which is incorporated by reference herein. In an embodiment, a method of genetically modifying a population of PBLs includes the step of transposon-mediated gene transfer. Transposon-mediated gene transfer systems are known in the art and include systems wherein the transposase is provided as DNA expression vector or as an expressible RNA or a protein such that long-term expression of the transposase does not occur in the transgenic cells, for example, a transposase provided as an mRNA (e.g., an mRNA comprising a cap and poly-A tail). Suitable transposon-mediated gene transfer systems, including the salmonid-type Tel-like transposase (SB or Sleeping Beauty transposase), such as SB10, SB11, and SB100x, and engineered enzymes with increased enzymatic activity, are described in, e.g., Hackett, et al., Mol. Therapy 2010, 18, 674-83 and U.S. Pat. No. 6,489,458, the disclosures of each of which are incorporated by reference herein.

In some embodiments, transient alteration of protein expression is a reduction in expression induced by self-delivering RNA interference (sdRNA), which is a chemically-synthesized asymmetric siRNA duplex with a high percentage of 2′-OH substitutions (typically fluorine or —OCH₃) which comprises a 20-nucleotide antisense (guide) strand and a 13 to 15 base sense (passenger) strand conjugated to cholesterol at its 3′ end using a tetraethylenglycol (TEG) linker. In some embodiments, the method comprises transient alteration of protein expression in a population of PBLs, comprising the use of self-delivering RNA interference (sdRNA), which is a chemically-synthesized asymmetric siRNA duplex with a high percentage of 2′-OH substitutions (typically fluorine or —OCH₃) which comprises a 20-nucleotide antisense (guide) strand and a 13 to 15 base sense (passenger) strand conjugated to cholesterol at its 3′ end using a tetraethylenglycol (TEG) linker. Methods of using sdRNA have been described in Khvorova and Watts, Nat. Biotechnol. 2017, 35, 238-248; Byrne, et al., J. Ocul. Pharmacol. Ther. 2013, 29, 855-864; and Ligtenberg, et al., Mol. Therapy, 2018, 26, 1482-1493, the disclosures of which are incorporated by reference herein. In an embodiment, delivery of sdRNA to a TIL population is accomplished without use of electroporation, SQZ, or other methods, instead using a 1 to 3 day period in which a TIL population is exposed to sdRNA at a concentration of 1 μM/10,000 PBLs in medium. In certain embodiments, the method comprises delivery sdRNA to a PBLs population comprising exposing the PBLs population to sdRNA at a concentration of 1 μM/10,000 PBLs in medium for a period of between 1 to 3 days. In an embodiment, delivery of sdRNA to a TIL population is accomplished using a 1 to 3 day period in which a TIL population is exposed to sdRNA at a concentration of 10 μM/10,000 PBLs in medium. In an embodiment, delivery of sdRNA to a TIL population is accomplished using a 1 to 3 day period in which a TIL population is exposed to sdRNA at a concentration of 50 μM/10,000 PBLs in medium. In an embodiment, delivery of sdRNA to a TIL population is accomplished using a 1 to 3 day period in which a TIL population is exposed to sdRNA at a concentration of between 0.1 μM/10,000 PBLs and 50 μM/10,000 PBLs in medium. In an embodiment, delivery of sdRNA to a TIL population is accomplished using a 1 to 3 day period in which a TIL population is exposed to sdRNA at a concentration of between 0.1 μM/10,000 PBLs and 50 μM/10,000 PBLs in medium, wherein the exposure to sdRNA is performed two, three, four, or five times by addition of fresh sdRNA to the media. Other suitable processes are described, for example, in U.S. Patent Application Publication No. US 2011/0039914 A1, US 2013/0131141 A1, and US 2013/0131142 A1, and U.S. Pat. No. 9,080,171, the disclosures of which are incorporated by reference herein.

In some embodiments, sdRNA is inserted into a population of PBLs during manufacturing. In some embodiments, the sdRNA encodes RNA that interferes with NOTCH 1/2 ICD, PD-1, CTLA-4 TIM-3, LAG-3, TIGIT, TGFβ, TGFBR2, cAMP protein kinase A (PKA), BAFF BR3, CISH, and/or CBLB. In some embodiments, the reduction in expression is determined based on a percentage of gene silencing, for example, as assessed by flow cytometry and/or qPCR. In some embodiments, there is a reduction in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is a reduction in expression of at least about 65%, about 70%, about 75%, about 80%, about 85%, about 90%, or about 95%. In some embodiments, there is a reduction in expression of at least about 75%, about 80%, about 85%, about 90%, or about 95%. In some embodiments, there is a reduction in expression of at least about 80%, about 85%, about 90%, or about 95%. In some embodiments, there is a reduction in expression of at least about 85%, about 90%, or about 95%. In some embodiments, there is a reduction in expression of at least about 80%. In some embodiments, there is a reduction in expression of at least about 85%. In some embodiments, there is a reduction in expression of at least about 90%. In some embodiments, there is a reduction in expression of at least about 95%. In some embodiments, there is a reduction in expression of at least about 99%.

The self-deliverable RNAi technology based on the chemical modification of siRNAs can be employed with the methods of the present invention to successfully deliver the sdRNAs to the PBLs as described herein. The combination of backbone modifications with asymmetric siRNA structure and a hydrophobic ligand (see, for example, Ligtenberg, et al., Mol. Therapy, 2018 and US20160304873) allow sdRNAs to penetrate cultured mammalian cells without additional formulations and methods by simple addition to the culture media, capitalizing on the nuclease stability of sdRNAs. This stability allows the support of constant levels of RNAi-mediated reduction of target gene activity simply by maintaining the active concentration of sdRNA in the media. While not being bound by theory, the backbone stabilization of sdRNA provides for extended reduction in gene expression effects which can last for months in non-dividing cells.

In some embodiments, over 95% transfection efficiency of PBLs and a reduction in expression of the target by various specific sdRNA occurs. In some embodiments, sdRNAs containing several unmodified ribose residues were replaced with fully modified sequences to increase potency and/or the longevity of RNAi effect. In some embodiments, a reduction in expression effect is maintained for 12 hours, 24 hours, 36 hours, 48 hours, 5 days, 6 days, 7 days, or 8 days or more. In some embodiments, the reduction in expression effect decreases at 10 days or more post sdRNA treatment of the PBLs. In some embodiments, more than 70% reduction in expression of the target expression is maintained. In some embodiments, more than 70% reduction in expression of the target expression is maintained in PBLs. In some embodiments, a reduction in expression in the PD-1/PD-L1 pathway allows for the PBLs to exhibit a more potent in vivo effect, which is in some embodiments, due to the avoidance of the suppressive effects of the PD-1/PD-L1 pathway. In some embodiments, a reduction in expression of PD-1 by sdRNA results in an increase TIL proliferation.

Small interfering RNA (siRNA), sometimes known as short interfering RNA or silencing RNA, is a double stranded RNA molecule, generally 19-25 base pairs in length. siRNA is used in RNA interference (RNAi), where it interferes with expression of specific genes with complementary nucleotide sequences.

Double stranded DNA (dsRNA) can be generally used to define any molecule comprising a pair of complementary strands of RNA, generally a sense (passenger) and antisense (guide) strands, and may include single-stranded overhang regions. The term dsRNA, contrasted with siRNA, generally refers to a precursor molecule that includes the sequence of an siRNA molecule which is released from the larger dsRNA molecule by the action of cleavage enzyme systems, including Dicer.

sdRNA (self-deliverable RNA) are a new class of covalently modified RNAi compounds that do not require a delivery vehicle to enter cells and have improved pharmacology compared to traditional siRNAs. “Self-deliverable RNA” or “sdRNA” is a hydrophobically modified RNA interfering-antisense hybrid, demonstrated to be highly efficacious in vitro in primary cells and in vivo upon local administration. Robust uptake and/or silencing without toxicity has been demonstrated. sdRNAs are generally asymmetric chemically modified nucleic acid molecules with minimal double stranded regions. sdRNA molecules typically contain single stranded regions and double stranded regions, and can contain a variety of chemical modifications within both the single stranded and double stranded regions of the molecule. Additionally, the sdRNA molecules can be attached to a hydrophobic conjugate such as a conventional and advanced sterol-type molecule, as described herein. sdRNAs and associated methods for making such sdRNAs have also been described extensively in, for example, US20160304873, WO2010033246, WO2017070151, WO2009102427, WO2011119887, WO2010033247A2, WO2009045457, WO2011119852, all of which are incorporated by reference herein in their entireties for all purposes. To optimize sdRNA structure, chemistry, targeting position, sequence preferences, and the like, a proprietary algorithm has been developed and utilized for sdRNA potency prediction (see, for example, US 20160304873). Based on these analyses, functional sdRNA sequences have been generally defined as having over 70% reduction in expression at 1 μM concentration, with a probability over 40%.

In some embodiments, the sdRNA sequences used in the invention exhibit a 70% reduction in expression of the target gene. In some embodiments, the sdRNA sequences used in the invention exhibit a 75% reduction in expression of the target gene.

In some embodiments, the sdRNA sequences used in the invention exhibit an 80% reduction in expression of the target gene. In some embodiments, the sdRNA sequences used in the invention exhibit an 85% reduction in expression of the target gene. In some embodiments, the sdRNA sequences used in the invention exhibit a 90% reduction in expression of the target gene. In some embodiments, the sdRNA sequences used in the invention exhibit a 95% reduction in expression of the target gene. In some embodiments, the sdRNA sequences used in the invention exhibit a 99% reduction in expression of the target gene. In some embodiments, the sdRNA sequences used in the invention exhibit a reduction in expression of the target gene when delivered at a concentration of about 0.25 μM to about 4 μM. In some embodiments, the sdRNA sequences used in the invention exhibit a reduction in expression of the target gene when delivered at a concentration of about 0.25 μM. In some embodiments, the sdRNA sequences used in the invention exhibit a reduction in expression of the target gene when delivered at a concentration of about 0.5 μM. In some embodiments, the sdRNA sequences used in the invention exhibit a reduction in expression of the target gene when delivered at a concentration of about 0.75 μM. In some embodiments, the sdRNA sequences used in the invention exhibit a reduction in expression of the target gene when delivered at a concentration of about 1.0 μM. In some embodiments, the sdRNA sequences used in the invention exhibit a reduction in expression of the target gene when delivered at a concentration of about 1.25 μM. In some embodiments, the sdRNA sequences used in the invention exhibit a reduction in expression of the target gene when delivered at a concentration of about 1.5 μM. In some embodiments, the sdRNA sequences used in the invention exhibit a reduction in expression of the target gene when delivered at a concentration of about 1.75 μM. In some embodiments, the sdRNA sequences used in the invention exhibit a reduction in expression of the target gene when delivered at a concentration of about 2.0 μM. In some embodiments, the sdRNA sequences used in the invention exhibit a reduction in expression of the target gene when delivered at a concentration of about 2.25 μM. In some embodiments, the sdRNA sequences used in the invention exhibit a reduction in expression of the target gene when delivered at a concentration of about 2.5 μM. In some embodiments, the sdRNA sequences used in the invention exhibit a reduction in expression of the target gene when delivered at a concentration of about 2.75 μM. In some embodiments, the sdRNA sequences used in the invention exhibit a reduction in expression of the target gene when delivered at a concentration of about 3.0 μM. In some embodiments, the sdRNA sequences used in the invention exhibit a reduction in expression of the target gene when delivered at a concentration of about 3.25 μM. In some embodiments, the sdRNA sequences used in the invention exhibit a reduction in expression of the target gene when delivered at a concentration of about 3.5 μM. In some embodiments, the sdRNA sequences used in the invention exhibit a reduction in expression of the target gene when delivered at a concentration of about 3.75 μM. In some embodiments, the sdRNA sequences used in the invention exhibit a reduction in expression of the target gene when delivered at a concentration of about 4.0 μM.

In some embodiments, the oligonucleotide agents comprise one or more modification to increase stability and/or effectiveness of the therapeutic agent, and to effect efficient delivery of the oligonucleotide to the cells or tissue to be treated. Such modifications can include a 2′-O-methyl modification, a 2′-O-Fluro modification, a diphosphorothioate modification, 2′ F modified nucleotide, a2′-O-methyl modified and/or a 2′deoxy nucleotide. In some embodiments, the oligonucleotide is modified to include one or more hydrophobic modifications including, for example, sterol, cholesterol, vitamin D, naphtyl, isobutyl, benzyl, indol, tryptophane, and/or phenyl. In an additional particular embodiment, chemically modified nucleotides are combination of phosphorothioates, 2′-O-methyl, 2′deoxy, hydrophobic modifications and phosphorothioates. In some embodiments, the sugars can be modified and modified sugars can include but are not limited to D-ribose, 2′-O-alkyl (including 2′-O-methyl and 2′-O-ethyl), i.e., 2′-alkoxy, 2′-amino, 2′-S-alkyl, 2′-halo (including 2′-fluoro), T-methoxyethoxy, 2′-allyloxy (—OCH₂CH═CH₂), 2′-propargyl, 2′-propyl, ethynyl, ethenyl, propenyl, and cyano and the like. In one embodiment, the sugar moiety can be a hexose and incorporated into an oligonucleotide as described (Augustyns, K., et al., Nucl. Acids. Res. 18:4711 (1992)).

In some embodiments, the double-stranded oligonucleotide of the invention is double-stranded over its entire length, i.e., with no overhanging single-stranded sequence at either end of the molecule, i.e., is blunt-ended. In some embodiments, the individual nucleic acid molecules can be of different lengths. In other words, a double-stranded oligonucleotide of the invention is not double-stranded over its entire length. For instance, when two separate nucleic acid molecules are used, one of the molecules, e.g., the first molecule comprising an antisense sequence, can be longer than the second molecule hybridizing thereto (leaving a portion of the molecule single-stranded). In some embodiments, when a single nucleic acid molecule is used a portion of the molecule at either end can remain single-stranded.

In some embodiments, a double-stranded oligonucleotide of the invention contains mismatches and/or loops or bulges, but is double-stranded over at least about 70% of the length of the oligonucleotide. In some embodiments, a double-stranded oligonucleotide of the invention is double-stranded over at least about 80% of the length of the oligonucleotide. In another embodiment, a double-stranded oligonucleotide of the invention is double-stranded over at least about 90%-95% of the length of the oligonucleotide. In some embodiments, a double-stranded oligonucleotide of the invention is double-stranded over at least about 96%-98% of the length of the oligonucleotide. In some embodiments, the double-stranded oligonucleotide of the invention contains at least or up to 1, 2, 3, 4, 5, 6, 7, 8, 9, 10, 11, 12, 13, 14, or 15 mismatches.

In some embodiments, the oligonucleotide can be substantially protected from nucleases e.g., by modifying the 3′ or 5′ linkages (e.g., U.S. Pat. No. 5,849,902 and WO 98/13526). For example, oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃^{2″}), hydrogen phosphonate, or phosphoramidite). “Blocking groups” can also include “end blocking groups” or “exonuclease blocking groups” which protect the 5′ and 3′ termini of the oligonucleotide, including modified nucleotides and non-nucleotide exonuclease resistant structures.

In some embodiments, at least a portion of the contiguous polynucleotides within the sdRNA are linked by a substitute linkage, e.g., a phosphorothioate linkage.

In some embodiments, chemical modification can lead to at least a 1.5, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 425, 450, 475, 500 enhancements in cellular uptake. In some embodiments, at least one of the C or U residues includes a hydrophobic modification. In some embodiments, a plurality of Cs and Us contain a hydrophobic modification. In some embodiments, at least 10%, 15%, 20%, 30%, 40%, 50%, 55%, 60% 65%, 70%, 75%, 80%, 85%, 90% or at least 95% of the Cs and Us can contain a hydrophobic modification. In some embodiments, all of the Cs and Us contain a hydrophobic modification.

In some embodiments, the sdRNA or sd-rxRNAs exhibit enhanced endosomal release of sd-rxRNA molecules through the incorporation of protonatable amines. In some embodiments, protonatable amines are incorporated in the sense strand (in the part of the molecule which is discarded after RISC loading). In some embodiments, the sdRNA compounds of the invention comprise an asymmetric compound comprising a duplex region (required for efficient RISC entry of 10-15 bases long) and single stranded region of 4-12 nucleotides long; with a 13 nucleotide duplex. In some embodiments, a 6 nucleotide single stranded region is employed. In some embodiments, the single stranded region of the sdRNA comprises 2-12 phosphorothioate internucleotide linkages (referred to as phosphorothioate modifications). In some embodiments, 6-8 phosphorothioate internucleotide linkages are employed. In some embodiments, the sdRNA compounds of the invention also include a unique chemical modification pattern, which provides stability and is compatible with RISC entry.

The guide strand, for example, may also be modified by any chemical modification which confirms stability without interfering with RISC entry. In some embodiments, the chemical modification pattern in the guide strand includes the majority of C and U nucleotides being 2′ F modified and the 5′ end being phosphorylated.

In some embodiments, at least 30% of the nucleotides in the sdRNA or sd-rxRNA are modified. In some embodiments, at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the nucleotides in the sdRNA or sd-rxRNA are modified. In some embodiments, 100% of the nucleotides in the sdRNA or sd-rxRNA are modified.

In some embodiments, the sdRNA molecules have minimal double stranded regions. In some embodiments the region of the molecule that is double stranded ranges from 8-15 nucleotides long. In some embodiments, the region of the molecule that is double stranded is 8, 9, 10, 11, 12, 13, 14 or 15 nucleotides long. In some embodiments the double stranded region is 13 nucleotides long. There can be 100% complementarity between the guide and passenger strands, or there may be one or more mismatches between the guide and passenger strands. In some embodiments, on one end of the double stranded molecule, the molecule is either blunt-ended or has a one-nucleotide overhang. The single stranded region of the molecule is in some embodiments between 4-12 nucleotides long. In some embodiments, the single stranded region can be 4, 5, 6, 7, 8, 9, 10, 11 or 12 nucleotides long. In some embodiments, the single stranded region can also be less than 4 or greater than 12 nucleotides long. In certain embodiments, the single stranded region is 6 or 7 nucleotides long.

In some embodiments, the sdRNA molecules have increased stability. In some instances, a chemically modified sdRNA or sd-rxRNA molecule has a half-life in media that is longer than 1, 2, 3, 4, 5, 6, 7, 8, 9, 10, 11, 12, 13, 14, 15, 16, 17, 18, 19, 20, 21, 22, 23, 24 or more than 24 hours, including any intermediate values. In some embodiments, the sd-rxRNA has a half-life in media that is longer than 12 hours.

In some embodiments, the sdRNA is optimized for increased potency and/or reduced toxicity. In some embodiments, nucleotide length of the guide and/or passenger strand, and/or the number of phosphorothioate modifications in the guide and/or passenger strand, can in some aspects influence potency of the RNA molecule, while replacing 2′-fluoro (2′F) modifications with 2′-O-methyl (2′OMe) modifications can in some aspects influence toxicity of the molecule. In some embodiments, reduction in 2′F content of a molecule is predicted to reduce toxicity of the molecule. In some embodiments, the number of phosphorothioate modifications in an RNA molecule can influence the uptake of the molecule into a cell, for example the efficiency of passive uptake of the molecule into a cell. In some embodiments, the sdRNA has no 2′F modification and yet are characterized by equal efficacy in cellular uptake and tissue penetration.

In some embodiments, a guide strand is approximately 18-19 nucleotides in length and has approximately 2-14 phosphate modifications. For example, a guide strand can contain 2, 3, 4, 5, 6, 7, 8, 9, 10, 11, 12, 13, 14 or more than 14 nucleotides that are phosphate-modified. The guide strand may contain one or more modifications that confer increased stability without interfering with RISC entry. The phosphate modified nucleotides, such as phosphorothioate modified nucleotides, can be at the 3′ end, 5′ end or spread throughout the guide strand. In some embodiments, the 3′ terminal 10 nucleotides of the guide strand contain 1, 2, 3, 4, 5, 6, 7, 8, 9 or 10 phosphorothioate modified nucleotides. The guide strand can also contain 2′F and/or 2′OMe modifications, which can be located throughout the molecule. In some embodiments, the nucleotide in position one of the guide strand (the nucleotide in the most 5′ position of the guide strand) is 2′OMe modified and/or phosphorylated. C and U nucleotides within the guide strand can be 2′F modified. For example, C and U nucleotides in positions 2-10 of a 19 nt guide strand (or corresponding positions in a guide strand of a different length) can be 2′F modified. C and U nucleotides within the guide strand can also be 2′OMe modified. For example, C and U nucleotides in positions 11-18 of a 19 nt guide strand (or corresponding positions in a guide strand of a different length) can be 2′OMe modified. In some embodiments, the nucleotide at the most 3′ end of the guide strand is unmodified. In certain embodiments, the majority of Cs and Us within the guide strand are 2′F modified and the 5′ end of the guide strand is phosphorylated. In other embodiments, position 1 and the Cs or Us in positions 11-18 are 2′OMe modified and the 5′ end of the guide strand is phosphorylated. In other embodiments, position 1 and the Cs or Us in positions 11-18 are 2′OMe modified, the 5′ end of the guide strand is phosphorylated, and the Cs or Us in position 2-10 are 2′F modified.

The self-deliverable RNAi technology provides a method of directly transfecting cells with the RNAi agent, without the need for additional formulations or techniques. The ability to transfect hard-to-transfect cell lines, high in vivo activity, and simplicity of use, are characteristics of the compositions and methods that present significant functional advantages over traditional siRNA-based techniques, and as such, the sdRNA methods are employed in several embodiments related to the methods of reduction in expression of the target gene in the PBLs of the present invention. The sdRNAi methods allow direct delivery of chemically synthesized compounds to a wide range of primary cells and tissues, both ex-vivo and in vivo. The sdRNAs described in some embodiments of the invention herein are commercially available from Advirna LLC, Worcester, Mass., USA.

The sdRNA are formed as hydrophobically-modified siRNA-antisense oligonucleotide hybrid structures, and are disclosed, for example in Byrne et al., December 2013, J. Ocular Pharmacology and Therapeutics, 29(10): 855-864, incorporated by reference herein in its entirety.

In some embodiments, the sdRNA oligonucleotides can be delivered to the PBLs described herein using sterile electroporation. In certain embodiments, the method comprises sterile electroporation of a population of PBLs to deliver sdRNA oligonucleotides.

In some embodiments, the oligonucleotides can be delivered to the cells in combination with a transmembrane delivery system. In some embodiments, this transmembrane delivery system comprises lipids, viral vectors, and the like. In some embodiments, the oligonucleotide agent is a self-delivery RNAi agent, that does not require any delivery agents. In certain embodiments, the method comprises use of a transmembrane delivery system to deliver sdRNA oligonucleotides to a population of PBLs.

Oligonucleotides and oligonucleotide compositions are contacted with (e.g., brought into contact with, also referred to herein as administered or delivered to) and taken up by PBLs described herein, including through passive uptake by PBLs. The sdRNA can be added to the PBLs as described herein during the step of culturing in the first culture medium, after the step of culturing in the first culture medium, before or during the step of culturing in the second culture medium, before the harvest step, during or after harvest step, before or during the step of final formulation and/or transfer to infusion bag, as well as before any optional cryopreservation step. Moreover, sdRNA can be added after thawing from any cryopreservation step. In an embodiment, one or more sdRNAs targeting genes as described herein, including PD-1, LAG-3, TIM-3, CISH, and CBLB, may be added to cell culture media comprising PBLs and other agents at concentrations selected from the group consisting of 100 nM to 20 mM, 200 nM to 10 mM, 500 nm to 1 mM, 1 μM to 100 μM, and 1 μM to 100 μM. In an embodiment, one or more sdRNAs targeting genes as described herein, including PD-1, LAG-3, TIM-3, CISH, and CBLB, may be added to cell culture media comprising PBLs and other agents at amounts selected from the group consisting of 0.1 μM sdRNA/10,000 PBLs/100 μL media, 0.5 μM sdRNA/10,000 PBLs/100 μL media, 0.75 μM sdRNA/10,000 PBLs/100 μL media, 1 μM sdRNA/10,000 PBLs/100 μL media, 1.25 μM sdRNA/10,000 PBLs/100 μL media, 1.5 μM sdRNA/10,000 PBLs/100 μL media, 2 μM sdRNA/10,000 PBLs/100 μL media, 5 μM sdRNA/10,000 PBLs/100 μL media, or 10 μM sdRNA/10,000 PBLs/100 μL media. In an embodiment, one or more sdRNAs targeting genes as described herein, including PD-1, LAG-3, TIM-3, CISH, and CBLB, may be added to TIL cultures during the culturing steps twice a day, once a day, every two days, every three days, every four days, every five days, every six days, or every seven days.

Oligonucleotide compositions of the invention, including sdRNA, can be contacted with PBLs as described herein during the expansion process, for example by dissolving sdRNA at high concentrations in cell culture media and allowing sufficient time for passive uptake to occur. In certain embodiments, the method of the present invention comprises contacting a population of PBLs with an oligonucleotide composition as described herein. In certain embodiments, the method comprises dissolving an oligonucleotide e.g. sdRNA in a cell culture media and contacting the cell culture media with a population of PBLs. The PBLs may be a first population, a second population and/or a third population as described herein.

In some embodiments, delivery of oligonucleotides into cells can be enhanced by suitable art recognized methods including calcium phosphate, DMSO, glycerol or dextran, electroporation, or by transfection, e.g., using cationic, anionic, or neutral lipid compositions or liposomes using methods known in the art (see, e.g., WO 90/14074; WO 91/16024; WO 91/17424; U.S. Pat. No. 4,897,355; Bergan et a 1993. Nucleic Acids Research. 21:3567).

In some embodiments, more than one sdRNA is used to reduce expression of a target gene. In some embodiments, one or more of PD-1, TIM-3, CBLB, LAG3 and/or CISH targeting sdRNAs are used together. In some embodiments, a PD-1 sdRNA is used with one or more of TIM-3, CBLB, LAG3 and/or CISH in order to reduce expression of more than one gene target. In some embodiments, a LAG3 sdRNA is used in combination with a CISH targeting sdRNA to reduce gene expression of both targets. In some embodiments, the sdRNAs targeting one or more of PD-1, TIM-3, CBLB, LAG3 and/or CISH herein are commercially available from Advirna LLC, Worcester, Mass., USA.

In some embodiments, the sdRNA targets a gene selected from the group consisting of PD-1, LAG3, TIM3, CTLA-4, TIGIT, CISH, TGFβR2, PKA, CBLB, BAFF (BR3), and combinations thereof. In some embodiments, the sdRNA targets a gene selected from the group consisting of PD-1, LAG3, TIM3, CTLA-4, TIGIT, CISH, TGFβR2, PKA, CBLB, BAFF (BR3), and combinations thereof. In some embodiments, one sdRNA targets PD-1 and another sdRNA targets a gene selected from the group consisting of LAG3, TIM3, CTLA-4, TIGIT, CISH, TGFβR2, PKA, CBLB, BAFF (BR3), and combinations thereof. In some embodiments, the sdRNA targets a gene selected from PD-1, LAG-3, CISH, CBLB, TIM3, and combinations thereof. In some embodiments, the sdRNA targets a gene selected from PD-1 and one of LAG3, CISH, CBLB, TIM3, and combinations thereof. In some embodiments, one sdRNA targets PD-1 and one sdRNA targets LAG3. In some embodiments, one sdRNA targets PD-1 and one sdRNA targets CISH. In some embodiments, one sdRNA targets PD-1 and one sdRNA targets CBLB. In some embodiments, one sdRNA targets LAG3 and one sdRNA targets CISH. In some embodiments, one sdRNA targets LAG3 and one sdRNA targets CBLB. In some embodiments, one sdRNA targets CISH and one sdRNA targets CBLB. In some embodiments, one sdRNA targets TIM3 and one sdRNA targets PD-1. In some embodiments, one sdRNA targets TIM3 and one sdRNA targets LAG3. In some embodiments, one sdRNA targets TIM3 and one sdRNA targets CISH. In some embodiments, one sdRNA targets TIM3 and one sdRNA targets CBLB.

As discussed above, embodiments of the present invention provide PBLs that have been genetically modified via gene-editing to enhance their therapeutic effect. Embodiments of the present invention embrace genetic editing through nucleotide insertion (RNA or DNA) into a population of PBLs for both promotion of the expression of one or more proteins and inhibition of the expression of one or more proteins, as well as combinations thereof. Embodiments of the present invention also provide methods for expanding PBLs into a therapeutic population, wherein the methods comprise gene-editing the PBLs. There are several gene-editing technologies that may be used to genetically modify a population of PBLs, which are suitable for use in accordance with the present invention.

In some embodiments, the method comprises a method of genetically modifying a population of PBLs which include the step of stable incorporation of genes for production of one or more proteins. In an embodiment, a method of genetically modifying a population of PBLs includes the step of retroviral transduction. In an embodiment, a method of genetically modifying a population of PBLs includes the step of lentiviral transduction. Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein. In an embodiment, a method of genetically modifying a population of PBLs includes the step of gamma-retroviral transduction. Gamma-retroviral transduction systems are known in the art and are described, e.g., Cepko and Pear, Cur. Prot. Mol. Biol. 1996, 9.9.1-9.9.16, the disclosure of which is incorporated by reference herein. In an embodiment, a method of genetically modifying a population of PBLs includes the step of transposon-mediated gene transfer. Transposon-mediated gene transfer systems are known in the art and include systems wherein the transposase is provided as DNA expression vector or as an expressible RNA or a protein such that long-term expression of the transposase does not occur in the transgenic cells, for example, a transposase provided as an mRNA (e.g., an mRNA comprising a cap and poly-A tail). Suitable transposon-mediated gene transfer systems, including the salmonid-type Tel-like transposase (SB or Sleeping Beauty transposase), such as SB10, SB11, and SB100x, and engineered enzymes with increased enzymatic activity, are described in, e.g., Hackett, et al., Mol. Therapy 2010, 18, 674-83 and U.S. Pat. No. 6,489,458, the disclosures of each of which are incorporated by reference herein.

In an embodiment, the method comprises a method of genetically modifying a population of PBLs e.g. a first population, a second population and/or a third population as described herein. In an embodiment, a method of genetically modifying a population of PBLs includes the step of stable incorporation of genes for production or inhibition (e.g., silencing) of one or more proteins. In an embodiment, a method of genetically modifying a population of PBLs includes the step of electroporation. Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. Other electroporation methods known in the art, such as those described in U.S. Pat. Nos. 5,019,034; 5,128,257; 5,137,817; 5,173,158; 5,232,856; 5,273,525; 5,304,120; 5,318,514; 6,010,613 and 6,078,490, the disclosures of which are incorporated by reference herein, may be used. In an embodiment, the electroporation method is a sterile electroporation method. In an embodiment, the electroporation method is a pulsed electroporation method. In an embodiment, the electroporation method is a pulsed electroporation method comprising the steps of treating PBLs with pulsed electrical fields to alter, manipulate, or cause defined and controlled, permanent or temporary changes in the PBLs, comprising the step of applying a sequence of at least three single, operator-controlled, independently programmed, DC electrical pulses, having field strengths equal to or greater than 100 V/cm, to the PBLs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses. In an embodiment, the electroporation method is a pulsed electroporation method comprising the steps of treating PBLs with pulsed electrical fields to alter, manipulate, or cause defined and controlled, permanent or temporary changes in the PBLs, comprising the step of applying a sequence of at least three single, operator-controlled, independently programmed, DC electrical pulses, having field strengths equal to or greater than 100 V/cm, to the PBLs, wherein at least two of the at least three pulses differ from each other in pulse amplitude. In an embodiment, the electroporation method is a pulsed electroporation method comprising the steps of treating PBLs with pulsed electrical fields to alter, manipulate, or cause defined and controlled, permanent or temporary changes in the PBLs, comprising the step of applying a sequence of at least three single, operator-controlled, independently programmed, DC electrical pulses, having field strengths equal to or greater than 100 V/cm, to the PBLs, wherein at least two of the at least three pulses differ from each other in pulse width. In an embodiment, the electroporation method is a pulsed electroporation method comprising the steps of treating PBLs with pulsed electrical fields to alter, manipulate, or cause defined and controlled, permanent or temporary changes in the PBLs, comprising the step of applying a sequence of at least three single, operator-controlled, independently programmed, DC electrical pulses, having field strengths equal to or greater than 100 V/cm, to the PBLs, wherein a first pulse interval for a first set of two of the at least three pulses is different from a second pulse interval for a second set of two of the at least three pulses. In an embodiment, the electroporation method is a pulsed electroporation method comprising the steps of treating PBLs with pulsed electrical fields to induce pore formation in the PBLs, comprising the step of applying a sequence of at least three DC electrical pulses, having field strengths equal to or greater than 100 V/cm, to PBLs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses, such that induced pores are sustained for a relatively long period of time, and such that viability of the PBLs is maintained. In an embodiment, a method of genetically modifying a population of PBLs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In an embodiment, a method of genetically modifying a population of PBLs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In an embodiment, a method of genetically modifying a population of PBLs includes the step of transfection using methods described in U.S. Pat. Nos. 5,766,902; 6,025,337; 6,410,517; 6,475,994; and 7,189,705; the disclosures of each of which are incorporated by reference herein. The PBLs may be a first population, a second population and/or a third population of PBLs as described herein.

According to an embodiment, the gene-editing process may comprise the use of a programmable nuclease that mediates the generation of a double-strand or single-strand break at one or more immune checkpoint genes. Such programmable nucleases enable precise genome editing by introducing breaks at specific genomic loci, i.e., they rely on the recognition of a specific DNA sequence within the genome to target a nuclease domain to this location and mediate the generation of a double-strand break at the target sequence. A double-strand break in the DNA subsequently recruits endogenous repair machinery to the break site to mediate genome editing by either non-homologous end-joining (NHEJ) or homology-directed repair (HDR). Thus, the repair of the break can result in the introduction of insertion/deletion mutations that disrupt (e.g., silence, repress, or enhance) the target gene product.

Major classes of nucleases that have been developed to enable site-specific genomic editing include zinc finger nucleases (ZFNs), transcription activator-like nucleases (TALENs), and CRISPR-associated nucleases (e.g., CRISPR/Cas9). These nuclease systems can be broadly classified into two categories based on their mode of DNA recognition: ZFNs and TALENs achieve specific DNA binding via protein-DNA interactions, whereas CRISPR systems, such as Cas9, are targeted to specific DNA sequences by a short RNA guide molecule that base-pairs directly with the target DNA and by protein-DNA interactions. See, e.g., Cox et al., Nature Medicine, 2015, Vol. 21, No. 2.

Non-limiting examples of gene-editing methods that may be used in accordance with TIL expansion methods of the present invention include CRISPR methods, TALE methods, and ZFN methods, which are described in more detail below. According to an embodiment, a method for expanding PBLs into a therapeutic population may be carried out in accordance with any embodiment of the methods described herein (e.g., GEN 3 process) or as described in PCT/US2017/058610, PCT/US2018/012605, or PCT/US2018/012633, wherein the method further comprises gene-editing at least a portion of the PBLs by one or more of a CRISPR method, a TALE method or a ZFN method, in order to generate PBLs that can provide an enhanced therapeutic effect. According to an embodiment, gene-edited PBLs can be evaluated for an improved therapeutic effect by comparing them to non-modified PBLs in vitro, e.g., by evaluating in vitro effector function, cytokine profiles, etc. compared to unmodified PBLs. In certain embodiments, the method comprises gene editing a population of PBLs using CRISPR, TALE and/or ZFN methods.

In some embodiments of the present invention, electroporation is used for delivery of a gene editing system, such as CRISPR, TALEN, and ZFN systems. In some embodiments of the present invention, the electroporation system is a flow electroporation system. An example of a suitable flow electroporation system suitable for use with some embodiments of the present invention is the commercially-available MaxCyte STX system. There are several alternative commercially-available electroporation instruments which may be suitable for use with the present invention, such as the AgilePulse system or ECM 830 available from BTX-Harvard Apparatus, Cellaxess Elektra (Cellectricon), Nucleofector (Lonza/Amaxa), GenePulser MXcell (BIORAD), iPorator-96 (Primax) or siPORTer96 (Ambion). In some embodiments of the present invention, the electroporation system forms a closed, sterile system with the remainder of the TIL expansion method. In some embodiments of the present invention, the electroporation system is a pulsed electroporation system as described herein, and forms a closed, sterile system with the remainder of the TIL expansion method.

A method for expanding PBLs into a therapeutic population may be carried out in accordance with any embodiment of the methods described herein (e.g., process GEN 3) or as described in PCT/US2017/058610, PCT/US2018/012605, or PCT/US2018/012633, wherein the method further comprises gene-editing at least a portion of the PBLs by a CRISPR method (e.g., CRISPR/Cas9 or CRISPR/Cpf1). According to particular embodiments, the use of a CRISPR method during the TIL expansion process causes expression of one or more immune checkpoint genes to be silenced or reduced in at least a portion of the therapeutic population of PBLs. Alternatively, the use of a CRISPR method during the TIL expansion process causes expression of one or more immune checkpoint genes to be enhanced in at least a portion of the therapeutic population of PBLs.

CRISPR stands for “Clustered Regularly Interspaced Short Palindromic Repeats.” A method of using a CRISPR system for gene editing is also referred to herein as a CRISPR method. There are three types of CRISPR systems which incorporate RNAs and Cas proteins, and which may be used in accordance with the present invention: Types I, II, and III. The Type II CRISPR (exemplified by Cas9) is one of the most well-characterized systems.

CRISPR technology was adapted from the natural defense mechanisms of bacteria and archaea (the domain of single-celled microorganisms). These organisms use CRISPR-derived RNA and various Cas proteins, including Cas9, to foil attacks by viruses and other foreign bodies by chopping up and destroying the DNA of a foreign invader. A CRISPR is a specialized region of DNA with two distinct characteristics: the presence of nucleotide repeats and spacers. Repeated sequences of nucleotides are distributed throughout a CRISPR region with short segments of foreign DNA (spacers) interspersed among the repeated sequences. In the type II CRISPR/Cas system, spacers are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Target recognition by the Cas9 protein requires a “seed” sequence within the crRNA and a conserved dinucleotide-containing protospacer adjacent motif (PAM) sequence upstream of the crRNA-binding region. The CRISPR/Cas system can thereby be retargeted to cleave virtually any DNA sequence by redesigning the crRNA. The crRNA and tracrRNA in the native system can be simplified into a single guide RNA (sgRNA) of approximately 100 nucleotides for use in genetic engineering. The CRISPR/Cas system is directly portable to human cells by co-delivery of plasmids expressing the Cas9 endo-nuclease and the necessary crRNA components. Different variants of Cas proteins may be used to reduce targeting limitations (e.g., orthologs of Cas9, such as Cpf1).

Non-limiting examples of genes that may be silenced or inhibited by permanently gene-editing PBLs via a CRISPR method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, and GUCY1B3.

Non-limiting examples of genes that may be enhanced by permanently gene-editing PBLs via a CRISPR method include CCR2, CCR4, CCR5, CXCR2, CXCR3, CX3CR1, IL-2, IL12, IL-15, and IL-21.

Examples of systems, methods, and compositions for altering the expression of a target gene sequence by a CRISPR method, and which may be used in accordance with embodiments of the present invention, are described in U.S. Pat. Nos. 8,697,359; 8,993,233; 8,795,965; 8,771,945; 8,889,356; 8,865,406; 8,999,641; 8,945,839; 8,932,814; 8,871,445; 8,906,616; and 8,895,308, which are incorporated by reference herein. Resources for carrying out CRISPR methods, such as plasmids for expressing CRISPR/Cas9 and CRISPR/Cpf1, are commercially available from companies such as GenScript.

In an embodiment, genetic modifications of populations of PBLs, as described herein, may be performed using the CRISPR/Cpf1 system as described in U.S. Pat. No. 9,790,490, the disclosure of which is incorporated by reference herein.

A method for expanding PBLs into a therapeutic population may be carried out in accordance with any embodiment of the methods described herein (e.g., process 2A) or as described in PCT/US2017/058610, PCT/US2018/012605, or PCT/US2018/012633, wherein the method further comprises gene-editing at least a portion of the PBLs by a TALE method. According to particular embodiments, the use of a TALE method during the TIL expansion process causes expression of one or more immune checkpoint genes to be silenced or reduced in at least a portion of the therapeutic population of PBLs. Alternatively, the use of a TALE method during the TIL expansion process causes expression of one or more immune checkpoint genes to be enhanced in at least a portion of the therapeutic population of PBLs.

TALE stands for “Transcription Activator-Like Effector” proteins, which include TALENs (“Transcription Activator-Like Effector Nucleases”). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Custom-designed TALE arrays are also commercially available through Cellectis Bioresearch (Paris, France), Transposagen Biopharmaceuticals (Lexington, Ky., USA), and Life Technologies (Grand Island, N.Y., USA). TALE and TALEN methods suitable for use in the present invention are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein.

Non-limiting examples of genes that may be silenced or inhibited by permanently gene-editing PBLs via a TALE method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, and GUCY1B3.

Non-limiting examples of genes that may be enhanced by permanently gene-editing PBLs via a TALE method include CCR2, CCR4, CCR5, CXCR2, CXCR3, CX3CR1, IL-2, IL12, IL-15, and IL-21.

Examples of systems, methods, and compositions for altering the expression of a target gene sequence by a TALE method, and which may be used in accordance with embodiments of the present invention, are described in U.S. Pat. No. 8,586,526, which is incorporated by reference herein.

A method for expanding PBLs into a therapeutic population may be carried out in accordance with any embodiment of the methods described herein (e.g., process GEN 3) or as described in PCT/US2017/058610, PCT/US2018/012605, or PCT/US2018/012633, wherein the method further comprises gene-editing at least a portion of the PBLs by a zinc finger or zinc finger nuclease method. According to particular embodiments, the use of a zinc finger method during the TIL expansion process causes expression of one or more immune checkpoint genes to be silenced or reduced in at least a portion of the therapeutic population of PBLs. Alternatively, the use of a zinc finger method during the TIL expansion process causes expression of one or more immune checkpoint genes to be enhanced in at least a portion of the therapeutic population of PBLs.

An individual zinc finger contains approximately 30 amino acids in a conserved ββα configuration. Several amino acids on the surface of the α-helix typically contact 3 bp in the major groove of DNA, with varying levels of selectivity. Zinc fingers have two protein domains. The first domain is the DNA binding domain, which includes eukaryotic transcription factors and contain the zinc finger. The second domain is the nuclease domain, which includes the FokI restriction enzyme and is responsible for the catalytic cleavage of DNA.

The DNA-binding domains of individual ZFNs typically contain between three and six individual zinc finger repeats and can each recognize between 9 and 18 base pairs. If the zinc finger domains are specific for their intended target site then even a pair of 3-finger ZFNs that recognize a total of 18 base pairs can, in theory, target a single locus in a mammalian genome. One method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Alternatively, selection-based approaches, such as oligomerized pool engineering (OPEN) can be used to select for new zinc-finger arrays from randomized libraries that take into consideration context-dependent interactions between neighboring fingers. Engineered zinc fingers are available commercially; Sangamo Biosciences (Richmond, Calif., USA) has developed a propriety platform (CompoZr®) for zinc-finger construction in partnership with Sigma-Aldrich (St. Louis, Mo., USA).

Non-limiting examples of genes that may be silenced or inhibited by permanently gene-editing PBLs via a zinc finger method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, and GUCY1B3.

Non-limiting examples of genes that may be enhanced by permanently gene-editing PBLs via a zinc finger method include CCR2, CCR4, CCR5, CXCR2, CXCR3, CX3CR1, IL-2, IL12, IL-15, and IL-21.

Examples of systems, methods, and compositions for altering the expression of a target gene sequence by a zinc finger method, which may be used in accordance with embodiments of the present invention, are described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, which are incorporated by reference herein.

Other examples of systems, methods, and compositions for altering the expression of a target gene sequence by a zinc finger method, which may be used in accordance with embodiments of the present invention, are described in Beane, et al., Mol. Therapy, 2015, 23 1380-1390, the disclosure of which is incorporated by reference herein.

### Chimeric Antigen Receptors and Genetically-Modified T-Cell Receptors

In some embodiments, the PBLs are optionally genetically engineered to include additional functionalities, including, but not limited to, a high-affinity T cell receptor (TCR), e.g., a TCR targeted at a tumor-associated antigen such as MAGE-1, HER2, or NY-ESO-1, or a chimeric antigen receptor (CAR) which binds to a tumor-associated cell surface molecule (e.g., mesothelin) or lineage-restricted cell surface molecule (e.g., CD19). In certain embodiments, the method comprises genetically engineering a population of PBLs to include a high-affinity T cell receptor (TCR), e.g., a TCR targeted at a tumor-associated antigen such as MAGE-1, HER2, or NY-ESO-1, or a CAR which binds to a tumor-associated cell surface molecule (e.g., mesothelin) or lineage-restricted cell surface molecule (e.g., CD19). In certain embodiments, the method comprises genetically engineering a population of PBLs to include a CAR specific for CD19, CD20, CD19 and CD20 (bispecific), CD30, CD33, CD123, PSMA, mesothelin, CE7, HER2/neu BCMA, EGFRvIII, HER2/CMV, IL13Rα2, human C4 folate receptor-alpha (αFR), or GD2.

In some embodiments, the PBLs expanded according to the methods of the present invention are genetically modified to target antigens through expression of chimeric antigen receptors (CARs). In some embodiments, the PBLs of the present invention are transduced with an expression vector comprising a nucleic acid encoding a CAR comprising a single chain variable fragment antibody fused with at least one endodomain of a T-cell signaling molecule. In some embodiments, the transducing step takes place at any time during the expansion process. In some embodiments, the transducing step takes place after the expanded cells are harvested. In some embodiments, the PBLs expanded according to the methods of the present invention include a polynucleotide capable of expression of a CAR.

In one embodiment, the CARs or nucleotides encoding CARs are prepared and transduced according to the disclosure in U.S. Pat. Nos. 9,328,156; 8,399,645; 7,446,179; 6,410,319; 7,446,190, and U.S. Patent Application Publication Nos. US 2015/0038684; US 2015/0031624; US 2014/0301993 A1; US 2014/0271582 A1; US 2015/0051266 A1; US 2014/0322275 A1; and US 2014/0004132 A1, the discloses of each of which is incorporated by reference herein. In U.S. Pat. No. 9,328,156, CAR-T cells are prepared to treat patients with B-cell lymphomas, and particularly CLL, and the embodiments discussed therein are useful in the present invention. For example, a CAR-T cell expressing a CD19 antigen binding domain, a transmembrane domain, a 4-1BB costimulatory signaling region, and a CD3 zeta signaling domain is useful in the present invention. In an embodiment of the invention, the CAR comprises a target-specific binding element, or antibody binding domain, a transmembrane domain, and a cytoplasmic domain. Hematopoietic tumor antigens (for the antibody binding domain) are well known in the art and include, for example, CD19, CD20, CD22, ROR1, Mesothelin, CD33/IL3Ra, c-Met, PSMA, Glycolipid F77, EGFRvIII, GD-2, NY-ESO-1 TCR, MAGE A3 TCR, and the like. In an embodiment, the transmembrane domain comprises the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, or CD154, and may be synthetic. In an embodiment, the cytoplasmic or signaling domain comprises a portion or all of the TCR zeta, FcR gamma, FcR beta, CD3 gamma, CD3 delta, CD3 epsilon, CD3 zeta, CD5, CD22, CD79a, CD79b, or CD66d domains. In an embodiment of the invention, the cytoplasmic or signaling domain may also include a co-stimulatory molecule, for example, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, MC, or a ligand that specifically binds with CD83, and the like. In an embodiment of the invention, the CAR-modified PBLs comprise an antigen binding domain, a costimulatory signaling region, and a CD3 zeta signaling domain. In an embodiment of the invention, the CAR-modified PBLs comprise a CD19-directed antigen binding domain, a 4-1BB or CD28 costimulatory signaling region, and a CD3 zeta signaling domain. In an embodiment of the invention, the CAR-modified TILs include a suicide switch (such as a Caspase-9/rimiducid) or an activation switch (such as an inducible MyD88/CD40 activation switch). In an embodiment of the invention, the CAR-modified TILs are modified using a lentiviral vector expressing a CAR.

In some embodiments of the invention, the PBLs expanded according to the methods of the present invention are used in a method to modify signaling in the cells using modified T-cell receptors (TCRs), including genetically altered TCRs. In some embodiments, the PBLs of the present invention are modified to include additional functionalities, including, but not limited to, a high-affinity T cell receptor (TCR), e.g., a TCR targeted at a tumor-associated antigen such as MAGE-1, MAGE-3, MAGE-A3, MAGE-A4, MAGE-A10, MART-1, CEA, gp100, alpha-fetoprotein (AFP), HER2, PRAME, CT83, SSX2, or NY-ESO-1. Methods for modifying TCRs and methods for creating artificial TCRs are known in the art, and are disclosed, for example, in U.S. Pat. Nos. 6,811,785; 7,569,664; 7,666,604; 8,143,376; 8,283,446; 9,181,527; 7,329,731; 7,070,995; 7,265,209; 8,361,794; and 8,697,854; and U.S. Patent Application Publication Nos. US 2017/0051036 A1; US 2010/0034834 A1; US 2011/0014169 A1; US 2016/0200824 A1; and US 2002/0058253 A1, the disclosures of each of which are incorporated by reference herein. In some embodiments of the invention, the PBLs expanded according to the methods of the present invention are used in a method to modify signaling in the cells using modified TCRs against a tumor-associated antigen. In some embodiments of the invention, the PBLs expanded according to the methods of the present invention are used in a method to modify signaling in the cells using modified TCRs, including genetically altered TCRs wherein the PBLs are modified to reduce the presence of endogenous TCRs.

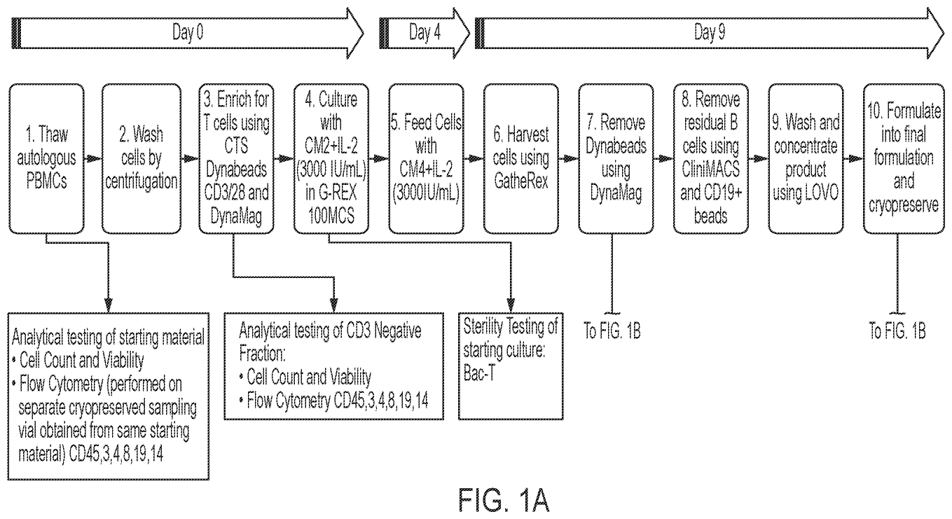

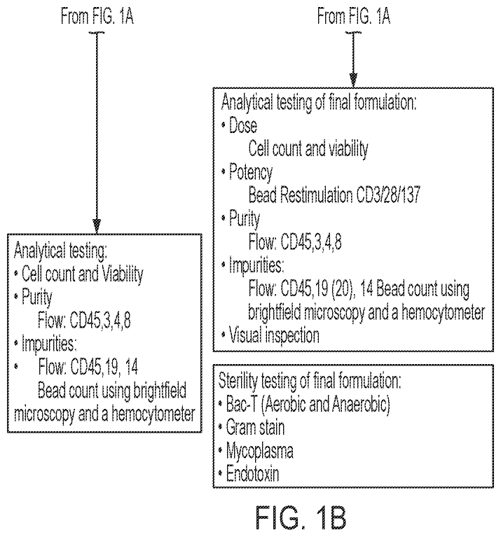

In some embodiments of the invention, the PBLs expanded according to the methods of the present invention comprise transiently or stably modified TCRs, such as TCRs modified to be specific for a cancer testis antigen, such as a MAGE-A antigen. In some embodiments, the PBLs may include at least one TCR comprising a modified complementarity determining region (CDR). In some embodiments, the PBLs may include at least one TCR comprising a modified CDR2, with retention of the wild type sequences in the beta chain to increase the TCR affinity. In some embodiments, the PBLs may include TCRs which are mutated relative to the native TCR α chain variable domain and/or β chain variable domain (see FIG. 1 b and SEQ ID NO: 2) in at least one CDR (such as CDR2), variable domain framework region, or other hypervariable regions in the variable domains of the TCRs (such as the hypervariable 4 (HV4) regions), such that the mutants produce a high affinity TCR. The PBLs may include at least one TCR anchored to the membrane by a transmembrane sequence, said TCR comprising an interchain disulfide bond between extracellular constant domain residues which is not present in native TCRs, as described in U.S. Pat. No. 8,361,794, the disclosure of which is incorporated by reference herein. In some embodiments, the PBLs may include at least one TCR having the property of binding to a specific human leukocyte antigen (HLA)-A1 complex and comprising a specified wild type TCR which has specific mutations in the TCR alpha variable domain and/or the TCR beta variable domain to increase affinity. In some embodiments of the invention, the PBLs expanded according to the methods of the present invention comprise a stably modified TCR with increased affinity to NY-ESO-1, MART-1, CEA, gp100, alpha-fetoprotein (AFP), HER2, PRAME (preferentially-expressed antigen in melanoma), CT83, SSX2, MAGE-1, MAGE-3, MAGE-A3, MAGE-A4, or MAGE-A10.

### Immune Checkpoints

In an embodiment of the present invention, one or more immune checkpoint genes may be modified. Immune checkpoints are molecules expressed by lymphocytes that regulate an immune response via inhibitory or stimulatory pathways. In the case of cancer, immune checkpoint pathways are often activated to inhibit the anti-tumor response, i.e., the expression of certain immune checkpoints by malignant cells inhibits the anti-tumor immunity and favors the growth of cancer cells. See, e.g., Marin-Acevedo et al., Journal of Hematology & Oncology (2018) 11:39. Thus, certain inhibitory checkpoint molecules serve as targets for immunotherapies of the present invention. According to particular embodiments, cells are modified through CAR or TCR to block or stimulate certain immune checkpoint pathways and thereby enhance the body's immunological activity against tumors.

The most broadly studied checkpoints include programmed cell death receptor-1 (PD-1) and cytotoxic T lymphocyte-associated molecule-4 (CTLA-4), which are inhibitory receptors on immune cells that inhibit key effector functions (e.g., activation, proliferation, cytokine release, cytotoxicity, etc.) when they interact with an inhibitory ligand. Numerous checkpoint molecules, in addition to PD-1 and CTLA-4, have emerged as potential targets for immunotherapy, as discussed in more detail below.

Non-limiting examples of immune checkpoint genes that may be silenced or inhibited include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, BAFF (BR3), CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, and GUCY1B3. For example, immune checkpoint genes that may be silenced or inhibited may be selected from the group comprising PD-1, CTLA-4, LAG-3, TIM-3, Cish, TGFβ, and PKA. BAFF (BR3) is described in Bloom, et al., J. Immunother., 2018, in press. According to another example, immune checkpoint genes that may be silenced or inhibited in TILs of the present invention may be selected from the group comprising PD-1, LAG-3, TIM-3, CTLA-4, TIGIT, CISH, TGFβR2, PRA, CBLB, BAFF (BR3), and combinations thereof.

### PD-1

One of the most studied targets for the induction of checkpoint blockade is the programmed death receptor (PD1 or PD-1, also known as PDCD1), a member of the CD28 super family of T-cell regulators. Its ligands, PD-L1 and PD-L2, are expressed on a variety of tumor cells, including melanoma. The interaction of PD-1 with PD-L1 inhibits T-cell effector function, results in T-cell exhaustion in the setting of chronic stimulation, and induces T-cell apoptosis in the tumor microenvironment. PD1 may also play a role in tumor-specific escape from immune surveillance.

According to particular embodiments, expression of PD1 in TILs is silenced or reduced in accordance with compositions and methods of the present invention.

### CTLA-4

CTLA-4 expression is induced upon T-cell activation on activated T-cells, and competes for binding with the antigen presenting cell activating antigens CD80 and CD86. Interaction of CTLA-4 with CD80 or CD86 causes T-cell inhibition and serves to maintain balance of the immune response. However, inhibition of the CTLA-4 interaction with CD80 or CD86 may prolong T-cell activation and thus increase the level of immune response to a cancer antigen.

According to particular embodiments, expression of CTLA-4 in TILs is silenced or reduced in accordance with compositions and methods of the present invention.

### LAG-3

Lymphocyte activation gene-3 (LAG-3, CD223) is expressed by T cells and natural killer (NK) cells after major histocompatibility complex (MHC) class II ligation. Although its mechanism remains unclear, its modulation causes a negative regulatory effect over T cell function, preventing tissue damage and autoimmunity. LAG-3 and PD-1 are frequently co-expressed and upregulated on TILs, leading to immune exhaustion and tumor growth. Thus, LAG-3 blockade improves anti-tumor responses. See, e.g., Marin-Acevedo et al., Journal of Hematology & Oncology (2018) 11:39.

According to particular embodiments, expression of LAG-3 in TILs is silenced or reduced in accordance with compositions and methods of the present invention.

### TIM-3

T cell immunoglobulin-3 (TIM-3) is a direct negative regulator of T cells and is expressed on NK cells and macrophages. TIM-3 indirectly promotes immunosuppression by inducing expansion of myeloid-derived suppressor cells (MDSCs). Its levels have been found to be particularly elevated on dysfunctional and exhausted T-cells, suggesting an important role in malignancy.

According to particular embodiments, expression of TIM-3 in TILs is silenced or reduced in accordance with compositions and methods of the present invention.

### Cish

Cish, a member of the suppressor of cytokine signaling (SOCS) family, is induced by TCR stimulation in CD8+ T cells and inhibits their functional avidity against tumors. Genetic deletion of Cish in CD8+ T cells may enhance their expansion, functional avidity, and cytokine polyfunctionality, resulting in pronounced and durable regression of established tumors. See, e.g., Palmer et al., Journal of Experimental Medicine, 212 (12): 2095 (2015).

According to particular embodiments, expression of Cish in TILs is silenced or reduced in accordance with compositions and methods of the present invention.

### TGFβ

The TGFβ signaling pathway has multiple functions in regulating cell growth, differentiation, apoptosis, motility and invasion, extracellular matrix production, angiogenesis, and immune response. TGFβ signaling deregulation is frequent in tumors and has crucial roles in tumor initiation, development and metastasis. At the microenvironment level, the TGFβ pathway contributes to generate a favorable microenvironment for tumor growth and metastasis throughout carcinogenesis. See, e.g., Neuzillet et al., Pharmacology & Therapeutics, Vol. 147, pp. 22-31 (2015).

According to particular embodiments, expression of TGFβ in PBLs is silenced or reduced in accordance with compositions and methods of the present invention.

### PKA

Protein Kinase A (PKA) is a well-known member of the serine-threonin protein kinase superfamily. PKA, also known as cAMP-dependent protein kinase, is a multi-unit protein kinase that mediates signal transduction of G-protein coupled receptors through its activation upon cAMP binding. It is involved in the control of a wide variety of cellular processes from metabolism to ion channel activation, cell growth and differentiation, gene expression and apoptosis. Importantly, PKA has been implicated in the initiation and progression of many tumors. See, e.g., Sapio et al., EXCLI Journal; 2014; 13: 843-855.

According to particular embodiments, expression of PKA in TILs is silenced or reduced in accordance with compositions and methods of the present invention.

### CBLB

CBLB (or CBL-B) is a E3 ubiquitin-protein ligase and is a negative regulator of T cell activation. Bachmaier, et al., Nature, 2000, 403, 211-216; Wallner, et al., Clin. Dev. Immunol. 2012, 692639.

According to particular embodiments, expression of CBLB in TILs is silenced or reduced in accordance with compositions and methods of the present invention.

Overexpression of Co-Stimulatory Receptors or Adhesion Molecules

According to additional embodiments, one or more immune checkpoint genes are enhanced. Non-limiting examples of immune checkpoint genes that may exhibit enhanced expression include certain chemokine receptors and interleukins, such as CCR2, CCR4, CCR5, CXCR2, CXCR3, CX3CR1, IL-2, IL-4, IL-7, IL-15, and IL-21.

### CCRs

For adoptive T cell immunotherapy to be effective, T cells need to be trafficked properly into tumors by chemokines. A match between chemokines secreted by tumor cells, chemokines present in the periphery, and chemokine receptors expressed by T cells is important for successful trafficking of T cells into a tumor bed.

According to particular embodiments, an increase in the expression of certain chemokine receptors in the TILs, such as one or more of CCR2, CCR4, CCR5, CXCR2, CXCR3 and CX3CR1 is contemplated. Over-expression of CCRs may help promote effector function and proliferation of TILs following adoptive transfer.

According to particular embodiments, expression of one or more of CCR2, CCR4, CCR5, CXCR2, CXCR3 and CX3CR1 is enhanced.

In an embodiment, CCR4 and/or CCR5 adhesion molecules are inserted into a TIL population using a gamma-retroviral or lentiviral method as described herein. In an embodiment, CXCR2 adhesion molecule are inserted into a TIL population using a gamma-retroviral or lentiviral method as described in Forget, et al., Frontiers Immunology 2017, 8, 908 or Peng, et al., Clin. Cancer Res. 2010, 16, 5458, the disclosures of which are incorporated by reference herein.

### Interleukins

According to additional embodiments, gene-editing methods of the present invention may be used to increase the expression of certain interleukins, such as one or more of IL-2, IL-4, IL-7, IL-15, and IL-21. Certain interleukins have been demonstrated to augment effector functions of T cells and mediate tumor control.

According to particular embodiments, expression of one or more of IL-2, IL-4, IL-7, IL-15, and IL-21 is enhanced in accordance with compositions and methods of the present invention. Aptly, the population of PBLs may be a first population, a second population and/or a third population as described herein.

Methods of Treating Cancers Including Pre-Treatment with ITK Inhibitors

The compositions and combinations of PBLs (and populations thereof) described above can be used in a method for treating hyperproliferative disorders. In a preferred embodiment, they are for use in treating cancers. In a preferred embodiment, the invention provides a method of treating a cancer, wherein the cancer is a hematological malignancy, such as a liquid tumor. In a preferred embodiment, the invention provides a method of treating a cancer, wherein the cancer is a hematological malignancy selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (or Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma, including subpopulations of patients with the foregoing diseases that are refractory to, intolerant to, or relapsed from treatment with a BTK inhibitor, including ibrutinib.

In an embodiment of the present invention, CLL patients who have been pretreated with ibrutinib represent a subpopulation of patients that can be successfully treated with the PBLs of the present invention. In particular, CLL patients who have been pretreated with ibrutinib, and who are no longer responsive to ibrutinib treatment, represent a subpopulation of patients that can be successfully treated with the PBLs of the present invention. In another embodiment, CLL patients who have been pretreated with ibrutinib and who have developed Richter's transformation (or Richter's syndrome), represent a subpopulation of patients that can be successfully treated with the PBLs of the present invention. In another embodiment, CLL patients who have been pretreated with ibrutinib, who have developed Richter's transformation (or Richter's syndrome) and who are no longer responsive to ibrutinib treatment, represent a subpopulation of patients that can be successfully treated with the PBLs of the present invention.

In an embodiment, the invention provides a method of treating a cancer, wherein the cancer is a hematological malignancy that responds to therapy with PD-1 and/or PD-L1 inhibitors including pembrolizumab, nivolumab, durvalumab, avelumab, or atezolizumab.

In an embodiment, the invention provides a method of treating a cancer in a patient with a population of PBLs comprising the steps of:
(a) obtaining peripheral blood mononuclear cells (PBMCs) from less than or equal to about 50 mL of whole blood from the patient, wherein the patient is optionally pretreated with an ITK inhibitor;
(b) admixing beads selective for CD3 and CD28 with the PBMCs, wherein the beads are added at a ratio of 3 beads:1 cell, to form an admixture of PBMCs and beads;
(c) culturing the admixture of PBMCs and beads at a density of about 25,000 cells per cm² to about 50,000 cells per cm² on a gas-permeable surface of one or more containers containing a first cell culture medium and IL-2 for a period of about 4 days;
(d) adding to each container of step (c) IL-2 and a second cell culture medium that is the same as or different from the first cell culture medium and culturing for a period of about 5 days to about 7 days to form an expanded population of PBLs;
(e) harvesting from each container the expanded population of PBLs;
(f) removing residual beads from the harvested population of PBLs to provide a PBL product;
(f) formulating and optionally cryopreserving the PBL product; and
(g) administering to the patient a therapeutically effective amount of the PBL product,
wherein the ITK inhibitor is optionally an ITK inhibitor that covalently binds to ITK.

In an embodiment, the invention provides a method of treating a cancer in a patient with a population of PBLs comprising the steps of:
(a) obtaining peripheral blood mononuclear cells (PBMCs) from less than or equal to about 50 mL of whole blood from the patient, wherein the patient is optionally pretreated with an ITK inhibitor;
(b) admixing beads selective for CD3 and CD28 with the PBMCs, wherein the beads are added at a ratio of 3 beads:1 cell, to form an admixture of PBMCs and beads;
(c) culturing the admixture of PBMCs and beads at a density of about 25,000 cells per cm² to about 50,000 cells per cm² on a gas-permeable surface of one or more containers containing a first cell culture medium and IL-2 for a period of about 4 days;
(d) adding to each container of step (c) IL-2 and a second cell culture medium that is the same as or different from the first cell culture medium and culturing for a period of about 5 days to about 7 days to form an expanded population of PBLs;
(e) harvesting from each container the expanded population of PBLs;
(f) removing residual beads from the harvested population of PBLs to provide a PBL product;
(i) formulating and optionally cryopreserving the PBL product; and
(j) administering to the patient a therapeutically effective amount of the PBL product,
   wherein the ITK inhibitor is optionally an ITK inhibitor that covalently binds to ITK, and wherein the cancer is a hematological malignancy selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (or Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma.

In an embodiment, the invention provides a method of treating a cancer in a patient with a population of PBLs comprising:
a. Obtaining a sample of peripheral blood mononuclear cells (PBMCs) from the peripheral blood of a patient, wherein said sample is optionally cryopreserved and the patient is optionally pretreated with an ITK inhibitor;
b. Optionally washing the PBMCs by centrifugation;
c. Admixing magnetic beads selective for CD3 and CD28 to the PBMCs to form an admixture of the beads and the PBMCs;
d. Seeding the admixture of the beads and the PBMCs into a gas-permeable container and co-culturing said PBMCs in media comprising about 3000 IU/mL of IL-2 in for about 4 to about 6 days;
e. Feeding said PBMCs using media comprising about 3000 IU/mL of IL-2, and co-culturing said PBMCs for about 5 days, such that the total co-culture period of steps d and e is about 9 to about 11 days;
f. Harvesting PBMCs from media;
g. Removing residual magnetic beads selective for CD3 and CD28 from the harvested PBMCs using a magnet;
h. Removing residual B-cells from the harvested PBMCs using magnetic-activated cell sorting and beads selective for CD19 to provide a PBL product;
i. Washing and concentrating the PBL product using a cell harvester;
j. Formulating and optionally cryopreserving the PBL product; and
k. Administering to the patient a therapeutically effective amount of the PBL product, wherein the ITK inhibitor is optionally an ITK inhibitor that covalently binds to ITK.

In an embodiment, the invention provides a method of treating a cancer in a patient with a population of PBLs comprising:
a. Obtaining a sample of peripheral blood mononuclear cells (PBMCs) from the peripheral blood of a patient, wherein said sample is optionally cryopreserved and the patient is optionally pretreated with an ITK inhibitor;
b. Optionally washing the PBMCs by centrifugation;
c. Admixing magnetic beads selective for CD3 and CD28 to the PBMCs to form an admixture of the beads and the PBMCs;
d. Seeding the admixture of the beads and the PBMCs into a gas-permeable container and co-culturing said PBMCs in media comprising about 3000 IU/mL of IL-2 in for about 4 to about 6 days;
e. Feeding said PBMCs using media comprising about 3000 IU/mL of IL-2, and co-culturing said PBMCs for about 5 days, such that the total co-culture period of steps d and e is about 9 to about 11 days;
f. Harvesting PBMCs from media;
g. Removing residual magnetic beads selective for CD3 and CD28 from the harvested PBMCs using a magnet;
h. Removing residual B-cells from the harvested PBMCs using magnetic-activated cell sorting and beads selective for CD19 to provide a PBL product;
i. Washing and concentrating the PBL product using a cell harvester;
j. Formulating and optionally cryopreserving the PBL product; and administering to the patient an effective amount of the PBL product, wherein the ITK inhibitor is optionally an ITK inhibitor that covalently binds to ITK, and wherein the cancer is a hematological malignancy selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (or Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma.

In an embodiment, the invention provides the method of treating cancer in a patient described in any of the preceding paragraphs as applicable above modified such that before the step of admixing beads selective for CD3 and CD28 with the PBMCs the method further comprises performing the step of removing B-cells from the PBMCs to provide PBMCs depleted of B-cells.

In an embodiment, the invention provides the method of treating cancer in a patient described in any of the preceding paragraphs as applicable above modified such that before the step of admixing beads selective for CD3 and CD28 with the PBMCs the method further comprises performing the steps of: (i) determining the proportion of the PMBCs constituted by B-cells as a B-cell percentage; and (ii) if the B-cell percentage determined in step (i) is at least about seventy percent (70%), removing B-cells from the PBMCs by selecting against CD19 to provide PBMCs depleted of B-cells.

In an embodiment, the invention provides any of the method of treating cancer in a patient described in any of the preceding paragraphs as applicable above modified such that if the B-cell percentage is at least about 75% the B-cell removal step is performed.

In an embodiment, the invention provides the method of treating cancer in a patient described in any of the preceding paragraphs as applicable above modified such that if the B-cell percentage is at least about 80% the B-cell removal step is performed.

In an embodiment, the invention provides the method of treating cancer in a patient described in any of the preceding paragraphs as applicable above modified such that if the B-cell percentage is at least about 85% the B-cell removal step is performed.

In an embodiment, the invention provides the method of treating cancer in a patient described in any of the preceding paragraphs as applicable above modified such that if the B-cell percentage is at least about 50%, 55%, 60%, 65%, 70%, 75%, 80%, 85%, 90%, or 95% the B-cell removal step is performed.

In an embodiment of the invention, the invention provides the method of treating cancer in a patient described in any of the preceding paragraphs as applicable above modified such that removal of B-cells, or B-cell depletion (BCD), occurs on Day 0 or on Day 9 of a 9-day expansion process. In another embodiment, the BCD occurs on both Day 0 and Day 9 of a 9-day expansion process. In an embodiment of the invention, BCD occurs on Day 0 or Day 11 of an 11-day expansion process. In another embodiment, the BCD occurs on both Day 0 and Day 11 of an 11-day expansion process.

In an embodiment of the invention, the invention provides the method of treating cancer in a patient described in any of the preceding paragraphs as applicable above modified such that the BCD step is performed on a PBMC sample from a patient having a high initial B-cell count. In one embodiment, a high initial B-cell count is about 50%, 55%, 60%, 65%, 70%, 75%, 80%, 85%, 90%, 95%, or more B-cells in the initial PBMC sample.

In an embodiment, the invention provides the method of treating cancer in a patient described in any of the preceding paragraphs as applicable above modified such that the B-cell percentage is determined by comparison of the CD19+ cells to the CD45+ cells in the PBMCs.

In an embodiment, the invention provides the method of treating cancer in a patient described in any of the preceding paragraphs as applicable above modified such that the B-cell percentage is determined by comparison of the fraction of CD19+/CD45+ cells to the fraction of CD45+ cells in the PBMCs.

In an embodiment, the invention provides the method of treating cancer in a patient described in any of the preceding paragraphs as applicable above modified such that the comparison of the fraction of CD19+ cells to the fraction of CD45+ cells in the PBMCs is performed by contacting the PBMCs with a CD19 stain and a CD45 stain, and then comparing the subpopulation of PBMCs positive for the both CD19 stain and the CD45 stain with the subpopulation of PBMCs positive for only the CD19 stain.

In an embodiment, the invention provides the method of treating cancer in a patient described in any of the preceding paragraphs as applicable above modified such that the CD19 stain is an anti-CD19 antibody conjugated to a first label and the CD45 stain is an anti-CD45 antibody conjugated to a second label.

In an embodiment, the invention provides the method of treating cancer in a patient described in any of the preceding paragraphs as applicable above modified such that the first label is a first fluorochrome and the second label is a second fluorochrome that is different from the first fluorochrome.

In an embodiment, the invention provides the method of treating cancer in a patient described in any of the preceding paragraphs as applicable above modified such that the step of removing B-cells from the PBMCs is performed by selecting against CD19 to provide PBMCs depleted of B-cells.

In an embodiment, the invention provides the method of treating cancer in a patient described in any of the preceding paragraphs as applicable above modified such that the step of removing B-cells from the PBMCs is performed by admixing beads selective for CD19 to the PBMCs to form complexes of the beads and CD19+ cells and removing the complexes from the PBMCs to provide PBMCs depleted of B-cells.

In an embodiment, the invention provides the method of treating cancer in a patient described in any of the preceding paragraphs as applicable above modified such that the step of removing B-cells from the PBMCs is performed by admixing magnetic beads selective for CD19 to the PBMCs to form complexes of the magnetic beads and CD19+ cells and using a magnet to remove the complexes from the PBMCs to provide PBMCs depleted of B-cells.

In an embodiment, the invention provides a pharmaceutical composition for use in a method of treating a cancer in a patient comprising the steps of:
(a) obtaining peripheral blood mononuclear cells (PBMCs) from less than or equal to about 50 mL of whole blood from the patient, wherein the patient is optionally pretreated with an ITK inhibitor;
(b) admixing beads selective for CD3 and CD28 with the PBMCs, wherein the beads are added at a ratio of 3 beads:1 cell, to form an admixture of PBMCs and beads;
(c) culturing the admixture of PBMCs and beads at a density of about 25,000 cells per cm² to about 50,000 cells per cm² on a gas-permeable surface of one or more containers containing a first cell culture medium and IL-2 for a period of about 4 days;
(d) adding to each container of step (c) IL-2 and a second cell culture medium that is the same as or different from the first cell culture medium and culturing for a period of about 5 days to about 7 days to form an expanded population of PBLs;
(e) harvesting from each container the expanded population of PBLs;
(f) removing residual beads from the harvested population of PBLs to provide a PBL product;
(h) formulating the PBL product to form a pharmaceutical composition and optionally cryopreserving the pharmaceutical composition; and
(i) administering to the patient a therapeutically effective amount of the pharmaceutical composition, wherein the ITK inhibitor is optionally an ITK inhibitor that covalently binds to ITK.

In an embodiment, the invention provides a pharmaceutical composition for use in a method of treating a cancer in a patient comprising the steps of:
(a) obtaining peripheral blood mononuclear cells (PBMCs) from less than or equal to about 50 mL of whole blood from the patient, wherein the patient is optionally pretreated with an ITK inhibitor;
(b) admixing beads selective for CD3 and CD28 with the PBMCs, wherein the beads are added at a ratio of 3 beads:1 cell, to form an admixture of PBMCs and beads;
(c) culturing the admixture of PBMCs and beads at a density of about 25,000 cells per cm² to about 50,000 cells per cm² on a gas-permeable surface of one or more containers containing a first cell culture medium and IL-2 for a period of about 4 days;
(d) adding to each container of step (c) IL-2 and a second cell culture medium that is the same as or different from the first cell culture medium and culturing for a period of about 5 days to about 7 days to form an expanded population of PBLs;
(e) harvesting from each container the expanded population of PBLs;
(f) removing residual beads from the harvested population of PBLs to provide a PBL product;
(g) formulating the PBL product to form a pharmaceutical composition and optionally cryopreserving the pharmaceutical composition; and administering to the patient a therapeutically effective amount of the pharmaceutical composition, wherein the ITK inhibitor is optionally an ITK inhibitor that covalently binds to ITK, and wherein the cancer is a hematological malignancy selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (or Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma.

In an embodiment, the invention provides a pharmaceutical composition for use in a method of treating a cancer in a patient comprising the steps of:
(a) Obtaining a sample of peripheral blood mononuclear cells (PBMCs) from the peripheral blood of a patient, wherein said sample is optionally cryopreserved and the patient is optionally pretreated with an ITK inhibitor;
(b) Optionally washing the PBMCs by centrifugation;
(c) Admixing magnetic beads selective for CD3 and CD28 to the PBMCs to form an admixture of the beads and the PBMCs;
(d) Seeding the admixture of the beads and the PBMCs into a gas-permeable container and co-culturing said PBMCs in media comprising about 3000 IU/mL of IL-2 in for about 4 to about 6 days;
(e) Feeding said PBMCs using media comprising about 3000 IU/mL of IL-2, and co-culturing said PBMCs for about 5 days, such that the total co-culture period of steps d and e is about 9 to about 11 days;
(f) Harvesting PBMCs from media;
(g) Removing residual magnetic beads selective for CD3 and CD28 from the harvested PBMCs using a magnet;
(h) Removing residual B-cells from the harvested PBMCs using magnetic-activated cell sorting and beads selective for CD19 to provide a PBL product;
(i) Washing and concentrating the PBL product using a cell harvester;
(j) Formulating the PBL product to form a pharmaceutical composition and optionally cryopreserving the pharmaceutical composition; and
(k) Administering to the patient a therapeutically effective amount of the pharmaceutical composition, wherein the ITK inhibitor is optionally an ITK inhibitor that covalently binds to ITK.

In an embodiment, the invention provides a pharmaceutical composition for use in a method of treating a cancer in a patient comprising the steps of:
(b) Obtaining a sample of peripheral blood mononuclear cells (PBMCs) from the peripheral blood of a patient, wherein said sample is optionally cryopreserved and the patient is optionally pretreated with an ITK inhibitor;
(b) Optionally washing the PBMCs by centrifugation;
(c) Admixing magnetic beads selective for CD3 and CD28 to the PBMCs to form an admixture of the beads and the PBMCs;
(d) Seeding the admixture of the beads and the PBMCs into a gas-permeable container and co-culturing said PBMCs in media comprising about 3000 IU/mL of IL-2 in for about 4 to about 6 days;
(e) Feeding said PBMCs using media comprising about 3000 IU/mL of IL-2, and co-culturing said PBMCs for about 5 days, such that the total co-culture period of steps d and e is about 9 to about 11 days;
(f) Harvesting PBMCs from media;
(g) Removing residual magnetic beads selective for CD3 and CD28 from the harvested PBMCs using a magnet;
(h) Removing residual B-cells from the harvested PBMCs using magnetic-activated cell sorting and beads selective for CD19 to provide a PBL product;
(i) Washing and concentrating the PBL product using a cell harvester;
(j) Formulating the PBL product to form a pharmaceutical composition and optionally cryopreserving the pharmaceutical composition; and
(k) Administering to the patient a therapeutically effective amount of the pharmaceutical composition, wherein the ITK inhibitor is optionally an ITK inhibitor that covalently binds to ITK, and wherein the cancer is a hematological malignancy selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (or Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma.

In an embodiment, the invention provides the pharmaceutical composition for use in a method of treating a cancer in a patient described in any of the preceding paragraphs as applicable above modified such that before the step of admixing beads selective for CD3 and CD28 with the PBMCs the method further comprises performing the step of removing B-cells from the PBMCs to provide PBMCs depleted of B-cells.

In an embodiment, the invention provides the pharmaceutical composition for use in a method of treating a cancer in a patient described in any of the preceding paragraphs as applicable above modified such that before the step of admixing beads selective for CD3 and CD28 with the PBMCs the method further comprises performing the steps of: (i) determining the proportion of the PMBCs constituted by B-cells as a B-cell percentage; and (ii) if the B-cell percentage determined in step (i) is at least about seventy percent (70%), removing B-cells from the PBMCs by selecting against CD19 to provide PBMCs depleted of B-cells.

In an embodiment, the invention provides the pharmaceutical composition for use in a method of treating a cancer in a patient described in any of the preceding paragraphs as applicable above modified such that if the B-cell percentage is at least about 75% the B-cell removal step is performed.

In an embodiment, the invention provides the pharmaceutical composition for use in a method of treating a cancer in a patient described in any of the preceding paragraphs as applicable above modified such that if the B-cell percentage is at least about 80% the B-cell removal step is performed.

In an embodiment, the invention provides the pharmaceutical composition for use in a method of treating a cancer in a patient described in any of the preceding paragraphs as applicable above modified such that if the B-cell percentage is at least about 85% the B-cell removal step is performed.

In an embodiment, the invention provides the pharmaceutical composition for use in a method of treating a cancer in a patient described in any of the preceding paragraphs as applicable above modified such that if the B-cell percentage is at least about 50%, 55%, 60%, 65%, 70%, 75%, 80%, 85%, 90%, or 95% the B-cell removal step is performed.

In an embodiment of the invention, the invention provides the pharmaceutical composition for use in a method of treating a cancer in a patient described in any of the preceding paragraphs as applicable above modified such that removal of B-cells, or B-cell depletion (BCD), occurs on Day 0 or on Day 9 of a 9-day expansion process. In another embodiment, the BCD occurs on both Day 0 and Day 9 of a 9-day expansion process. In an embodiment of the invention, BCD occurs on Day 0 or Day 11 of an 11-day expansion process. In another embodiment, the BCD occurs on both Day 0 and Day 11 of an 11-day expansion process.

In an embodiment of the invention, the invention provides the pharmaceutical composition for use in a method of treating a cancer in a patient described in any of the preceding paragraphs as applicable above modified such that the BCD step is performed on a PBMC sample from a patient having a high initial B-cell count. In one embodiment, a high initial B-cell count is about 50%, 55%, 60%, 65%, 70%, 75%, 80%, 85%, 90%, 95%, or more B-cells in the initial PBMC sample.

In an embodiment, the invention provides the pharmaceutical composition for use in a method of treating a cancer in a patient described in any of the preceding paragraphs as applicable above modified such that the B-cell percentage is determined by comparison of the CD19+ cells to the CD45+ cells in the PBMCs.

In an embodiment, the invention provides the pharmaceutical composition for use in a method of treating a cancer in a patient described in any of the preceding paragraphs as applicable above modified such that the B-cell percentage is determined by comparison of the fraction of CD19+/CD45+ cells to the fraction of CD45+ cells in the PBMCs.

In an embodiment, the invention provides the pharmaceutical composition for use in a method of treating a cancer in a patient described in any of the preceding paragraphs as applicable above modified such that the comparison of the fraction of CD19+ cells to the fraction of CD45+ cells in the PBMCs is performed by contacting the PBMCs with a CD19 stain and a CD45 stain, and then comparing the subpopulation of PBMCs positive for the both CD19 stain and the CD45 stain with the subpopulation of PBMCs positive for only the CD19 stain.

In an embodiment, the invention provides the pharmaceutical composition for use in a method of treating a cancer in a patient described in any of the preceding paragraphs as applicable above modified such that the CD19 stain is an anti-CD19 antibody conjugated to a first label and the CD45 stain is an anti-CD45 antibody conjugated to a second label.

In an embodiment, the invention provides the pharmaceutical composition for use in a method of treating a cancer in a patient described in any of the preceding paragraphs as applicable above modified such that the first label is a first fluorochrome and the second label is a second fluorochrome that is different from the first fluorochrome.

In an embodiment, the invention provides the pharmaceutical composition for use in a method of treating a cancer in a patient described in any of the preceding paragraphs as applicable above modified such that the step of removing B-cells from the PBMCs is performed by selecting against CD19 to provide PBMCs depleted of B-cells.

In an embodiment, the invention provides the pharmaceutical composition for use in a method of treating a cancer in a patient described in any of the preceding paragraphs as applicable above modified such that the step of removing B-cells from the PBMCs is performed by admixing beads selective for CD19 to the PBMCs to form complexes of the beads and CD19+ cells and removing the complexes from the PBMCs to provide PBMCs depleted of B-cells.

In an embodiment, the invention provides the pharmaceutical composition for use in a method of treating a cancer in a patient described in any of the preceding paragraphs as applicable above modified such that the step of removing B-cells from the PBMCs is performed by admixing magnetic beads selective for CD19 to the PBMCs to form complexes of the magnetic beads and CD19+ cells and using a magnet to remove the complexes from the PBMCs to provide PBMCs depleted of B-cells.

In an embodiment, the invention provides the use of a pharmaceutical composition in a method for the treatment of a cancer in a patient, the method comprising the steps of:
(a) obtaining peripheral blood mononuclear cells (PBMCs) from less than or equal to about 50 mL of whole blood from the patient, wherein the patient is optionally pretreated with an ITK inhibitor;
(b) admixing beads selective for CD3 and CD28 with the PBMCs, wherein the beads are added at a ratio of 3 beads:1 cell, to form an admixture of PBMCs and beads;
(c) culturing the admixture of PBMCs and beads at a density of about 25,000 cells per cm² to about 50,000 cells per cm² on a gas-permeable surface of one or more containers containing a first cell culture medium and IL-2 for a period of about 4 days;
(d) adding to each container of step (c) IL-2 and a second cell culture medium that is the same as or different from the first cell culture medium and culturing for a period of about 5 days to about 7 days to form an expanded population of PBLs;
(e) harvesting from each container the expanded population of PBLs;
(f) removing the beads from the harvested population of PBLs to provide a PBL product;
(g) formulating the PBL product to form a pharmaceutical composition and optionally cryopreserving the pharmaceutical composition; and
(h) administering to the patient a therapeutically effective amount of the pharmaceutical composition,
   wherein the ITK inhibitor is optionally an ITK inhibitor that covalently binds to ITK.

In an embodiment, the invention provides the use of a pharmaceutical composition in a method for the treatment of a cancer in a patient, the method comprising the steps of:
(a) obtaining peripheral blood mononuclear cells (PBMCs) from less than or equal to about 50 mL of whole blood from the patient, wherein the patient is optionally pretreated with an ITK inhibitor;
(b) admixing beads selective for CD3 and CD28 with the PBMCs, wherein the beads are added at a ratio of 3 beads:1 cell, to form an admixture of PBMCs and beads;
(c) culturing the admixture of PBMCs and beads at a density of about 25,000 cells per cm² to about 50,000 cells per cm² on a gas-permeable surface of one or more containers containing a first cell culture medium and IL-2 for a period of about 4 days;
(d) adding to each container of step (c) IL-2 and a second cell culture medium that is the same as or different from the first cell culture medium and culturing for a period of about 5 days to about 7 days to form an expanded population of PBLs;
(e) harvesting from each container the expanded population of PBLs;
(f) removing the beads from the harvested population of PBLs to provide a PBL product;
(g) formulating the PBL product to form a pharmaceutical composition and optionally cryopreserving the pharmaceutical composition; and
(h) administering to the patient a therapeutically effective amount of the pharmaceutical composition,
   wherein the ITK inhibitor is optionally an ITK inhibitor that covalently binds to ITK, and wherein the cancer is a hematological malignancy selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (or Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma.

In an embodiment, the invention provides the use of a pharmaceutical composition in a method of treating cancer in a patient, the method comprising the steps of:

(a) Obtaining a sample of peripheral blood mononuclear cells (PBMCs) from the peripheral blood of a patient, wherein said sample is optionally cryopreserved and the patient is optionally pretreated with an ITK inhibitor;
(b) Optionally washing the PBMCs by centrifugation;
(c) Adding magnetic beads selective for CD3 and CD28 to the PBMCs;
(d) Seeding PBMCs into a gas-permeable container and co-culturing said PBMCs in media comprising about 3000 IU/mL of IL-2 in for about 4 to about 6 days;
(e) Feeding said PBMCs using media comprising about 3000 IU/mL of IL-2, and co-culturing said PBMCs for about 5 days, such that the total co-culture period of steps d and e is about 9 to about 11 days;
(f) Harvesting PBMCs from media;
(g) Removing residual magnetic beads selective for CD3 and CD28 from the harvested PBMCs using a magnet;
(h) Removing residual B-cells from the harvested PBMCs using magnetic-activated cell sorting and CD19⁺ beads to provide a PBL product;
(i) Washing and concentrating the PBL product using a cell harvester;
(j) Formulating the PBL product to form a pharmaceutical composition and optionally cryopreserving the pharmaceutical composition; and
(k) Administering to the patient a therapeutically effective amount of the pharmaceutical composition, wherein the ITK inhibitor is optionally an ITK inhibitor that covalently binds to ITK.

In an embodiment, the invention provides the use of a pharmaceutical composition in a method of treating cancer in a patient, the method comprising the steps of:

(a) Obtaining a sample of peripheral blood mononuclear cells (PBMCs) from the peripheral blood of a patient, wherein said sample is optionally cryopreserved and the patient is optionally pretreated with an ITK inhibitor;
(b) Optionally washing the PBMCs by centrifugation;
(c) Adding magnetic beads selective for CD3 and CD28 to the PBMCs;
(d) Seeding PBMCs into a gas-permeable container and co-culturing said PBMCs in media comprising about 3000 IU/mL of IL-2 in for about 4 to about 6 days;
(e) Feeding said PBMCs using media comprising about 3000 IU/mL of IL-2, and co-culturing said PBMCs for about 5 days, such that the total co-culture period of steps d and e is about 9 to about 11 days;
(f) Harvesting PBMCs from media;
(g) Removing the magnetic beads selective for CD3 and CD28 using a magnet;
(h) Removing residual B-cells using magnetic-activated cell sorting and CD19⁺ beads to provide a PBL product;
(i) Washing and concentrating the PBL product using a cell harvester;
(j) Formulating the PBL product to form a pharmaceutical composition and optionally cryopreserving the pharmaceutical composition; and
(k) Administering to the patient a therapeutically effective amount of the pharmaceutical composition, wherein the ITK inhibitor is optionally an ITK inhibitor that covalently binds to ITK, and wherein the cancer is a hematological malignancy selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (or Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma.

In an embodiment, the invention provides the use of a pharmaceutical composition in a method of treating cancer in a patient described in any of the preceding paragraphs as applicable above modified such that before the step of admixing beads selective for CD3 and CD28 with the PBMCs the method further comprises performing the step of removing B-cells from the PBMCs to provide PBMCs depleted of B-cells.

In an embodiment, the invention provides the use of a pharmaceutical composition in a method of treating cancer in a patient described in any of the preceding paragraphs as applicable above modified such that before the step of admixing beads selective for CD3 and CD28 with the PBMCs the method further comprises performing the steps of: (i) determining the proportion of the PMBCs constituted by B-cells as a B-cell percentage; and (ii) if the B-cell percentage determined in step (i) is at least about seventy percent (70%), removing B-cells from the PBMCs by selecting against CD19 to provide PBMCs depleted of B-cells.

In an embodiment, the invention provides the use of a pharmaceutical composition in a method of treating cancer in a patient described in any of the preceding paragraphs as applicable above modified such that if the B-cell percentage is at least about 75% the B-cell removal step is performed.

In an embodiment, the invention provides the use of a pharmaceutical composition in a method of treating cancer in a patient described in any of the preceding paragraphs as applicable above modified such that if the B-cell percentage is at least about 80% the B-cell removal step is performed.

In an embodiment, the invention provides the use of a pharmaceutical composition in a method of treating cancer in a patient described in any of the preceding paragraphs as applicable above modified such that if the B-cell percentage is at least about 85% the B-cell removal step is performed.

In an embodiment, the invention provides the use of a pharmaceutical composition in a method of treating cancer in a patient described in any of the preceding paragraphs as applicable above modified such that if the B-cell percentage is at least about 50%, 55%, 60%, 65%, 70%, 75%, 80%, 85%, 90%, or 95% the B-cell removal step is performed.

In an embodiment, the invention provides the use of a pharmaceutical composition in a method of treating cancer in a patient described in any of the preceding paragraphs as applicable above modified such that the B-cell percentage is determined by comparison of the CD19+ cells to the CD45+ cells in the PBMCs.

In an embodiment of the invention, the invention provides the use of a pharmaceutical composition in a method of treating cancer in a patient described in any of the preceding paragraphs as applicable above modified such that removal of B-cells, or B-cell depletion (BCD), occurs on Day 0 or on Day 9 of a 9-day expansion process. In another embodiment, the BCD occurs on both Day 0 and Day 9 of a 9-day expansion process. In an embodiment of the invention, BCD occurs on Day 0 or Day 11 of an 11-day expansion process. In another embodiment, the BCD occurs on both Day 0 and Day 11 of an 11-day expansion process.

In an embodiment of the invention, the invention provides the use of a pharmaceutical composition in a method of treating cancer in a patient described in any of the preceding paragraphs as applicable above modified such that the BCD step is performed on a PBMC sample from a patient having a high initial B-cell count. In one embodiment, a high initial B-cell count is about 50%, 55%, 60%, 65%, 70%, 75%, 80%, 85%, 90%, 95%, or more B-cells in the initial PBMC sample.

In an embodiment, the invention provides the use of a pharmaceutical composition in a method of treating cancer in a patient described in any of the preceding paragraphs as applicable above modified such that the B-cell percentage is determined by comparison of the fraction of CD19+/CD45+ cells to the fraction of CD45+ cells in the PBMCs.

In an embodiment, the invention provides the use of a pharmaceutical composition in a method of treating cancer in a patient described in any of the preceding paragraphs as applicable above modified such that the comparison of the fraction of CD19+ cells to the fraction of CD45+ cells in the PBMCs is performed by contacting the PBMCs with a CD19 stain and a CD45 stain, and then comparing the subpopulation of PBMCs positive for the both CD19 stain and the CD45 stain with the subpopulation of PBMCs positive for only the CD19 stain.

In an embodiment, the invention provides the use of a pharmaceutical composition in a method of treating cancer in a patient described in any of the preceding paragraphs as applicable above modified such that the CD19 stain is an anti-CD19 antibody conjugated to a first label and the CD45 stain is an anti-CD45 antibody conjugated to a second label.

In an embodiment, the invention provides the use of a pharmaceutical composition in a method of treating cancer in a patient described in any of the preceding paragraphs as applicable above modified such that the first label is a first fluorochrome and the second label is a second fluorochrome that is different from the first fluorochrome.

In an embodiment, the invention provides the use of a pharmaceutical composition in a method of treating cancer in a patient described in any of the preceding paragraphs as applicable above modified such that before the step of admixing beads selective for CD3 and CD28 with the PBMCs the method further comprises performing the step of removing B-cells from the PBMCs by selecting against CD19 to provide PBMCs depleted of B-cells.

In an embodiment, the invention provides the use of a pharmaceutical composition in a method of treating cancer in a patient described in any of the preceding paragraphs as applicable above modified such that before the step of admixing beads selective for CD3 and CD28 with the PBMCs the method further comprises performing the step of removing B-cells from the PBMCs by admixing beads selective for CD19 with the PBMCs to form complexes of the beads and CD19+ cells in an admixture and removing the complexes from the admixture to provide PBMCs depleted of B-cells.

In an embodiment, the invention provides the use of a pharmaceutical composition in a method of treating cancer in a patient described in any of the preceding paragraphs as applicable above modified such that before the step of admixing beads selective for CD3 and CD28 with the PBMCs the method further comprises performing the step of removing B-cells from the PBMCs by admixing magnetic beads selective for CD19 with the PBMCs to form complexes of the magnetic beads and CD19+ cells in an admixture and using a magnet to remove the complexes from the admixture to provide PBMCs depleted of B-cells.

In any of the foregoing embodiments of the invention, pre-treatment with a kinase inhibitor is described. In an embodiment, the kinase inhibitor is selected from the group consisting of imatinib, dasatinib, ibrutinib, bosutinib, nilotinib, erlotinib, or other kinase inhibitors, tyrosine kinase inhibitors, or serine/threonine kinase inhibitors known in the art. In an embodiment, pre-treatment regimens with a kinase inhibitor are as known in the art and/or as prescribed by a physician.

In any of the foregoing embodiments of the invention, pre-treatment with an IL inducible T-cell kinase (ITK) inhibitor is described. Interleukin-2-inducible T cell kinase (ITK) is a non-receptor tyrosine kinase expressed in T-cells and regulates various pathways. Any ITK inhibitor known in the art may be used in embodiments of the present invention (see, for example, Lo, et al, Expert Opinion on Therapeutic Patents, 20:459-469 (2010); Vargas, et al, Scandinavian Journal of Immunology, 78(2):130-139 (2013); WO2015112847; WO2016118951; WO2007136790, US20120058984A1, and U.S. Pat. Nos. 9,531,689 and 9,695,200; all of which are incorporated by reference herein in their entireties). In an embodiment of the invention, the ITK inhibitor is a covalent ITK inhibitor that covalently and irreversibly binds to ITK. In an embodiment of the invention, the ITK inhibitor is an allosteric ITK inhibitor that binds to ITK. In an embodiment of the invention, the ITK inhibitor is selected from the group consisting of aminothiazole-based ITK inhibitors, 5-aminomethylbenzimdazoles-based ITK inhibitors, 3-Aminopyrid-2-ones-based ITK inhibitors, (4 or 5-aryl)pyrazolyl-indole-based ITK inhibitors, benzimidazole-based ITK inhibitors, aminobenzimidazole-based ITK inhibitors, aminopyrimidine-based ITK inhibitors, aminopyridine-based ITK inhibitors, diazolodiazine-based ITK inhibitors, triazole-based ITK inhibitors, 3-aminopyride-2-ones-based ITK inhibitors, indolylindazole-based ITK inhibitors, indole-based ITK inhibitors, aza-indole-based ITK inhibitors, pyrazolyl-indole-based inhibitors, thienopyrazole-based ITK inhibitors, heterocyclic ITK inhibitors, and ITK inhibitors targeting cysteine-442 in the ATP pocket (such as ibrutinib), aza-benzimidazole-based ITK inhibitors, benzothiazole-based ITK inhibitors, indole-based ITK inhibitors, pyridone-based ITK inhibitors, sulfoximine-substituted pyrimidine ITK inhibitors, arylpyridinone-based ITK inhibitors, and any other ITK inhibitors known in the art. In an embodiment of the invention, pre-treatment regimens with an ITK inhibitor are as known in the art and/or as prescribed by a physician. In an embodiment of the invention, the ITK inhibitor is selected from the group consisting of:

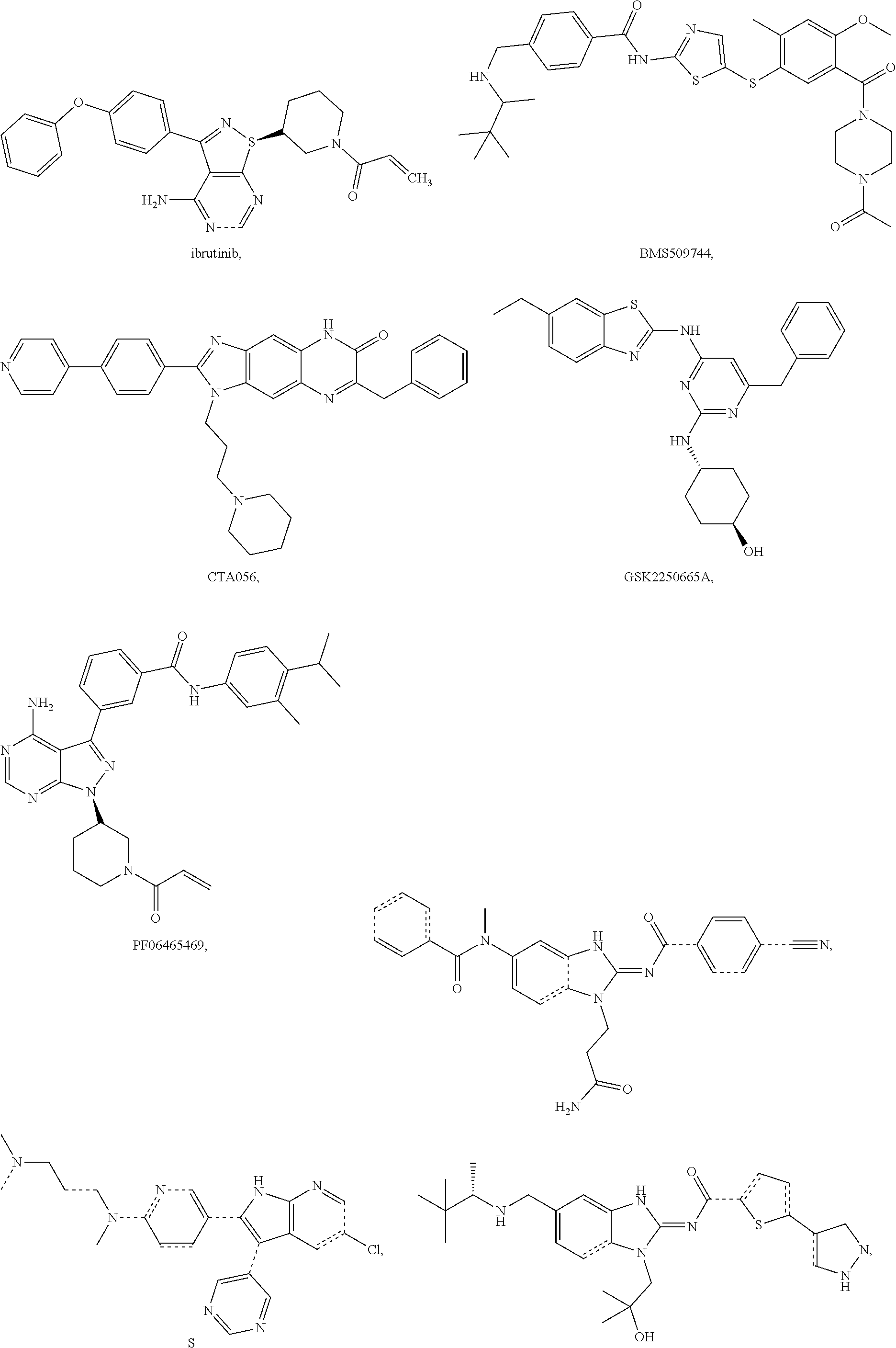

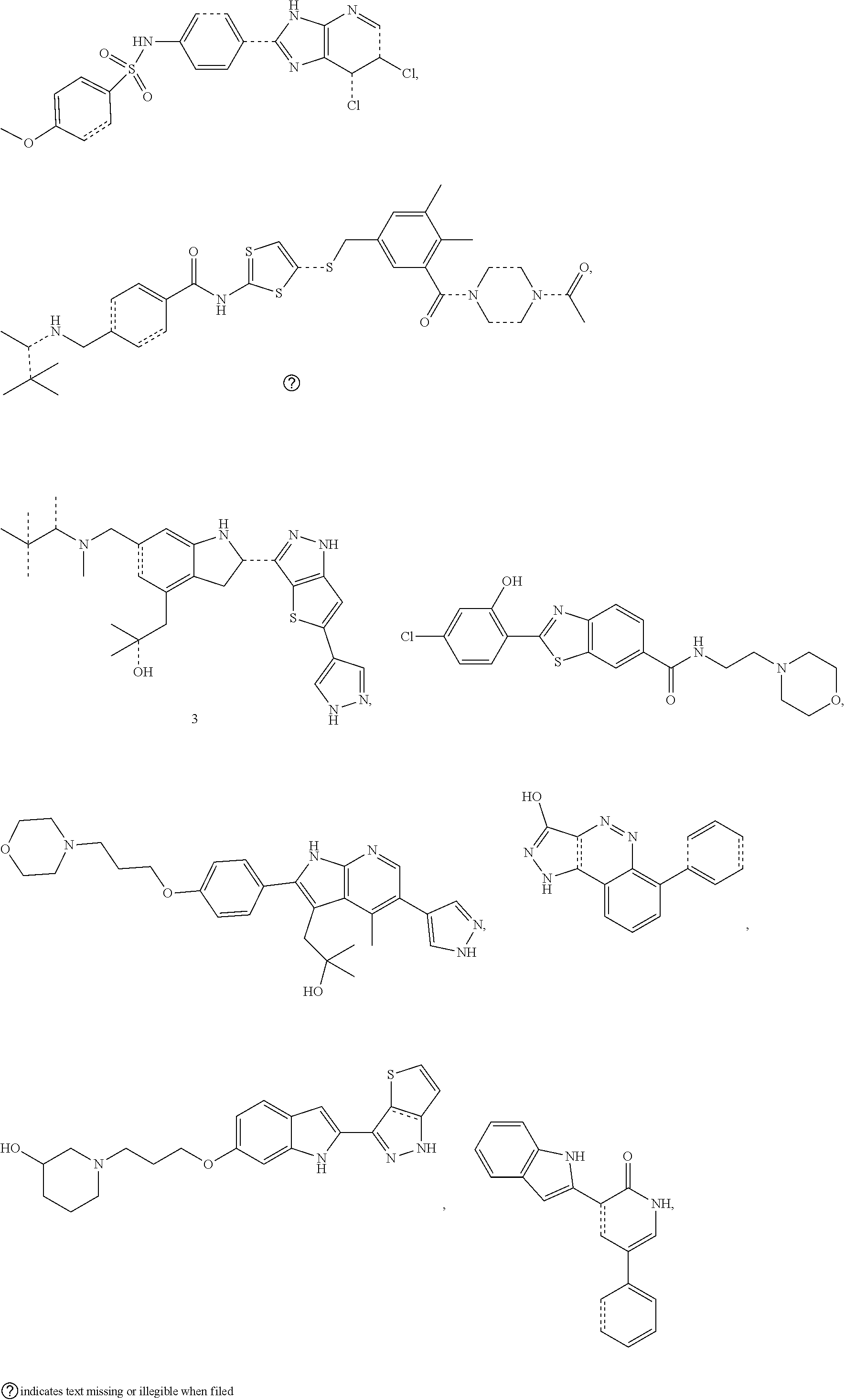

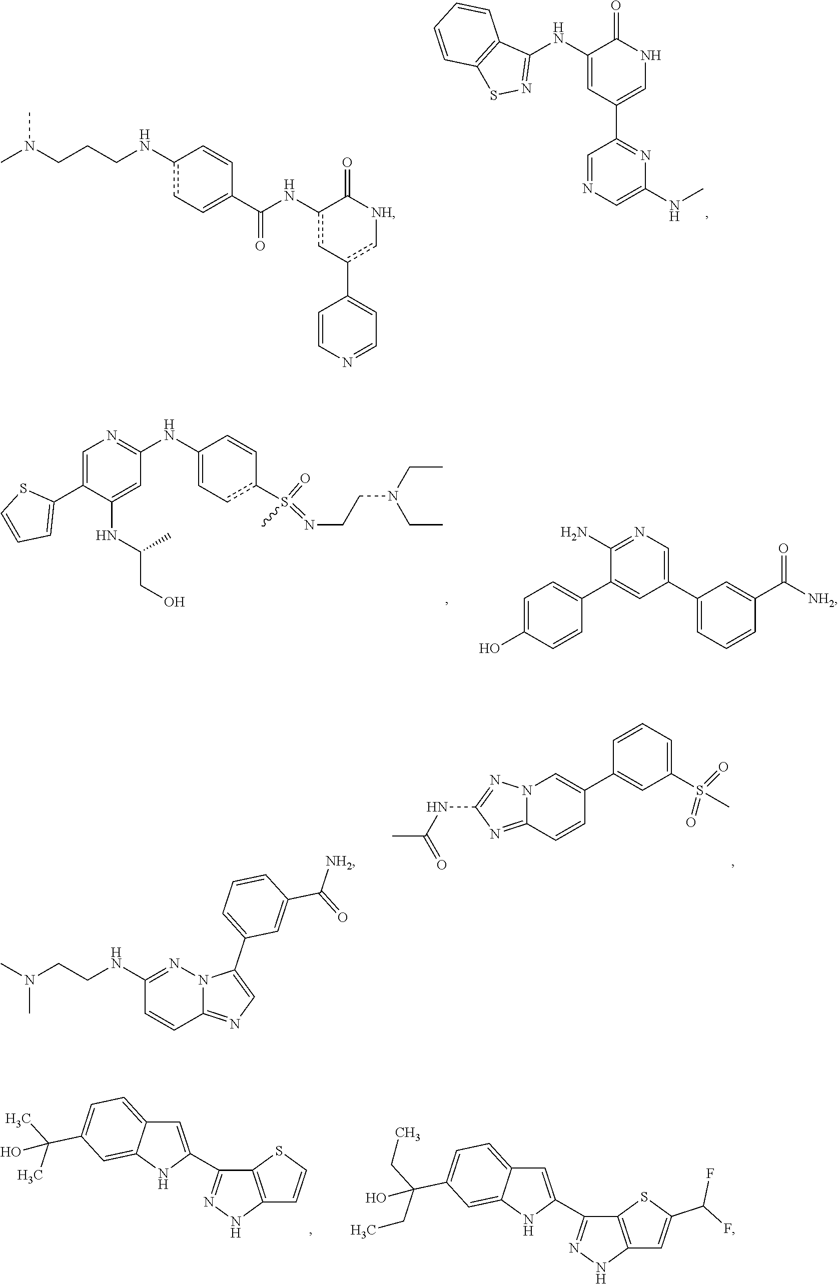

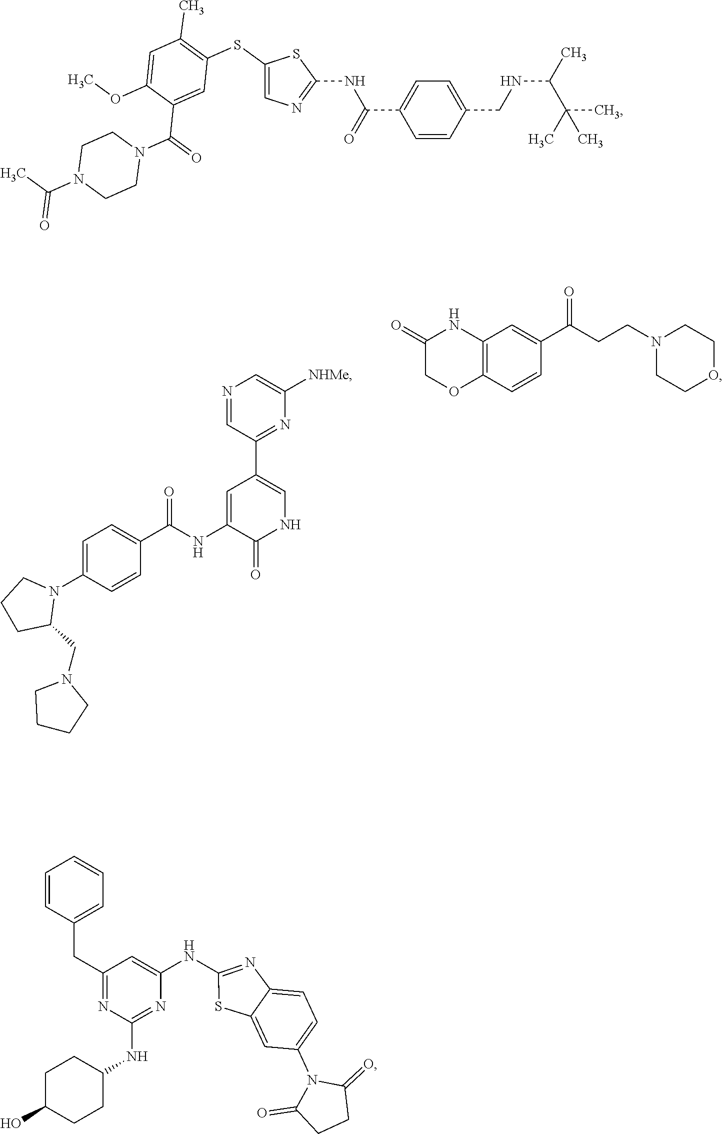

and combinations thereof. In an embodiment of the invention, the ITK inhibitor is selected from the group consisting of imatinib, dasatinib (BMS-354825), Sprycel [N-(2-chloro-6-methylphenyl)-2-(6-(4-(2-hydroxyethyl)-piperazin-1-yl)-2-meth-ylpyrimidin-4-ylamino)thiazole-5-carboxamide), ibrutinib ((1-{(3R)-3-[4-amino-3-(4-phenoxyphenyl)-1H-pyrazolo[3,4-d]pyrimidin-1-yl]piperidin-1-yl}prop-2-en-1-one), bosutinib, nilotinib, erlotinib, 1H-pyrazolo[4,3-c]cinnolin-3-ol, CTA056 (7-benzyl-1-(3-(piperidin-1-yl)propyl)-2-(4-(pyridin-4-yl)phenyl)-1H-imidazo[4,5-g]quinoxalin-6(5H)-one), Compound 10 (Boehringer Ingelheim from Moriarty, et al., Bioorg Med Chem Lett, 18:5537-40 (2008)), Compound 19 (Boehringer Ingelheim from Moriarty, et al., Bioorg Med Chem Lett., 18:5537-40 (2008)), Compound 27 (Boehringer Ingelheim from Moriarty, et al., Bioorg Med Chem Lett., 18:5537-40 (2008)), Compound 26 (Boehringer Ingelheim from Winters, et al., Bioorg Med Chem Lett., 18:5541-4 (2008)), Compound 37 (Boehringer Ingelheim from Cook, et al., Bioorg Med Chem Lett., 19:773-7 (2009)), Compound 41 (Boehringer Ingelheim from Cook, et al., Bioorg Med Chem Lett., 19:773-7 (2009)), Compound 48 (Boehringer Ingelheim from Cook, et al., Bioorg Med Chem Lett., 19:773-7 (2009)), Compound 51 (Boehringer Ingelheim from Cook, et al., Bioorg Med Chem Lett., 19:773-7 (2009)), Compound 10n (Boehringer Ingelheim from Riethe, et al., Bioorg Med Chem Lett., 19:1588-91 (2009)), Compound 10o (Boehringer Ingelheim from Riethe, et al., Bioorg Med Chem Lett., 19:1588-91 (2009)), Compound 7v (Vertex from Charrier, et al., J Med Chem., 54:2341-50 (2011)), Compound 7w (Vertex from Charrier, et al., J Med Chem., 54:2341-50 (2011)), Compound 7x (Vertex from Charrier, et al., J Med Chem., 54:2341-50 (2011)), Compound 7y (Vertex from Charrier, et al., J Med Chem., 54:2341-50 (2011)), Compound 44 (Bayer Schering Pharma from vonBonin, et al., Exp Dermatol., 20:41-7 (2011)), Compound 13 (Nycomed from Velankar, et al., Bioorg Med Chem., 18:4547-59 (2010)), Compound 24 (Nycomed from Velankar, et al., Bioorg Med Chem., 18:4547-59 (2010)), Compound 34 (Nycomed from Velankar, et al., Bioorg Med Chem., 18:4547-59 (2010)), Compound 10o (Nycomed from Herdemann, et al., Bioorg Med Chem Lett., 21:1852-6 (2011)), Compound 3 (Sanofi US from McLean, et al., Bioorg Med Chem Lett., 22:3296-300 (2012)), Compound 7 (Sanofi US from McLean, et al., Bioorg Med Chem Lett., 22:3296-300 (2012), and/or or other kinase inhibitors, tyrosine kinase inhibitors, or serine/threonine kinase inhibitors known in the art, as well as any combinations thereof.

In any of the foregoing embodiments, pre-treatment regimens comprising ibrutinib (commercially available as IMBRUVICA, and which has the chemical name 1-[(3R)-3-[4-amino-3-(4-phenoxyphenyl)-1H-pyrazolo[3,4-d]pyrimidin-1-yl]-1-piperidinyl]-2-propen-1-one) may include orally administering one 140 mg capsule q.d., orally administering two 140 mg capsules q.d., orally administering three 140 mg capsules q.d., or orally administering four 140 mg capsules q.d., for a duration of about one day, two days, three days, four days, five days, six days, seven days, eight days, nine days, ten days, eleven days, twelve days, two weeks, three weeks, one month, two months, three months, four months, five months, or six months. In the foregoing embodiments, pre-treatment regimens comprising ibrutinib may also comprise orally administering an ibrutinib dose selected from the group consisting of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, and 500 mg, wherein the administering occurs once daily, twice daily, three times daily, or four times daily, and wherein the duration of administration is selected from the group consisting of about one day, two days, three days, four days, five days, six days, seven days, eight days, nine days, ten days, eleven days, twelve days, two weeks, three weeks, one month, two months, three months, four months, five months, and six months.

In any of the foregoing embodiments, the cancer to be treated is a hematological malignancy selected from the group consisting of acute myeloid leukemia (AML), mantle cell lymphoma (MCL), follicular lymphoma (FL), diffuse large B cell lymphoma (DLBCL), activated B cell (ABC) DLBCL, germinal center B cell (GCB) DLBCL, chronic lymphocytic leukemia (CLL), CLL with Richter's transformation (or Richter's syndrome), small lymphocytic leukemia (SLL), non-Hodgkin's lymphoma (NHL), Hodgkin's lymphoma, relapsed and/or refractory Hodgkin's lymphoma, B cell acute lymphoblastic leukemia (B-ALL), mature B-ALL, Burkitt's lymphoma, Waldenström's macroglobulinemia (WM), multiple myeloma, myelodysplatic syndromes, myelofibrosis, chronic myelocytic leukemia, follicle center lymphoma, indolent NHL, human immunodeficiency virus (HIV) associated B cell lymphoma, and Epstein-Barr virus (EBV) associated B cell lymphoma.

The invention provides any of the foregoing embodiments modified as applicable such that the cancer to be treated is either resistant or refractory to treatment with an ITK inhibitor, such as ibrutinib, or has relapsed following a response to treatment with an ITK inhibitor, such as ibrutinib.

Efficacy of the methods and compositions described herein in treating, preventing and/or managing the indicated diseases or disorders can be tested using various animal models known in the art.

Non-Myeloablative Lymphodepletion with Chemotherapy

In an embodiment, the invention provides a method of treating a cancer with a population of TILs, wherein a patient is pre-treated with non-myeloablative chemotherapy prior to an infusion of TILs according to the present disclosure. In an embodiment, the non-myeloablative chemotherapy is one or more chemotherapeutic agents. In an embodiment, the non-myeloablative chemotherapy is cyclophosphamide 60 mg/kg/d for 2 days (days 27 and 26 prior to TIL infusion) and fludarabine 25 mg/m²/d for 5 days (days 27 to 23 prior to TIL infusion). In an embodiment, after non-myeloablative chemotherapy and TIL infusion (at day 0) according to the present disclosure, the patient receives an intravenous infusion of IL-2 intravenously at 720,000 IU/kg every 8 hours to physiologic tolerance.

Experimental findings indicate that lymphodepletion prior to adoptive transfer of tumor-specific T lymphocytes plays a key role in enhancing treatment efficacy by eliminating regulatory T cells and competing elements of the immune system (“cytokine sinks”). Accordingly, some embodiments of the invention utilize a lymphodepletion step (sometimes also referred to as “immunosuppressive conditioning”) on the patient prior to the introduction of the TILs of the invention.

In general, lymphodepletion is achieved using administration of fludarabine or cyclophosphamide (the active form being referred to as mafosfamide) and combinations thereof. Such methods are described in Gassner, et al., Cancer Immunol. Immunother. 2011, 60, 75-85, Muranski, et al., Nat. Clin. Pract. Oncol., 2006, 3, 668-681, Dudley, et al., J. Clin. Oncol. 2008, 26, 5233-5239, and Dudley, et al., J. Clin. Oncol. 2005, 23, 2346-2357, all of which are incorporated by reference herein in their entireties.

In some embodiments, the fludarabine is administered at a concentration of 0.5 μg/mL-10 μg/mL fludarabine. In some embodiments, the fludarabine is administered at a concentration of 1 μg/mL fludarabine. In some embodiments, the fludarabine treatment is administered for 1 day, 2 days, 3 days, 4 days, 5 days, 6 days, or 7 days or more. In some embodiments, the fludarabine is administered at a dosage of 10 mg/kg/day, 15 mg/kg/day, 20 mg/kg/day, 25 mg/kg/day, 30 mg/kg/day, 35 mg/kg/day, 40 mg/kg/day, or 45 mg/kg/day. In some embodiments, the fludarabine treatment is administered for 2-7 days at 35 mg/kg/day. In some embodiments, the fludarabine treatment is administered for 4-5 days at 35 mg/kg/day. In some embodiments, the fludarabine treatment is administered for 4-5 days at 25 mg/kg/day.

In some embodiments, the mafosfamide, the active form of cyclophosphamide, is obtained at a concentration of 0.5 μg/mL-10 μg/mL by administration of cyclophosphamide. In some embodiments, mafosfamide, the active form of cyclophosphamide, is obtained at a concentration of 1 μg/mL by administration of cyclophosphamide. In some embodiments, the cyclophosphamide treatment is administered for 1 day, 2 days, 3 days, 4 days, 5 days, 6 days, or 7 days or more. In some embodiments, the cyclophosphamide is administered at a dosage of 100 mg/m²/day, 150 mg/m²/day, 175 mg/m²/day 200 mg/m²/day, 225 mg/m²/day, 250 mg/m²/day, 275 mg/m²/day, or 300 mg/m²/day. In some embodiments, the cyclophosphamide is administered intravenously (i.e., i.v.) In some embodiments, the cyclophosphamide treatment is administered for 2-7 days at 35 mg/kg/day. In some embodiments, the cyclophosphamide treatment is administered for 4-5 days at 250 mg/m²/day i.v. In some embodiments, the cyclophosphamide treatment is administered for 4 days at 250 mg/m²/day i.v.

In some embodiments, lymphodepletion is performed by administering the fludarabine and the cyclophosphamide are together to a patient. In some embodiments, fludarabine is administered at 25 mg/m²/day i.v. and cyclophosphamide is administered at 250 mg/m²/day i.v. over 4 days.

In an embodiment, the lymphodepletion is performed by administration of cyclophosphamide at a dose of 60 mg/m²/day for two days followed by administration of fludarabine at a dose of 25 mg/m²/day for five days. Several methods of expanding TILs obtained from bone marrow or peripheral blood are described herein. In an embodiment of the invention, the lymphodepletion is performed by administration of cyclophosphamide at a dose of 60 mg/m²/day for two days followed by administration of fludarabine at a dose of 25 mg/m²/day for five days. Several methods of expanding TILs obtained from bone marrow or peripheral blood are described herein.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1—Selecting and Expanding PBLs from PBMCs Obtained from CLL Patients

PBMCs are collected from patients (optionally pretreated with an ITK inhibitor such as ibrutinib) and either frozen prior to use or used fresh. Enough volume of peripheral blood is collected to yield at least about 400,000,000 (400×10⁶) PBMCs for starting material in the method of the present invention. On Day 0 of the method, IL-2 at 6×10⁶ IU/mL is either prepared fresh or thawed, and stored at 4° C. or on ice until ready to use. 200 mL of CM2 medium is prepared by combining 100 mL of CM1 medium (containing GlutaMAX®), then diluting it with 100 mL (1:1) with AIM-V to make CM2. The CM2 is protected from light, and sealed tightly when not in use.

All of the following steps are performed under sterile cell culture conditions. An aliquot of 50 mL of CM2 is warmed in a 50 mL conical tube in a 37° C. water bath for use in thawing and/or washing a frozen PBMC sample. If a frozen PBMC sample is used, the sample is removed from freezer storage and kept on dry ice until ready to thaw. When ready to thaw the PBMC cryovial, 5 mL of CM2 medium is placed in a sterile 50 mL conical tube. The PBMC sample cryovial is placed in a 37° C. water bath until only a few ice crystals remain. Warmed CM2 medium is added, dropwise, to the sample vial in a 1:1 volume ratio of sample:medium (about 1 mL). The entire contents is removed from the cryovial and transferred to the remaining CM2 medium in the 50 mL conical tube. An additional 1-2 mL of CM2 medium is used to rinse the cryovial and the entire contents of the cryovial is removed and transferred to the 50 mL conical tube. The volume in the conical tube is then adjusted with additional CM2 medium to 15 mL and swirled gently to rinse the cells. The conical tube is then centrifuged at 400 g for 5 minutes at room temperature in order to collect the cell pellet.

The supernatant is removed from the pellet, the conical tube is capped, and then the cell pellet is disrupted by, for example, scraping the tube along a rough surface. About 1 mL of CM2 medium is added to the cell pellet, and the pellet and medium are aspirated up and down 5-10 times with a pipette to break up the cell pellet. An additional 3-5 mL of CM2 medium is added to the tube and mixed via pipette to suspend the cells. At this point, the volume of the cell suspension is recorded. Remove 100 μL of the cell suspension from the tube for cell counting with an automatic cell counter, for example, a Nexcelom Cellometer K2. The number of live cells in the sample is determined and recorded.

Reserve a minimum of 5×10⁶ cells for phenotyping and other characterization experiments. Spin the reserved cells at 400 g for 5 minutes at room temperature to collect the cell pellet. Resuspend the cell pellet in freezing medium (sterile, heat-inactivated FBS containing 20% DMSO). Freeze one or two aliquots of the reserved cells in freezing medium, each aliquot consisting of 2-5×10⁶ cells in 1 mL of freezing medium in a cryovial, and slow-freeze the aliquots in a cell freezer (Mr. Frosty) in a −80° C. freezer. Transfer to liquid nitrogen storage after a minimum of 24 hours at −80° C.

For the following steps, use pre-cooled solutions, work quickly, and keep the cells cold. The next step is to purify the T-cell fraction of the PBMC sample. This is completed using a Pan T-cell Isolation Kit (Miltenyi, catalog #130-096-535). Prepare the cells for purification by washing the cells with a sterile-filtered wash buffer containing PBS, 0.5% BSA, and 2 mM EDTA at pH 7.2. The PBMC sample is centrifuged at 400 g for 5 minutes to collect the cell pellet. The supernatant is aspirated off and the cell pellet is resuspended in 40 uL of wash buffer for every 10⁷ cells. Add 10 uL of Pan T Cell Biotin-Antibody Cocktail for every 10⁷ cells. Mix well and incubate for 5 minutes in refrigerator or on ice. Add 30 uL of wash buffer for every 10⁷ cells. Add 20 uL of Pan T-cell MicroBead Cocktail for every 10⁷ cells. Mix well and incubate for 10 minutes in refrigerator or on ice. Prepare an LS column and magnetically separate cells from the microbeads. The LS column is placed in the QuadroMACS magnetic field. The LS column is washed with 3 mL of cold wash buffer, and the wash is collected and discarded. The cell suspension is applied to the column and the flow-through (unlabeled cells) is collected. This flow-through is the enriched T-cell fraction (PBLs). Wash the column with 3 mL of wash buffer and collect the flow-through in the same tube as the initial flow-through. Cap the tube and place on ice. This is the T-cell fraction, or PBLs. Remove the LS column from the magnetic field, wash the column with 5 mL of wash buffer, and collect the non-T-cell fraction (magnetically labeled cells) into another tube. Centrifuge both fractions at 400 g for 5 minutes to collect the cell pellets. Supernatants are aspirated from both samples, disrupt the pellet, and resuspend the cells in 1 mL of CM2 medium supplemented with 3000 IU/mL IL-2 to each pellet, and pipette up and down 5-10 times to break up the pellets. Add 1-2 mL of CM2 to each sample, and mix each sample well, and store in tissue culture incubator for next steps. Remove about a 50 uL aliquot from each sample, count cells, and record count and viability.

The T-cells (PBLs) are then cultured with Dynabeads™ Human T-Expander CD3/CD28. A stock vial of Dynabeads is vortexed for 30 seconds at medium speed. A required aliquot of beads is removed from the stock vial into a sterile 1.5 mL microtube. The beads are washed with bead wash solution by adding 1 mL of bead wash to the 1.5 mL microtube containing the beads. Mix gently. Place the tube onto the DynaMag™-2 magnet and let sit for 30 minutes while beads draw toward the magnet. Aspirate the wash solution off the beads and remove tube from the magnet. 1 mL of CM2 medium supplemented with 3000 IU/mL IL-2 is added to the beads. The entire contents of the microtube is transferred to a 15 or 50 mL conical tube. Bring the beads to a final concentration of about 500,000/mL using CM2 medium with IL-2.

The T-cells (PBLs) and beads are cultured together as follows. On day 0: In a G-Rex 24 well plate, in a total of 7 mL per well, add 500,000 T-cells, 500,000 CD3/CD28 Dynabeads, and CM2 supplemented with IL-2. The G-Rex plate is placed into a humidified 37° C., 5% CO₂ incubator until the next step in the process (on Day 4). Remaining cells are frozen in CS10 cryopreservation medium using a Mr. Frosty cell freezer. The non-T-cell fraction of cells are frozen in CS10 cryopreservation medium using a Mr. Frosty cell freezer. On day 4, medium is exchanged. Half of the medium (about 3.5 mL) is removed from each well of the G-rex plate. A sufficient volume (about 3.5 mL) of CM4 medium supplemented with 3000 IU/mL IL-2 warmed to 37° C. is added to replace the medium removed from each sample well. The G-rex plate is returned to the incubator.

On day 7, cells are prepared for expansion by REP. The G-rex plate is removed from the incubator and half of medium is removed from each well and discarded. The cells are resuspended in the remaining medium and transferred to a 15 mL conical tube. The wells are washed with 1 mL each of CM4 supplemented with 3000 IU/mL IL-2 warmed to 37° C. and the wash medium is transferred to the same 15 mL tube with the cells. A representative sample of cells is removed and counted using an automated cell counter. If there are less than 1×10⁶ live cells, the Dynabead expansion process at Day 0 is repeated. The remainder of the cells are frozen for back-up expansion or for phenotyping and other characterization studies. If there are 1×10⁶ live cells or more, the REP expansion is set up in replicate according to the protocol from Day 0. Alternatively, with enough cells, the expansion may be set up in a G-rex 10M culture flask using 10-15×10⁶ PBLs per flask and a 1:1 ratio of Dynabeads:PBLs in a final volume of 100 mL/well of CM4 medium supplemented with 3000 IU/mL IL-2. The plate and/or flask is returned to the incubator. Excess PBLs may be aliquotted and slow-frozen in a Mr. Frosty cell freezer in a −80° C. freezer, and the transferred to liquid nitrogen storage after a minimum of 24 hours at −80° C. These PBLs may be used as back-up samples for expansion or for phenotyping or other characterization studies.

On Day 11, the medium is exchanged. Half of the medium is removed from either each well of the G-rex plate or the flask and replaced with the same amount of fresh CM4 medium supplemented with 3000 IU/mL IL-2 at 37° C.

On Day 14, the PBLs are harvested. If the G-rex plate is used, about half of the medium is removed from each well of the plate and discarded. The PBLs and beads are suspended in the remaining medium and transferred to a sterile 15 mL conical tube (Tube 1). The wells are washed with 1-2 mL of fresh AIM-V medium warmed to 37° C., and the wash is transferred to Tube 1. Tube 1 is capped and placed in the DynaMag™-15 Magnet for 1 minute to allow the beads to be drawn to the magnet. The cell suspension is transferred into a new 15 mL tube (Tube 2), and the beads are washed with 2 mL of fresh AIM-V at 37° C. Tube 1 is placed back in the magnet for an additional 1 minute, and the wash medium is then transferred to Tube 2. The wells may be combined if desired, after the final washing step. Remove a representative sample of cells and count, record count and viability. Tubes may be placed in the incubator while counting. Additional AIM-V medium may be added to the Tube 2 if cells appear very dense. If a flask is used, the volume in the flask should be reduced to about 10 mL. The contents of the flask is mixed and transferred to a 15 mL conical tube (Tube A). The flask is washed with 2 mL of the AIM-V medium as described above and the wash medium is also transferred to Tube A. Tube A is capped and placed in the DynaMag™-15 Magnet for 1 minute to allow the beads to be drawn to the magnet. The cell suspension is transferred into a new 15 mL tube (Tube B), and the beads are washed with 2 mL of fresh AIM-V at 37° C. Tube A is placed back in the magnet for an additional 1 minute, and the wash medium is then transferred to Tube B. The wells may be combined if desired, after the final washing step. Remove a representative sample of cells and count, record count and viability. Tubes may be placed in the incubator while counting. Additional AIM-V medium may be added to the Tube B if cells appear very dense. Cells may be used fresh or frozen in CS10 preservation medium at desired concentrations.

### Example 2—Alternative Method for Selecting and Expanding PBLs from PBMCs Obtained from CLL Patients

For the expansion of PBLs from PBMCs obtained from CLL patients or patients with other diseases described herein, including CLL patients having previously received ibrutinb or an ITK inhibitor or with ibrutinib-relapsed or refractory CLL, the following procedure may be used. All steps require the use of sterile technique in a biological safety cabinet (BSC) or similar enclosure.

On day 0, prepare 6×10⁶ IU/mL IL-2. If aliquots are available, thaw a fresh aliquot and leave it at 4° C. in refrigerator or on ice until ready to use. Prepare small volume (e.g. 200 mL) of CM2 media. First prepare 100 mL of CM1 media, substituting GlutaMAX for glutamine in the procedure, then dilute it 1:1 with AIM V to make CM2. While performing this experiment, keep the CM2 warm in a 37° C. water bath, protected from light, with cap closed tightly. When it is being used in the hood, do not leave the cap off or loose. In a 37° C. water bath, warm an aliquot of CM2 (without IL-2) in a 50 mL conical tube to use for thawing and washing CLL sample. Remove the cryovial containing the CLL PBMC sample from LN2 freezer storage, keeping sample on dry ice until ready to thaw, or use fresh CLL PBMCs. Just prior to beginning thaw, place warmed media aliquot into BSC. Add 5 mL of media into a fresh, sterile, labeled 50 mL conical tube. Thaw sample by placing the cryovial in 37° C. water bath until only a few ice crystals remain in the cryovial. Transfer cryovial containing thawed samples to the BSC. Using a sterile transfer pipet, add an equal volume of warmed media dropwise to CLL sample cryovial (˜1 mL). Using the same transfer pipet, remove the sample from the cryovial and add it dropwise to the prepared 50 mL conical tube. Rinse tube with an additional 1-2 mL of CM2 and transfer that to the 50 mL conical tube. Bring the volume to 15 mL, swirl sample gently to rinse cells well, then spin sample in high speed centrifuge to collect cell pellet, at 400×g for 5 min at room temperature. Return sample to BSC and aspirate off supernatant from pellet, being careful not to disturb cell pellet. Cap tube and scrape it along a rough surface (such as a tube rack) to help break up cell pellet. Using a 1 mL pipettor and tip, add 1 mL of fresh CM2 to the cell pellet and gently aspirate the cells up-and-down 5-10 times to break up cell pellet. Add an additional 3-5 mL of CM2 to cell suspension; pipet up-and-down several times to mix sample well. Record volume of cell suspension. Remove a representative volume of cell suspension from the tube for counting (e.g., 100 Using an automatic cell counter, such as Nexcelom Cellometer K2, count cells using appropriate procedure. Determine the total number of live cells in the sample. Reserve a minimum of 5×10⁶ cells for phenotyping and other experiments. Spin reserved sample at 400×g for 5 min at room temperature to collect pellet. Freeze one or two aliquots of the reserved sample in freezing medium (sterile, heat-inactivated fetal bovine serum containing 20% DMSO). Slow-freeze cell sample in a Mr. Frosty cell freezer placed in a −80° C. freezer. Transfer to LN2 storage after a minimum of 24 hours at −80° C.

For the proceeding separation steps, work quickly, keeping the cells cold; use pre-cooled solutions. Purify the T-cell fraction of the CLL sample using Pan T-cell Isolation Kit (Miltenyi: Catalogue #130-096-535). Prepare wash buffer prior to beginning procedure. Wash buffer: phosphate-buffered saline, pH 7.2, containing 0.5% bovine serum albumin and 2 mM EDTA; read pH and adjust if necessary; sterile filter; store at 4° C. Spin sample at 400×g for 5 min to collect cell pellet. Aspirate off media supernatant and resuspend the cell pellet in 40 of wash buffer for every 10⁷ total cells. Add 10 μL of Pan T Cell Biotin-Antibody Cocktail for every 10⁷ total cells. Mix sample well and incubate in the refrigerator or on ice for 5 min. Add 30 μL of wash buffer to sample for every 10⁷ total cells. Add 20 μL of Pan T Cell MicroBead Cocktail for every 10⁷ total cells. Mix well and incubate for 10 min in the refrigerator or on ice. Proceed to magnetic cell separation. Use LS column and QuadroMACS magnet for this procedure. Each LS column has a maximum capacity of 2×10⁹ total cells. Prepare LS column for use; always wait until column reservoir is empty before proceeding to the next step. Place LS column in magnetic field of QuadroMACS magnet. Rinse LS column with 3 mL of prepared, cold wash buffer. Collect wash into a 15 mL conical tube. Discard wash. Place fresh tube labeled “T cell fraction” under LS column. Apply cell suspension onto the column. Collect flow-through containing the unlabeled cells—this is the enriched T-cell fraction. Wash column with 3 mL of wash buffer. Collect the unlabeled cells that wash through the column into the same 15 mL conical “T cell fraction” tube. Cap tube and place on ice. Remove LS column from QuadroMACS magnet and place it onto a fresh 15-ml conical tube labeled “non-T cell fraction.” Pipet 5 mL of wash buffer onto the column and immediately flush out the magnetically labeled non-T cells by firmly pushing plunger (provided with the LS column) into the column. Place both “T-cell fraction” and “Non-T cell fraction” tubes into centrifuge and spin at 400×g for 5 min to collect cell pellet. Aspirate off supernatant from both samples, cap tubes, and resuspend each pellet by scraping tube against a rough surface. Using a 1 mL pipettor and tips, add 1 mL of CM2 medium supplemented with 3000 IU/ml IL-2 to each pellet. Resuspend each pellet by gently pipetting up-and-down 5-10 times to break pellets up further. Add 1-2 mL of fresh medium to each sample, mixing each sample well. Remove a small representative aliquot from each sample (e.g., 50 μL). Place cell samples into tissue culture incubator, loosening cap. Count cells; record counts and viability. Prepare a small amount of Dynabeads™ Human T-Expander CD3/CD28 for use. Vortex stock vial of CD3/CD28 Dynabeads for 30 sec at medium speed on a vortex mixer. Remove required aliquot of beads from stock vial to a sterile 1.5 mL microtube Wash beads with bead wash solution by adding 1 mL of wash to the 1.5 mL microtube containing the beads. Tap tube to mix sample. Place tube containing beads onto DynaMag™-2 magnet and let tube sit for 30 sec while beads are drawn to magnet. Aspirate off wash solution from the side of the microtube opposite the DynaMag magnet. Remove microtube from magnet and place in a tube rack. Using a 1 mL pipettor and tip, add 1 ml of CM2 supplemented with IL-2 to the beads. Transfer the bead solution to a fresh 15 mL (or 50 mL) conical tube labeled “beads, 500,000/mL.” Bring beads to a final volume that will give a concentration of 500,000 beads/ml (e.g., 10×10⁶ beads brought to a final volume of 20 mL). Set up cell culture as follows, a minimum of 1 well per sample. More wells can be set up if there are enough cells. In a G-Rex 24-well plate, in a total of 7 mL per well, add 500,000 T cells, 500,000 CD3/CD28 Dynabeads (1 mL of 500,000 beads/mL suspension), and CM2 supplemented with 3000 IU/ml IL-2. Place G-Rex 24-well plate into humidified 37° C., 5% CO₂ incubator. If there are enough cells, retain a small portion of the T-cell fraction for repeat of REP, or for other experiments, freezing the sample in CS10 cryopreservation medium using a Mr. Frosty cell freezer. Count the non-T cell fraction of cells and freeze them in CS10 cryopreservation medium using a Mr. Frosty cell freezer.

On day 4, media exchange is performed as follows. Prepare a sufficient volume of CM4 (supplemented with 3000 IU/mL IL-2) to replace half the media from the sample wells and warm it to 37° C. in water bath. Remove G-Rex 24-well plate from incubator to BSC. Remove half the volume of media from each well (3.5 mL). Add equivalent volume (3.5 mL) of fresh media to each well. Return G-Rex 24-well plate to humidified, 37° C., 5% CO₂ incubator.

On day 7, expansion using REP is performed as follows. Prepare a small volume of CM4 (supplemented with 3000 IU/mL IL-2) to perform washes of culture wells. Keep warm in 37° C. water bath. Remove G-Rex 24-well plate from the incubator to BSC. Remove half the volume of media from each well and discard. Resuspend remaining cells and transfer to a labeled, sterile 15 mL conical tube. Wash well with 1 mL of prepared CM4 and transfer wash solution to the same 15 mL conical tube. Retain the G-Rex 24-well plate in the tissue culture hood—unused wells of the same plate can be used for the expansion of the PBL sample. Remove a representative volume of cells and count using automated cell counter. Determine number of wells or culture vessels required to expand PBL. If ≤1×10⁶ total live cells: Set up expansion in one well of a G-Rex 24-well plate using 500,000 PBL and a 1:1 ratio of Dynabeads™ Human T-Expander CD3/CD28 prepared as in Step 9.16 in 7 mL CM4 (supplemented with 3000 IU/mL IL-2). Freeze remainder of cells for back-up expansion or for phenotyping and other subsidiary procedures. If 1×10⁶ or more total live cells: set up expansion in replicate wells in a G-Rex 24-well plate using 500,000 PBL per well and a 1:1 ratio of Dynabeads™ Human T-Expander CD3/CD28 prepared as in Step 9.16 in final volume of 7 ml/well CM4 (supplemented with 3000 IU/mL IL-2). Alternately, with excess sample, set up expansion in a G-Rex10M culture flask using 10-15×10⁶ PBL per flask and a 1:1 ratio of Dynabeads™ Human T-Expander CD3/CD28 prepared as in Step 9.16 in a final volume of 100 ml/well CM4 (supplemented with 3000 IU/mL IL-2). Slow-freeze excess Day 7 PBL samples in labeled cryovials placed in a Mr. Frosty cell freezer at −80° C. Transfer to LN2 storage after a minimum 24 hours at −80° C. These samples can be used as back-up samples for expansion or for phenotyping and other subsidiary procedures. (Recommended minimum number of cells to retain on Day 7: 2×10⁶ to 5×10⁶.) Place culture plates or flasks into humidified, 37° C., 5% CO₂ incubator.

On day 11, media exchange is performed as follows. Prepare a sufficient volume of CM4 (supplemented with 3000 IU/mL IL-2) to replace half of the volume in each culture well or vessel and keep it warm in a 37° C. water bath. Remove the culture vessels from the incubator to the BSC. Remove half the media from each well or flask and discard. Add equivalent volume to each culture well or flask. Return culture vessels to humidified, 37° C., 5% CO₂ incubator.

On day 14, REP harvest is performed as follows. Warm a small volume of AIM V media in a 37° C. water bath to use for washes in the following steps. Transfer to BSC when ready to harvest samples. Remove the culture vessels from the incubator to the BSC. If culture is in G-Rex 24-well plate, remove about half of the volume from each well and discard. For larger cultures, proceed to the “REP is complete” step in the next paragraph. Mix sample with serological pipet and transfer cells to labeled, sterile 15 mL conical tube. Wash well with 1-2 mL of fresh, warmed media. Cap 15 mL conical tube and place in DynaMag™-15 Magnet. Allow sample to sit for 1 min in magnet to allow magnetic beads to be drawn to magnet. Using a 5 mL serological pipet, remove the cell suspension to a fresh, labeled 15 mL conical tube. Remove first 15-ml tube from magnet and wash the beads with a minimum of 2 ml of fresh AIM V. Place tube back on magnet and allow it to sit for 1 min. Using a 5 mL serological pipet, remove the wash media to the second labeled 15 mL conical tube. If more than one well per sample was prepared, all wells of the same condition can be combined after washing the beads. If culture is in G-Rex10M flask, reduce volume by aspiration to about 10 mL total. Mix sample using 10 mL serological pipet and transfer cells to labeled, sterile 15 mL conical tube. Wash flask with 2 mL of fresh, warmed media. Cap 15 mL conical tube and place in DynaMag™-15 Magnet. Allow sample to sit for 1 min in magnet to allow magnetic beads to be drawn to magnet. Using a 5 mL serological pipet, remove the cell suspension to a fresh, labeled 15 mL conical tube. Remove first 15 mL tube from magnet and wash the beads with a minimum of 2 mL of fresh AIM V. Place tube back on magnet and allow it to sit for 1 min. Using a 5 mL serological pipet, remove the wash media to the second labeled 15 mL conical tube. If more than one flask per sample was prepared, all can be combined after washing the beads. If cells appear to be extremely dense, extra pre-warmed AIM V media can be added to the culture. Remove a representative volume of cells and count using automated cell counter. Record cell number and viability. Place tubes containing cells into humidified, 37° C., 5% CO2 incubator, with cap loosened, while counting cells.

At this point, the REP is complete. Post-REP testing of PBL can be done on fresh or frozen samples. Freeze PBL samples in CS10 cryopreservation medium, or prepare as needed in alternative formulations for delivery to a patient. Lower concentrations of cells (e.g., 5×10⁶ cells/vial) can be used for phenotyping by flow cytometry and co-culture assays, so it is recommended to reserve 6-10 vials at low concentration, with the remainder at a higher concentration (30-50×10⁶ cells/vial). The foregoing procedure may be scaled, adjusted, or optimized, and adapted as needed for regulatory compliance (including to satisfy good manufacturing practices and International Conference on Harmonization guidance, as adapted by the U.S. Food and Drug Administration and other regulatory authorities), as will be apparent to the skilled artisan.

### Example 3—Full-Scale Manufacturing Process of PBL from Cryopreserved PBMCs of CLL Patients

This example illustrates an embodiment of a full scale manufacturing process for autologous PBL product for treatment of patients with CLL or other hematological malignancies. The experiments are performed on three cryopreserved PBMC samples obtained from different CLL patients who were treated with ibrutinib. All open manipulations of cell products take place within a Biosafety Cabinet in an ISO5 environment.

The materials in Table 3 are used in the process:

TABLE 3
Materials used in an exemplary embodiment
of a PBL manufacturing process.

| Material | Manufacturer/Vendor | Catalogue # |
|---|---|---|
| CTS Dynabeads | Life Technologies | 43500D |
| (CD3/CD28) | | |
| DNase-I GMP 4kU | Roche | 3724751103 |
| Human Serum | Octapharma | 68982-0643-01 |
| Albumin 25% | | |
| DPBS no Calcium no | Sigma or equivalent | D8537 |
| Magnesium | | |
| GRex 100MCS flasks | Wilson-Wolf | 81100-CS |
| Plasma-Lyte A | Baxter | JB2554 |
| Cryostor 10 | BioLife | 210102 |
| CliniMACS CD19 | Miltenyi | 130-019-301 |
| microbeads | | |
| CliniMacs DTS tubing | Miltenyi | 130-083-404 |

The equipment in Table 4 is used in the process:

TABLE 4
Equipment used in an exemplary embodiment
of a PBL manufacturing process.

| Equipment | Manufacturer/Vendor | Catalogue # |
|---|---|---|
| DynaMag-2 Magnet | ThermoFisher | 12321D |
| DynaMag-15 Magnet | ThermoFisher | 12301D |
| DynaMag-50 Magnet | ThermoFisher | 12302D |
| CliniMacs Plus | Miltenyi | 151-01 |
| Instrument | | |
| NC200 Nucleocounter | Chemometec | 900-0201 |

The starting material for the process described in this example is cryopreserved PBMCs that are obtained by Ficoll separation from CLL patient whole blood and cryopreserved at the collection site. Prior to enrichment, the percentage of CD3⁺ cells in the live population is determined using flow cytometry.

### Day 0 Procedure

Prepare 100 mL of wash/staining buffer to be used on Day 0 and bring to room temperature before use, using 95 mL of Sterile PBS, 4 mL of Human Serum Albumin (25%) for a final concentration of 1% human serum albumin, and 1 mL of DNAse I (1000 U/mL) for a final concentration of 10 U DNase I/mL. Prepare 500-2500 mL of CM2 and warm in 37° C. water bath for a minimum of 1 hour before use. Prepare IL-2 aliquots as needed, and add IL-2 (6×10⁶ IU/mL) to the CM2 for a final IL-2 concentration of 3000 IU/mL.

A wash sample is prepared as follows. Label a sterile 15 mL conical tube. Add about 10 mL of wash buffer to the labelled 15 mL conical tube. Thaw the cryopreserved PBMCs in a 37° C. waterbath for about 3 minutes, until there is almost no ice. Immediately transfer the thawed PBMCs into the labelled 15 mL conical tube and mix well by pipetting up and down. Rinse the original PBMC cryovial using about 1 mL of wash buffer and transfer the rinse to the labelled 15 mL conical tube. Mix well and remove a 200 μL sample for count and viability testing. Wash the cells via centrifugation at 400 g for 5 minutes at 24° C. (acceleration=9, deceleration=9). During centrifugation, mix the CTS Dynabeads by placing on the rocker for at least 5 minutes. Remove the cells from the centrifuge and transfer all the media into a clean 50 mL conical tube labelled “Wash-off”. Cap tubes and scrape them along a rough surface (such as a tube rack) to help break up the cell pellet.

Determine cell count and viability by performing a 1:10 dilution of the pre-wash sample in AIM-V media and using a standard cell count and viability protocol.

Perform T cell enrichment by positive selection of T cells using CTS CD3/C28 Dynabeads as follows. Calculate and record the number of CD3⁺ viable cells in the tube: Number of CD3⁺ viable cells=% CD3⁺ cells×TVC. Resuspend the cells using wash buffer, so the concentration of the viable CD3⁺ cells is 1×10⁷/mL after addition of the beads. The cell suspension final resuspension volume (μL) is determined as: Total # of viable CD3⁺ cells/1×10⁷)*1000. The volume of wash buffer to add (μL) to cells is calculated as: cell suspension final resuspension volume (μL)−500 (μL).

Calculate the required number and volume of the CTS Dynabeads: Number of required CTS Dynabeads=3*(Number of CD3⁺ viable cells). Required Volume of CTS Dynabeads (μL)=(Number of required CTS Dynabeads/4×10⁸)*1000. Vortex the CTS DynaBeads (on low to medium) for 30 seconds to 1 minute and visually ensure the dispersion of bead precipitates from the vial walls. Inside the BSC, transfer the required volume of CTS Dynabeads to a microtube. Add 1 mL of wash buffer to the microtube. Place the tube in a DynaMag-2 magnet for 1 min. Discard the supernatant. Remove the tube from the magnet and resuspend the washed Dynabeads in 0.5 mL of wash buffer. Add the washed CTS DynaBeads (CD3/28) at 3 beads: 1 T-cell ratio by transferring the volume as calculated above to the cells in the 15 mL conical tube. Incubate the sample with the Dynabeads, in the 15 mL conical tube covered with foil, on a rocker (1-3 RPM end to end) at room temperature for 30 (+5) minutes. Bring the volume up to 10 mL using CM2 plus IL-2 and mix well using a pipettor. Place the tube on the DynaMag-15 for one to two minutes for positive selection of the bead-bound CD3⁺ cells. Carefully pipette off the cell suspension (negative portion) into a 50 mL conical tube labelled (no T cell fraction). Take the 15 mL tube, which contains the bead-bound cells, off the magnet and immediately add 10 mL of CM2 media with IL-2 (3000 IU/mL) and mix well by pipetting up and down to disperse the bead clumps. Place the tube on the Dynamag-15 for one to two minutes. Carefully pipette off the cell suspension (residual negative portion) into the 50 mL conical tube labeled (no T cell fraction). Take the 15 mL tube, that contains the bead-bound cells, off the magnet and immediately add 10 mL of CM2 media with IL-2 (3000 IU/mL) and mix well by pipetting up and down to disperse the bead clumps. Place the tube on the Dynamag-15 for one to two minutes. Carefully pipette off the cell suspension (residual negative portion) into the 50 mL conical tube labeled (no T cell fraction). Immediately add 10 mL of CM2 media with IL-2 (3000 IU/mL) to the 15 mL tube that contains the bead-bound cells, remove it from the magnet and mix well. Relabel the tube as (T cell fraction). Count negative fraction to determine positive fraction count: TVC positive fraction=TVC pre-wash−TVC negative fraction. Obtain about 1×10⁶ cells from the negative fractions for flow analysis (CD3/4/8/19/14) of the fresh sample. Use normal donor PBMCs for the FMOs and as a positive control. Cryopreserve the leftover negative portion (target cells) in CS10. Determine the required number of G-REX 100MCS using the following formula, rounding up to the nearest whole number: Number of G-REX 100MCS=TVC positive fraction/5×10⁶. Determine the volume of positive fraction to transfer to each flask based on Table 5.

TABLE 5
Volume of positive fraction to transfer to each flask.

| # of G-REX flasks | 1 | 2 | 3 | 4 | 5 |
|---|---|---|---|---|---|
| Volume of Bead-cell suspension to | 10 | 5 | 3.3 | 2.5 | 2 |
| transfer to each G-REX flask (mL) | | | | | |

Transfer about 360 mL of CM2 plus IL-2 to each the G-REX 100MCS via a peristaltic pump. Attach a transfer set with a 20 mL syringe to one of the short tubes of the first G-REX100MCS. Inside the hood, pull out the syringe plunger. Transfer the positive fraction from the 15 mL conical tube to the G-REX 100MCS through the 20 mL syringe bore using a 10 mL pipette. Using the same 10 mL pipette, add 10 mL to the 15 mL conical tube to rinse. Transfer the rinse to the G-REX 100MCS through the 20 mL syringe bore using the 10 mL pipette. Using the same pipette, repeat the rinse step two more times. Transfer 360 mL of CM2+IL-2 to each the G-REX 100MCS via a peristaltic pump. Place the flasks in the incubator at 37° C. and 5% CO₂.

### Day 4 Procedure

Prepare media as follows. In a 3000 mL transfer bag, prepare 600 mL of CM4 per G-REX 100MCS flask. Warm in 37° C. water bath for a minimum of 1 hour before use. Prepare IL-2 aliquots if needed. Add the IL-2 to the CM4 for a final IL-2 concentration of 3000 IU/mL. Add the CM4 plus IL-2 to the cells. Obtain the G-REX 100MCS from the incubator. Sterile weld the transfer bag containing the media to the G-REX 100MCS. Pump in the 600 mL of CM4 plus IL-2 from the transfer bag to each G-REX 100MCS. Place the G-REX 100MCS back in the incubator.

### Day 9 Procedure

Prepare 3 L of harvest media (referred to herein as “Harvest Media”) using Plasmalyte+1% HAS at room temperature. Harvest cells by sterile welding a 3000 mL waste bag to the red line of the first G-REX 100MCS. Sterile weld a 600 mL transfer bag, labelled “Harvest” to the white/blue line of the G-REX. Using the GatheREX pump, reduce the volume of the media to ˜ 1/10^{th} the original volume. Mix the cell suspension in the G-REX100MCS. Using the GatheREX pump, harvest the cells in the transfer bag labelled “Harvest”. Repeat with all G-REX flasks. Centrifuge the cells at 300 g for 15 minutes at 24° C. (acceleration=9, without brake). Remove the supernatant using a plasma expressor into a sterile welded waste bag. Resuspend the cells using “Harvest Media” for a final volume of about 100-120 mL. Label four sterile 50 mL tubes with “Harvest”. Using 60 mL syringes, transfer about 30 mL of harvest product from the “Harvest” bag to the 50 mL conical tubes labelled “Harvest”. Use a clean syringe with each draw. Place the conical in a Dynamag-50 for one to two minutes for bead removal. Using a 25 mL pipette, remove the cell suspension into another 50 mL conical tube labelled with “wash-1” and keep inside the BSC. Immediately add 10 mL of Plasmalyte A plus 1% HSA into the tubes labelled “Harvest”. Mix and return to the magnet. Place the 50 mL conical again on the DynaMag-50 for 1-2 minutes to rinse. Using a 10 mL pipette, remove the cell suspension into the 50 mL conical tube labelled “wash-1”. Place the 50 mL conical labelled “wash-1” on the DynaMag-50 for 1-2 minutes to remove residual beads. Using a 50 mL pipette, remove the cell suspension into another 50 mL conical tube labelled with “wash-2” and keep. Place the 50 mL conical labelled “wash-2” on the DynaMag-50 for 1-2 minutes for one final removal of residual beads. Using a 50 mL pipette, remove the cell suspension into a 600 mL transfer bag labelled “final” via an attached 60 mL syringe bore. Remove a sample for cell count and viability and for bead residual count. Add 1 vial of CD19+ microbeads. Mix beads and cells and incubate for 30 minutes at room temperature on an orbital shaker in dark (about 25 RPM). Add about 400 mL of “Harvest Media”. Centrifuge at 300×g for 15 minutes at 24° C. (acceleration=9, without brake). Remove the supernatant using a plasma expressor into a sterile welded waste bag. Resuspend the cell pellet in 150 mL of “Harvest Media”. Assemble the DTS tubing and “Harvest Media” to the CliniMACS Plus. Proceed with the automated separation using the CliniMACS Plus instrument. Collect flow-through. Filter flow-through via a 170 μm blood filter into a 600 mL bag labelled “PRE-LOVO”. Obtain samples for count and residual bead counts from the bag labelled “Pre-LOVO”. Attach the pre-LOVO bag to the LOVO Cell Harvester (Fresenius Kabi) and follow standard procedures for final formulation and cryopreservation.

Exemplary acceptance criteria for PBL product are given in Table 6.

TABLE 6
Exemplary acceptance criteria for PBL product.

| Method | Test | Acceptance Criteria |
|---|---|---|
| Cell Counter | Total Viable Count | 1e9-150e9 |
| | % Viability | ≥70% |
| Flow cytometry | % of Total Viable cells | ≥90% |
| | CD3⁺CD45⁺ | |
| Interferon | Interferon Gamma | 500 pg/mL/5e5 viable cells |
| Gamma ELISA | produced | beads for 24 hours - |
| Assay | | Unstimulated for 24 hours) |

### Example 3A—Full-Scale Manufacturing Process of PBL from Cryopreserved PBMCs of CLL Patients with CliniMACS B-Cell Depletion on Day 0 or Day 9 of a 9-Day Expansion Process

This example illustrates an embodiment of a full scale manufacturing process for autologous PBL product for treatment of patients with CLL or other hematological malignancies, with depletion of B cells on Day 0 or Day 9 of the process. The experiments were performed on cryopreserved PBMC samples obtained from different CLL patients who were previously treated with ibrutinib. All open manipulations of cell products take place within a Biosafety Cabinet in an ISO5 environment.

The materials in Table 7 were used in the process:

TABLE 7
Materials used in an exemplary embodiment
of a PBL manufacturing process.

| Material | Manufacturer/Vendor | Catalogue # |
|---|---|---|
| CTS Dynabeads | Life Technologies | 43500D |
| (CD3/CD28) | | |
| DNase-I GMP 4kU | Roche | 3724751103 |
| Human Serum | Octapharma | 68982-0643-01 |
| Albumin 25% | | |
| DPBS no Calcium | Sigma or equivalent | D8537 |
| no Magnesium | | |
| GRex 100MCS flasks | Wilson-Wolf | 81100-CS |
| Plasma-Lyte A | Baxter | JB2554 |
| Cryostor 10 | BioLife | 210102 |
| CliniMACS CD19 | Miltenyi | 130-019-301 |
| microbeads | | |
| CliniMacs DTS tubing | Miltenyi | 130-083-404 |

The equipment in Table 8 was used in the process:

TABLE 8
Equipment used in an exemplary embodiment
of a PBL manufacturing process.

| Equipment | Manufacturer/Vendor | Catalogue # |
|---|---|---|
| DynaMag-2 Magnet | ThermoFisher | 12321D |
| DynaMag-15 Magnet | ThermoFisher | 12301D |
| DynaMag-50 Magnet | ThermoFisher | 12302D |
| CliniMacs Plus | Miltenyi | 151-01 |
| Instrument | | |
| NC200 Nucleocounter | Chemometec | 900-0201 |

The starting material for the process described in this example was cryopreserved PBMCs obtained by Ficoll separation from CLL patient whole blood and cryopreserved at the collection site. Prior to enrichment, the percentage of CD3⁺ cells in the live population was determined using flow cytometry.

### Day 0 Procedure

100 mL of wash/staining buffer was prepared to be used on Day 0 and brought to room temperature before use, using 95 mL of Sterile PBS, 4 mL of Human Serum Albumin (25%) for a final concentration of 1% human serum albumin, and 1 mL of DNAse I (1000 U/mL) for a final concentration of 10 U DNase I/mL. 500-2500 mL of CM2 was prepared and warmed in a 37° C. water bath for a minimum of 1 hour before use. IL-2 aliquots were prepared as needed, and IL-2 (6×10⁶ IU/mL) was added to the CM2 for a final IL-2 concentration of 3000 IU/mL.

A wash sample was prepared as follows. A sterile 15 mL conical tube was appropriately labeled. About 10 mL of wash buffer was added to the labelled 15 mL conical tube. The cryopreserved PBMCs were thawed in a 37° C. water bath for about 3 minutes, until there is almost no ice. The thawed PBMCs were immediately transferred into the labelled 15 mL conical tube and mixed well by pipetting up and down. The original PBMC cryovial was rinsed using about 1 mL of wash buffer and the rinse buffer was transferred to the labelled 15 mL conical tube and mixed well. A 200 μL sample was removed for count and viability testing. The cells were washed via centrifugation at 400 g for 5 minutes at 24° C. (acceleration=9, deceleration=9). During centrifugation, the CTS Dynabeads were mixed by placing on a rocker for at least 5 minutes. Cells were removed from the centrifuge and all the media was transferred into a clean 50 mL conical tube labelled “Wash-off”. The tubes were capped and scraped along a rough surface (such as a tube rack) to help break up the cell pellet.

Cell count and viability was determined by performing a 1:10 dilution of the pre-wash sample in AIM-V media and using a standard cell count and viability protocol.

1 vial of CD19+ microbeads were combined with the PBMCs. The beads and cells were mixed and incubated for 30 minutes at room temperature on an orbital shaker in the dark (about 25 RPM). About 400 mL of “Harvest Media” was added and the beads and cells were centrifuged at 300 g for 15 minutes at 24° C. (acceleration=9, without brake). The supernatant was removed using a plasma expressor into a sterile welded waste bag. The cell pellet was resuspended in 150 mL of “Harvest Media”. The DTS tubing was assembled and “Harvest Media” was added to the CliniMACS Plus. Automated separation using the CliniMACS Plus instrument was performed and the flow-through was collected. The flow-through was filtered via a 170 μm blood filter. This step is performed at Day 9 for the Day 9 B-cell depletion expansion process.

T cell enrichment was performed on the CliniMACS flow-through by positive selection of T cells using CTS CD3/C28 Dynabeads as follows. The number of CD3⁺ viable cells in the tube was calculated and recorded: Number of CD3⁺ viable cells=% CD3⁺ cells×TVC. The cells were resuspended using wash buffer, so the concentration of the viable CD3⁺ cells is 1×10⁷/mL after addition of the Dynabeads. The cell suspension final resuspension volume (μL) is determined as: Total # of viable CD3+ cells/1×10⁷)*1000. The volume of wash buffer to add (μL) to cells is calculated as: cell suspension final resuspension volume (μL)−500 (μL).

The required number and volume of the CTS Dynabeads was calculated as follows: Number of required CTS Dynabeads=3*(Number of CD3⁺ viable cells). Required Volume of CTS Dynabeads (μL)=(Number of required CTS Dynabeads/4×10⁸)*1000. The CTS DynaBeads were vortexed (on low to medium) for 30 seconds to 1 minute and visually inspected to ensure the dispersion of bead precipitates from the vial walls. Inside the biosafety cabinet (BSC), required volume of CTS Dynabeads was transferred to a microtube and 1 mL of wash buffer was added to the microtube. The tube was placed in a DynaMag-2 magnet for 1 min. The supernatant was discarded and then the tube was removed from the magnet. The washed Dynabeads were resuspended in 0.5 mL of wash buffer. The washed CTS DynaBeads (CD3/28) were added at 3 beads: 1 T-cell ratio by transferring the volume as calculated above to the cells in the 15 mL conical tube. The sample was incubated with the Dynabeads, in the 15 mL conical tube covered with foil, on a rocker (1-3 RPM end to end) at room temperature for 30 (+5) minutes. The volume in the conical tube was brought up to 10 mL using CM2 plus IL-2 and mixed well using a pipettor. The tube was placed again on the DynaMag-15 for one to two minutes for positive selection of the bead-bound CD3⁺ cells. The cell suspension (negative portion) was carefully pipetted off into a 50 mL conical tube labelled (no T cell fraction). The 15 mL tube, which contains the bead-bound cells, was removed from the magnet and immediately 10 mL of CM2 media with IL-2 (3000 IU/mL) was added to the 15 mL tube and mixed well by pipetting up and down to disperse the bead clumps. The tube was again placed on the Dynamag-15 for one to two minutes. and the cell suspension (residual negative portion) was carefully pipetted off into the 50 mL conical tube labeled (no T cell fraction). The 15 mL tube containing the bead-bound cells, was removed from the magnet and 10 mL of CM2 media with IL-2 (3000 IU/mL) was immediately added and mixed well by pipetting up and down to disperse the bead clumps. For a third time, the tube was placed on the Dynamag-15 for one to two minutes. The cell suspension (residual negative portion) was carefully pipetted off into the 50 mL conical tube labeled (no T cell fraction) and 10 mL of CM2 media with IL-2 (3000 IU/mL) was immediately added to the 15 mL tube containing the bead-bound cells. The tube was removed from the magnet and mixed well and relabeled as (T cell fraction). The negative fraction was counted to determine positive fraction count: TVC positive fraction=TVC pre-wash−TVC negative fraction. About 1×10⁶ cells were obtained from the negative fractions for flow analysis (CD3/4/8/19/14) of the sample. Normal donor PBMCs were used for the FMOs and as a positive control. The leftover negative portion (target cells) was cryopreserved in CS10. The required number of G-REX 100MCS was determined using the following formula, rounding up to the nearest whole number: Number of G-REX 100MCS=TVC positive fraction/5×10⁶. Determine the volume of positive fraction to transfer to each flask based on Table 9.

TABLE 9
Volume of positive fraction to transfer to each flask.

| # of G-REX flasks | 1 | 2 | 3 | 4 | 5 |
|---|---|---|---|---|---|
| Volume of Bead-cell suspension to | 10 | 5 | 3.3 | 2.5 | 2 |
| transfer to each G-REX flask (mL) | | | | | |

About 360 mL of CM2 plus IL-2 was transferred to each of the G-REX 100MCS via a peristaltic pump. A transfer set with a 20 mL syringe was attached to one of the short tubes of the first G-REX 100MCS. Inside the hood, the syringe plunger was pulled out. The positive fraction was transferred from the 15 mL conical tube to the G-REX 100MCS through the 20 mL syringe bore using a 10 mL pipette. Using the same 10 mL pipette, 10 mL of CM2+IL-2 medium was added to the 15 mL conical tube to rinse. The rinse was transferred to the G-REX 100MCS through the 20 mL syringe bore using the 10 mL pipette. Using the same pipette, the rinse step was repeated two more times. 360 mL of CM2+IL-2 was transferred to each of the G-REX 100MCS via a peristaltic pump and the flasks were placed in the incubator at 37° C. and 5% CO₂.

### Day 4 Procedure

Media was prepared as follows. In a 3000 mL transfer bag, prepare 600 mL of CM4 per G-REX 100MCS flask. Warm in 37° C. water bath for a minimum of 1 hour before use. Prepare IL-2 aliquots if needed. Add the IL-2 to the CM4 for a final IL-2 concentration of 3000 IU/mL. Add the CM4 plus IL-2 to the cells. Obtain the G-REX 100MCS from the incubator. Sterile weld the transfer bag containing the media to the G-REX 100MCS. Pump in the 600 mL of CM4 plus IL-2 from the transfer bag to each G-REX 100MCS. Place the G-REX 100MCS back in the incubator.

### Day 9 Procedure

3 L of harvest media (referred to herein as “Harvest Media”) was prepared using Plasmalyte+1% HSA at room temperature. Cells were harvested by sterile welding a 3000 mL waste bag to the red line of the first G-REX 100MCS. A 600 mL transfer bag was sterile welded and labelled “Harvest” to the white/blue line of the G-REX. Using the GatheREX pump, the volume of the media was reduced to ˜ 1/10^{th} the original volume. The cell suspension was mixed in the G-REX100MCS. Using the GatheREX pump, the cells were harvested in the transfer bag labelled “Harvest”. This was repeated with all G-REX flasks. The cells were centrifuged at 300 g for 15 minutes at 24° C. (acceleration=9, without brake) and the supernatant was removed using a plasma expressor into a sterile welded waste bag. The cells were resuspended using “Harvest Media” for a final volume of about 100-120 mL. Four sterile 50 mL tubes were labeled with “Harvest”. Using 60 mL syringes, about 30 mL of harvest product was transferred from the “Harvest” bag to the 50 mL conical tubes labelled “Harvest”. A clean syringe was used with each draw. The conical tube was placed in a Dynamag-50 for one to two minutes for bead removal. Using a 25 mL pipette, the cell suspension was removed into another 50 mL conical tube labelled with “wash-1” and kept inside the BSC. 10 mL of Plasmalyte A plus 1% HSA was added into the tubes labelled “Harvest”, mixed, and return to the magnet. The 50 mL conical tube was placed again on the DynaMag-50 for 1-2 minutes to rinse. Using a 10 mL pipette, the cell suspension was removed into the 50 mL conical tube labelled “wash-1”. The 50 mL conical tube labelled “wash-1” was placed on the DynaMag-50 for 1-2 minutes to remove residual beads. Using a 50 mL pipette, the cell suspension was removed into another 50 mL conical tube labelled with “wash-2” and kept. The 50 mL conical tube labelled “wash-2” was placed on the DynaMag-50 for 1-2 minutes for one final removal of residual beads. Using a 50 mL pipette, the cell suspension was removed into a transfer bag labelled “LOVO Source Bag”. A sample was removed for cell count and viability and for bead residual count.

For Day 9 B-cell depletion, 1 vial of CD19+ microbeads were combined with the cell suspension. The beads and cells were mixed and incubated for 30 minutes at room temperature on an orbital shaker in the dark (about 25 RPM). About 400 mL of “Harvest Media” was added and the beads and cells were centrifuged at 300 g for 15 minutes at 24° C. (acceleration=9, without brake). The supernatant was removed using a plasma expressor into a sterile welded waste bag. The cell pellet was resuspended in 150 mL of “Harvest Media”. The DTS tubing was assembled and “Harvest Media” was added to the CliniMACS Plus. Automated separation using the CliniMACS Plus instrument was performed and the flow-through was collected. The flow-through was filtered via a 170 μm blood filter. This step is performed at Day 0 for the Day 0 B-cell depletion expansion process.

The LOVO Source Bag was attached to the LOVO Cell Harvester (Fresenius Kabi) and standard procedures were followed for final formulation and cryopreservation.

Exemplary acceptance criteria for PBL product according to Example 3A are given in Table 10.

TABLE 10
Exemplary acceptance criteria for PBL product.

| Method | Test | Acceptance Criteria |
|---|---|---|
| Cell Counter | Total Viable Count | 1e9-150e9 |
| | % Viability | ≥70% |
| Flow cytometry | % of Total Viable cells | ≥90% |
| | CD3⁺CD45⁺ | |
| Interferon | Interferon Gamma | 500 pg/mL/5e5 viable cells |
| Gamma ELISA | produced | (Stimulated with CD3/28/137 |
| Assay | | beads for 24 hours - |
| | | Unstimulated for 24 hours) |

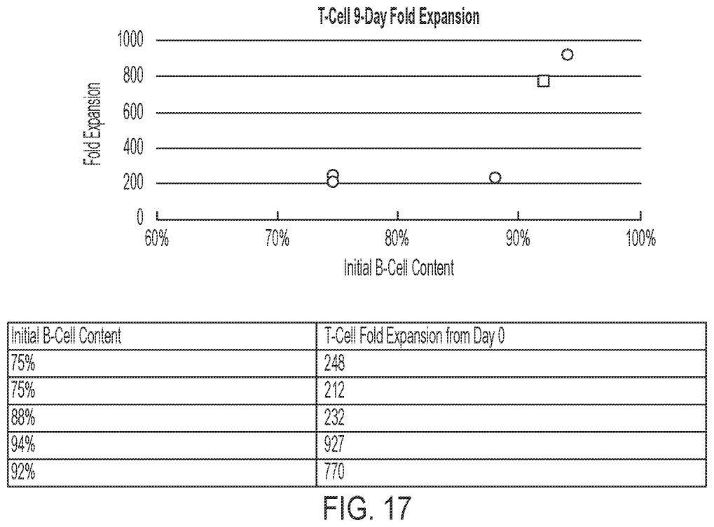

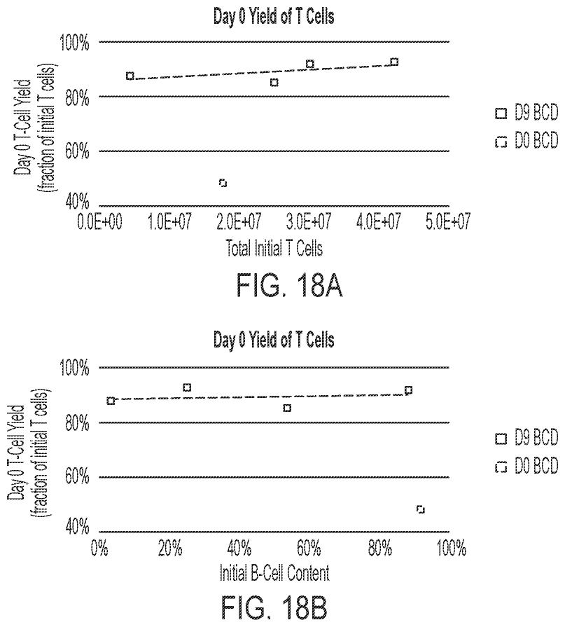

Results. The results of Experiment 3A illustrate certain initial findings. B-cell depletion on Day 0 appears to be beneficial to patients having a high B-cell count in the initial PBMC sample, but does not appear to harm patients having a lower B-cell count. See FIGS. 17, 18A and 18B. FIGS. 18A and 18B illustrate the Day 0 T-cell yield as compared to total initial T-cells (FIG. 18A) and initial B-cell content (FIG. 18B) for the 9-day expansion process with B-cell depletion at Day 0 (dark square) and at Day 9 (light square). The T-cell yield for the Day 0 B-cell depletion is much lower as compared with four Day 9 B-cell depletion samples, indicating that T-cells are sacrificed during the B-cell depletion process at Day 0. Additionally, FIG. 17 illustrates the fold expansion of T-cells in a 9 day expansion process with B-cell depletion at Day 9 (circles) and B-cell depletion at Day 0 (square). The data of FIGS. 17, 18A and 18B show that even though the T-cell yield at Day 0 may be lower, the fold expansion appears to be maintained.

Tables 11A and 11B, below, illustrates the process performance for Day 0 B-cell depletion and Day 9 B-cell depletion. IRun and MRuns were completed in two different facilities.

TABLE 11A
Process Performance of Day 0 and Day 9 B-cell Depletion
Engineering Runs.

| | Starting | | | | | | | | | |
|---|---|---|---|---|---|---|---|---|---|---|
| | Material | | TVC at | | | TVC in | | Potency | | Residual |
| | T | B | Harvest | TVC Fold | | Final Bag | T-Cell Purity | by IFNγ | Residual | Dynabeads |
| Run | Cells | Cells | (cells) | Expansion | Viability | (Dose) | (CD3⁺CD45⁺) | (pg/mL) | B Cells | (per 3 M¢) |

Day 9 BCD Process:Day 9 Results

| | | | | | | | | | | |
|---|---|---|---|---|---|---|---|---|---|---|
| MRun 1 | 6.9% | 89% | 1.09E+09 | 35 | 94% | 6.11E+08 | 98.0% | 9,430 | N/Det | 2 |
| MRun 2 | 32% | 54% | 1.35E+10 | 486 | 97% | N/A | 98.4% | 9,081 | N/Det | N/Det |
| MRun 3 | 52% | 25% | 2.13E+10 | 514 | 98% | N/A | 98.9% | 3,399 | N/Det | 5 |
| MRun 4 | 69% | 3.4% | 1.29E+10 | 603 | 98% | N/A | 99.3% | 1,743 | N/Det | 3 |

Day 0 BCD Process:Day 9 Results

| | | | | | | | | | | |
|---|---|---|---|---|---|---|---|---|---|---|
| IRun 1 | 4.7% | 75% | 1.82E+09 | 327 | 97% | 1.59E+09 | 98.5% | 8,551 | 0.002% | N/Det |
| IRun 2 | 4.7% | 75% | 1.13E+09 | 217 | 99% | N/A | 98.3% | 5,728 | 0.001% | N/Det |
| IRun 4 | 10.5% | 88% | 4.63E+09 | 417 | 97% | 4.46E+09 | 95.3% | 7,488 | 0.006% | 18 |
| MRun 5 | 4.1% | 92% | 6.61E+09 | 314 | 97% | 5.28E+09 | 98.9% | 2,300 | ND | 12 |

IRun 1 and IRun 2 both included a B-cell depletion at Day 9 in addition to Day 0. IRun 3 was terminated early due to an execution failure, and data is not provided here. The data in Tables 11A and 11B are discussed more fully below.

TABLE 11B
Day 0 BCD Process Performance

| | | Day 9 | T-Cell | | Potency |
|---|---|---|---|---|---|
| | | Total | Purity | | (pg/mL) |
| | | Viable | (CD3+ | T-Cell | (Interferon- |
| Run | | Cells | CD45+ | Fold | gamma |
| Name | Patient ID | (cells) | fraction) | Expansion | assay) |
| IRun 1 | 1997-1441 | 3.03E+09 | 98.5% | 248 | 8,551 |
| IRun 2 | 1997-1441 | 1.88E+09 | 98.3% | 212 | 5,728 |
| IRun 4 | 1997-2522 | 4.46E+09 | 95.3% | 232 | 7,488 |
| MRun 5 | 1997-3037 | 5.28E+09 | 98.9% | 770 | 2,300 |

TABLE 12
MRun 5 Process Performance.
MRun 5-CLL Process, Day 0 B-Cell Depletion

| Step | TVC | T Cells | B Cells |
|---|---|---|---|
| Patient Sample | 4.32E+08 cells | 4.1% | 92% |
| | | 1.77E+07 cells | 3.91E+08 cells |
| Day 0 CliniMACS | 2.11E+07 cells | 40% | 39% |
| Positive Fraction | | 8.49E+06 cells | 8.11E+06 cells |

T Cells: Impact of CliniMACS Step

| | |
|---|---|
| Enrichment Factor: | 9.9 times more concentrated |
| Number of Cells Lost: | 9.18E+06 cells |
| Fraction of T Cells Lost: | 52% from initial population |

B Cells: Impact of CliniMACS Step

| | |
|---|---|
| Depletion Factor: | 98% |

Tables 11A, 11B, and 12 illustrate that although B-cell depletion at Day 0 significantly depletes initial B-cell and T-cell numbers, it proportionally enriches T-cells relative to B-cells, leading to an increased purity of T-cells and improved TVC fold expansion (see FIGS. 17, 18A, and 18B). As illustrated in Tables 11A and 11B and the related data presented herein, final product characteristics in terms of TVC, fold expansion, purity, residual contamination, and potency are all satisfactory. Potency was measured as a function of IFN-γ activity, and any value above 500 pg/mL was considered satisfactory. In addition, T-cell purity is considered satisfactory at 90% or above. A satisfactory final dose is considered to be 1e9 or higher. In the data presented above, all of the listed Day 0 BCD runs meet or exceed these satisfactory requirements.

In order to answer the question as to whether T-cells expand differently in the presence or absence of B-cells, TCR Repertoire for final T-cell products was conducted and the percent overlap in the number of unique CDR3s (uCDR3) for each product was measured. The final products measured were IRun (no B-cell depletion); MRun (Day 9 B-cell depletion); IRun 1 (Day 0 B-cell depletion) and IRun 2 (Day 0 B-cell depletion). Table 13 illustrates this data.

TABLE 13
TCR Repertoire-uCDR3 Overlap.

| | Number of uCDR3 (% Overlap) | | | |
|---|---|---|---|---|
| | | MRun | IRun 1 | IRun 2 |
| | IRun NB | (Day 9 | (Day 0 | (Day 0 |
| | (No B-cell | B-cell | B-cell | B-cell |
| | depletion) | depletion) | depletion) | depletion) |
| IRun NB | 41,981 | 10,196 | 12,560 | 9,888 |
| (No B-cell | (100) | (60.0/62.2 ) | (64.9/45.5) | (59.5/48.7) |
| depletion) | | | | |
| MRun | N/A | 36,332 | 11,751 | 9,357 |
| (Day 9 B-cell | | (100) | (66.4/44.1) | (60.2/46.6) |
| depletion) | | | | |
| IRun 1 | N/A | N/A | 75,255 | 11,751 |
| (Day 0 B-cell | | | (100) | (44.4/53.2) |
| depletion) | | | | |
| IRun 2 | N/A | N/A | N/A | 44,138 |
| (Day 0 B-cell | | | | (100) |
| depletion) | | | | |

TABLE 14
uCDR3 Overlap in 2 Samples.

| | | | | | | # | % Unique | % Unique |
|---|---|---|---|---|---|---|---|---|
| | | Sample1 | | | Sample2 | Shared | CDR3 in | CDR3 in |
| | | portion | | | portion | Unique | Sample 1 | Sample 2 |
| | | shared | | Sample | shared | CDR3 | that are | that are |
| | Sample1 | with | | 2 | with | between | shared | shared |
| Sample | Unique | Sample | Sample | Unique | Sample | Samples | with | with |
| 1 ID | CDR3# | 2 (%) | 2 ID | CDR3# | 1 (%) | 1 and 2 | Sample 2 | Sample 1 |
| MRun | 36332 | 66.4 | IRun 1 | 75255 | 44.1 | 11751 | 32.3 | 15.6 |
| MRun | 36332 | 60.2 | IRun 2 | 44138 | 46.6 | 9357 | 25.8 | 21.2 |
| MRun | 36332 | 62.2 | IRunNB | 41981 | 60.0 | 10196 | 28.1 | 24.3 |
| IRun 1 | 75255 | 44.4 | IRun 2 | 44138 | 53.2 | 11751 | 15.6 | 26.6 |
| IRun 1 | 75255 | 45.5 | IRun | 41981 | 64.9 | 12560 | 16.7 | 29.9 |
| IRun 2 | 44138 | 48.7 | IRunNB | 41981 | 59.5 | 9888 | 22.4 | 23.6 |

The data in Tables 13 and 14 illustrate that there is no significant difference in the uCDR3 polyclonality with T-cell expanded in the presence or absence of B-cells. As shown in Tables 13 and 14, between the 4 different runs, comparing any two of these runs there was 45-60% shared clones, and an average shared uCDR3 across all 4 runs of about 24%, and a greater diversity in the number of clones as compared to melanoma TIL—about 50,000 for PBL versus 20,000 for melanoma TIL.

From this data, it can be inferred that there is no significant decrease or bias in uCDR3 clones when performing B-cell depletion on Day 0.

### Example 4—Comparison of Positive and Negative T-Cell Selection Methods from Cryopreserved PBMCs of CLL Patients

This example illustrates the comparison of the T-cell positive selection method using CTS Dynabeads CD3/28 to the T-cell negative selection method using either research grade Pan-T kit or a sequential anti-CD14, anti CD19 depletion method using CliniMACS microbeads.

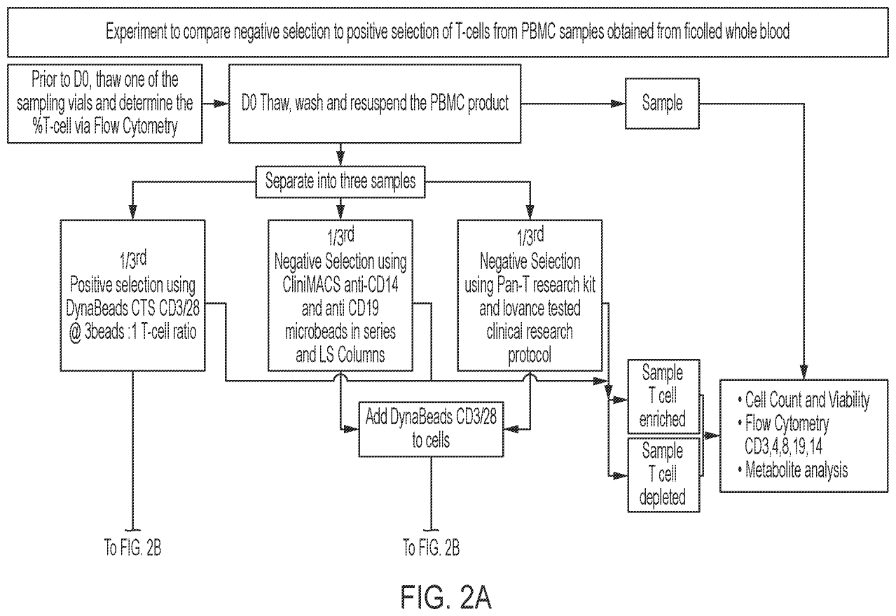

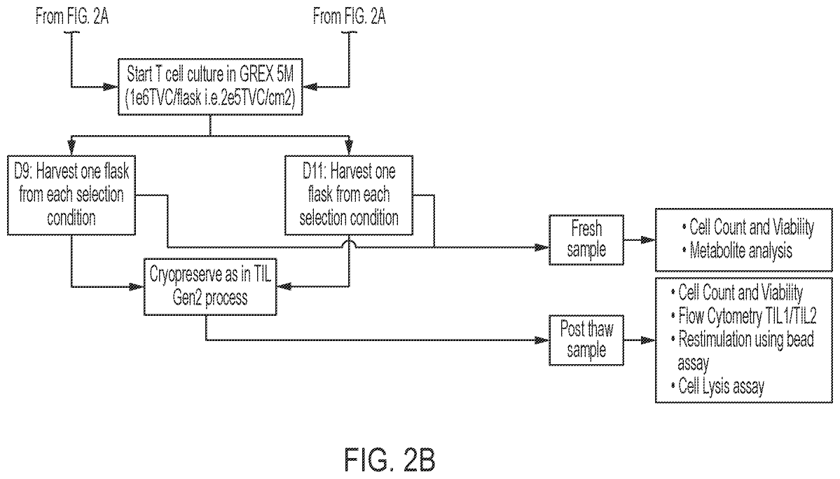

This study will be performed on two cryopreserved PBMC samples obtained from different CLL patients who relapsed on Ibrutinib. A third run will be performed on a separate patient if at least one of the two clinical methods shows comparable or better results compared to the research control method. All open manipulations of cell products take place within a Biosafety Cabinet in an ISO5 environment. A schematic showing the experimental design is depicted in FIG. 2.

### Example 5—Expansion of PBLs for the Treatment of CLL

Adoptive cell therapy (ACT) using tumor infiltrating lymphocytes (TILs) and chimeric antigen receptor (CAR) T cells is at the forefront in the treatment of patients with solid tumors and hematological malignancies. The success of ACT is dependent on effective in vitro expansion of T cells. It is well known that T cells are in exhausted/dysfunctional state in several hematological malignancies including adult T-cell leukemia/lymphoma (ATL), chronic myeloid leukemia (CML), acute myeloid leukemia (AML) and chronic lymphocytic leukemia (CLL) and this adds complexity to generate T cell product for ACT of these patients. Several reports suggest that ibrutinib, an irreversible inhibitor of Burton tyrosine kinase (BTK), improves proliferative and effector functions of T cells in CLL patients by inhibiting IL-2 inducible T cell kinase (ITK). We hypothesize that T cells from ibrutinib treated CLL patients could be expanded successfully to generate a bulk T cell product that can effectively kill autologous tumor cells.

The goals of this study are (a) to develop a short and efficient method for generation of bulk T cell product (PBL) from PBMCs of CLL patients and (b) to assure that expanded cells have autologous tumor killing capability.

PBMC obtained from 50 mL of blood of CLL patients (treatment naïve (n=6), pre-ibrutinib (n=6) and post-ibrutinib (n=6)) were enriched for T cell fractions. Enriched T cell fractions were expanded for a duration of 9-14 days in the presence of αCD3/αCD28 beads and 3000 IU/ml interleukin-2 (IL-2) to obtain peripheral blood lymphocyte (PBL) product. Phenotypic and functional characteristics of PBLs were determined by flow cytometry, enzyme-linked immunospot (ELIspot) and autologous tumor (CD19⁺) killing assays.

PBL could be expanded successfully from PBMC of post-ibrutinib CLL patients within the duration of 9-14 days. PBL obtained from post-ibrutinib PBMC showed higher fold expansion compared to those obtained from treatment-naïve and pre-ibrutinib PBMC (mean fold expansions: Post-ibrutinib PBL 248, Pre-ibrutinib PBL 117, Treatment-naive PBL 35). Final PBL product consisted of 97-99% T cells and phenotype analysis indicates that majority (range 78-82%) of these T cells are effector memory (CD45RA-CCR7−) subsets. Functional characterization of PBL demonstrates that IFNγ⁺ T cells per million PBL measured in response to non-specific stimulus (αCD3/αCD28/αCD137 beads) were significantly higher (p=0.002, p=0.003) in post-ibrutinib PBL (13562) compared to pre-ibrutinib (8793) and treatment-naive PBL (1864). Data from autologous tumor (CD19+ cells) killing assays shows that post-ibrutinib PBL have higher cytotoxic potential (range 15%-45%) against autologous CD19+ cells compared to pre-ibrutinib PBL (range 0-15%). Emerging fold expansion data shows that clinically relevant doses (billions of cells) can be produced starting with 50 mL blood.

From 50 mL of blood in ibrutinib-treated CLL patients, we demonstrate successful generation of bulk PBL over a period of 9-14 days of manufacturing process. We intend to investigate PBL for the treatment of CLL patients in clinic simultaneous to further investigating this approach in other hematological malignancies.

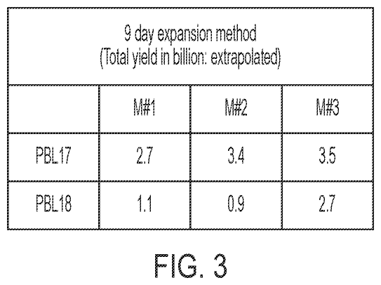

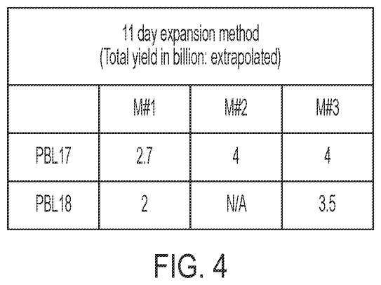

FIG. 3 and FIG. 4 illustrate extrapolated PBL cell counts using 9- and 11-day expansion methods.

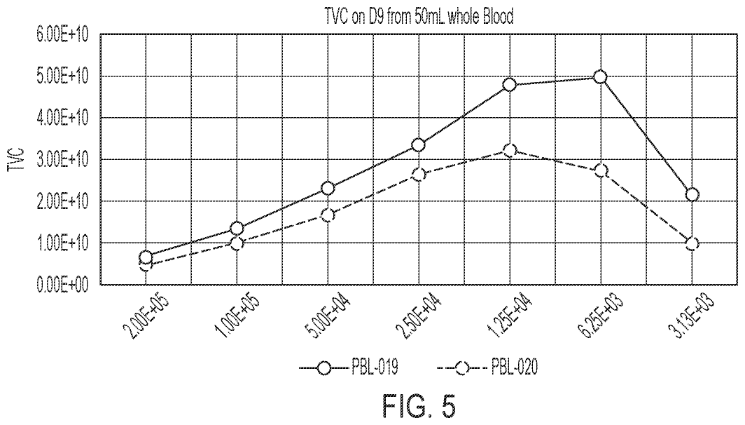

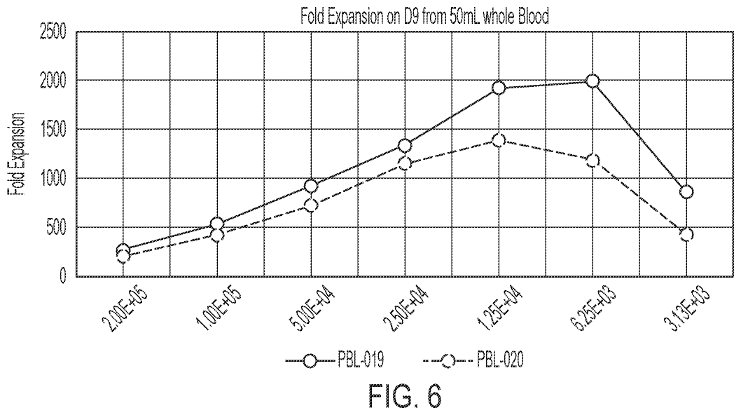

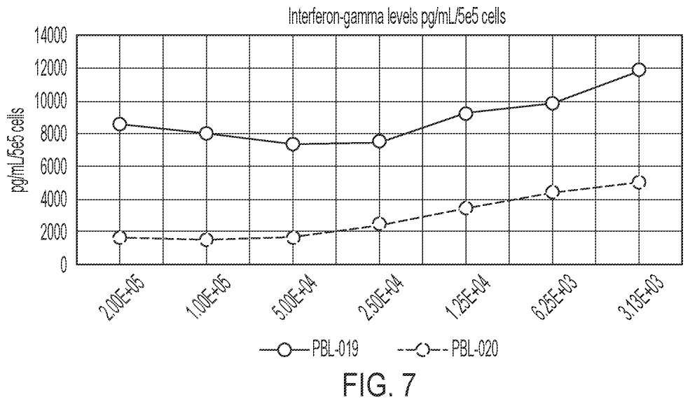

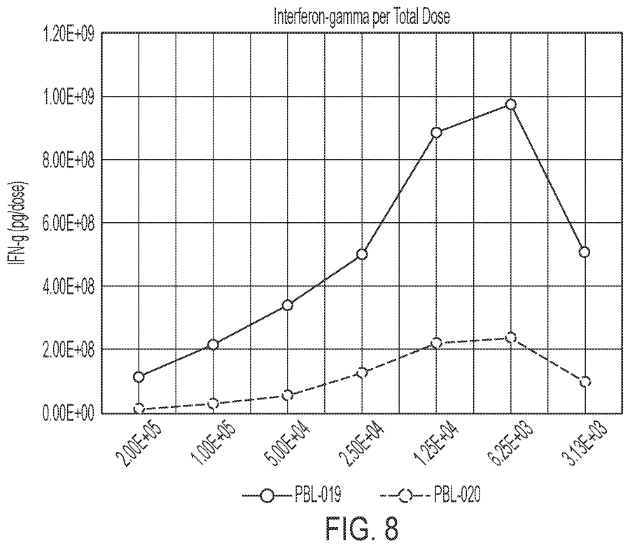

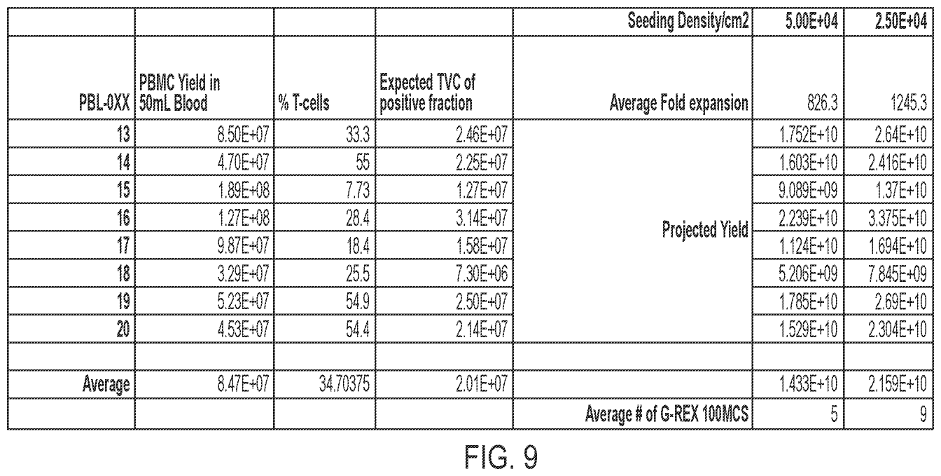

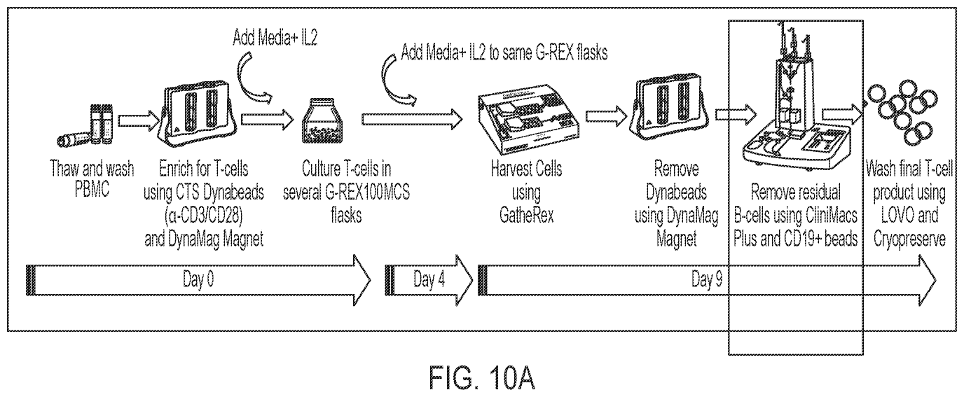

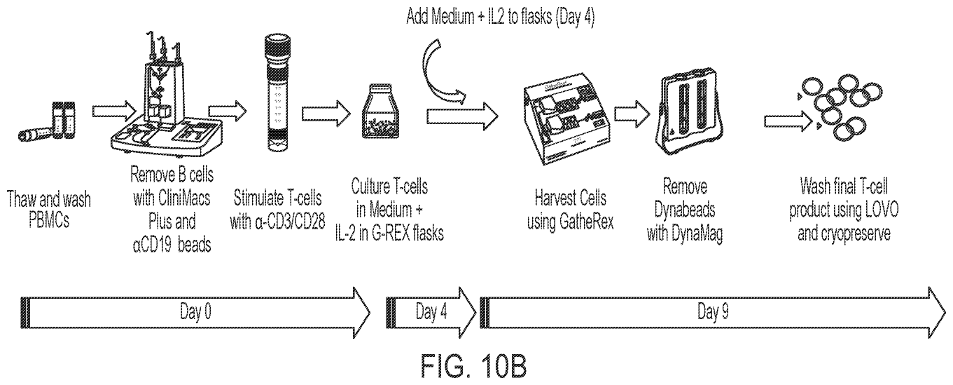

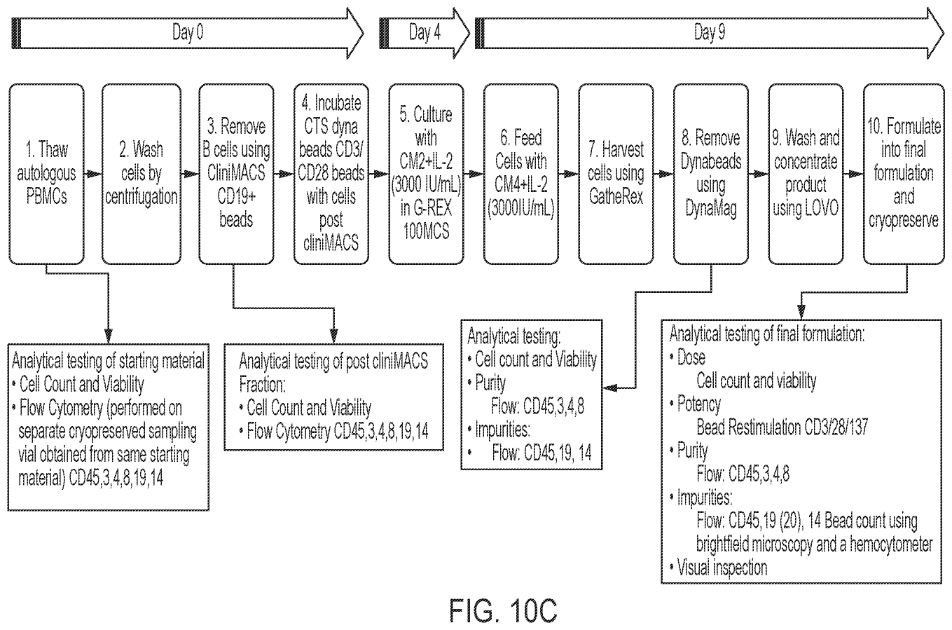

FIG. 5 and FIG. 6 illustrate the total viable cell count and fold expansion using 50 mL of whole blood, illustrating the surprising result of the processes herein using low volumes of patient blood. FIG. 7 and FIG. 8 illustrate interferon-gamma levels, showing the functional nature of the PBL products obtained.

The required numbers of G-Rex 100 MCS gas-permeable containers as a function of seeding density are provided in Table 15.

TABLE 15
Required # of G-REX 100MCS as a function
of seeding density (cells per cm²).

| | Seeding Density | | | | |
|---|---|---|---|---|---|
| | 2.00 × 10⁵ | 1.00 × 10⁵ | 5.00 × 10⁴ | 2.50 × 10⁴ | 1.25 × 10⁴ |
| PBL-019 | 2 | 3 | 6 | 13 | 26 |
| PBL-020 | 1 | 3 | 5 | 10 | 20 |

### Example 6—Expansion of PBLs from PBMCs

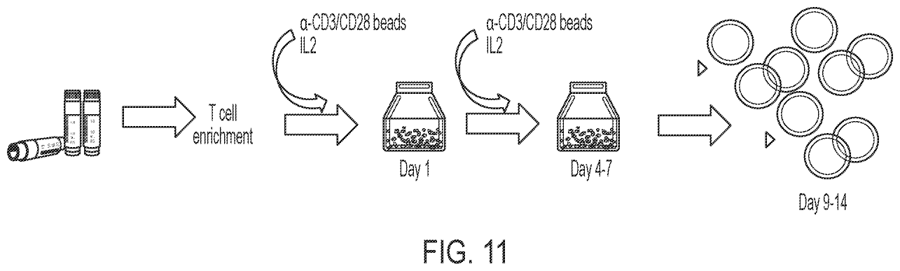

Cryopreserved peripheral blood mononuclear cells (PBMCs) were obtained from 50 mL of peripheral blood from CLL patients in three different groups—treatment-naïve, ibrutinib-naïve (or pre-ibrutinib), and post-ibrutinib. PBLs were expanded using the process described herein and in FIG. 11. Briefly, PBMCs were enriched for T cell fractions. Enriched T cell fractions were expanded for a duration of 9-14 days in the presence of αCD3/αCD28 beads and 3000 IU/ml interleukin-2 (IL-2) to obtain peripheral blood lymphocyte (PBLs) product. Six samples in each of the treatment-naïve, ibrutinib-naïve (or pre-ibrutinib), and post-ibrutinib were expanded for 14 days and 3 samples in the post-ibrutinib group were expanded for 9 days.

PBLs were analyzed for memory subsets using flow cytometry. IFNγ production by PBLs in response to non-specific TCR engagement was measured following stimulation with mAb-coated Dynabeads (antiCD3/CD28/CD137). IFNg secretion was assessed by ELIspot (Immunspot CTL) and IFNγ+ cells were enumerated using Immunospot S6 entry analyzer. Cytotoxicity of PBL was measured by a flow cytometry-based method. Briefly, effector (E) cells (PBLs) were labeled with carboxyfluorescein succinyl ester (CFSE) and Target (T) cells (autologous CD19+ cells) were labeled with CellTrace Violet (CTV). E and T cells were mixed at different ratios and incubated for 24 hours. Cells were harvested following co-culture and stained with annexin-V and propidium iodide (PI). Target cell killing was assessed by calculating percent CTV+/annexin-V+/PI+ cells from the coculture wells. Gene expression was analyzed using the nanoString nCounter system. The nCounter CAR-T characterization panel (nanoString, Seattle) was used. Data were normalized by scaling with the geometric mean of the built-in control gene probes for each sample.

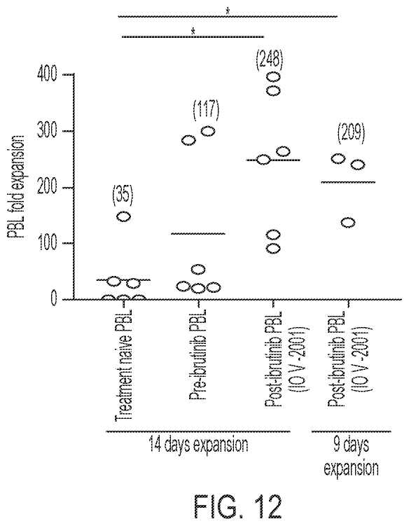

FIG. 12 illustrates that PBLs expanded from post-ibrutinib PBMCs showed higher fold expansion as compared to pre-ibrutinib PBMCs and treatment naïve PBMCs. Fold expansion is representative of total number of T-cells in the final PBL product over the number of T-cells in the enriched fraction. The mean fold expansion of each group is shown in parentheses. Statistical significance was assessed by a Mann-Whitney t-test (*p<0.05).

TABLE 16
PBL Phenotypes.

| | | Sample Type | | | |
|---|---|---|---|---|---|
| | | 14 days expansion | | | 9 days expansion |
| | | Treatment | | Post-ibrutinib | Post-ibrutinib |
| | | naïve PBL | Pre-ibrutinib | PBL (IOV-2001) | PBL (IOV-2001) |
| Variable | | (n = 6) | PBL (n = 6) | (n = 6) | (n = 3) |
| TCRαβ+ T cells | | 86 | 95 | 97 | 98 |
| T cell subsets | CD4+ | 81 | 80 | 81 | 69 |
| (% viable cells) | CD8+ | 1 | 13 | 13 | 27 |
| Memory T cell | T_{N} | 4 | 0.3 | 0.2 | 0.8 |
| Subsets (% | T_{CM} | 30 | 17 | 17 | 27 |
| CD4 T cells) | T_{EM} | 61 | 82 | 81 | 72 |
| | T_{EMIRA} | 5 | 1 | 2 | 0.6 |
| Memory T cell | T_{N} | 1 | 0.8 | 0.4 | 2 |
| Subsets (% | T_{CM} | 28 | 16 | 14 | 32 |
| CD8 T cells) | T_{EM} | 66 | 78 | 82 | 64 |
| | T_{EMIRA} | 4 | 5 | 4 | 2 |

Table 16 illustrates the various phenotypes for each of the PBL products as compared with melanoma TIL. Using flow cytometry, samples were evaluated for the presence of CD4+ and CD8+ T-cell lineages, and for the expression of memory T-cell subsets. PBLs expanded from post-ibrutinib PBMCs consisted of 97-98% TCRαβ+ cells and a majority (about 64-82%) of the T-cell subsets are effector memory subsets (T_{EM} CD45RA-CCR7−).

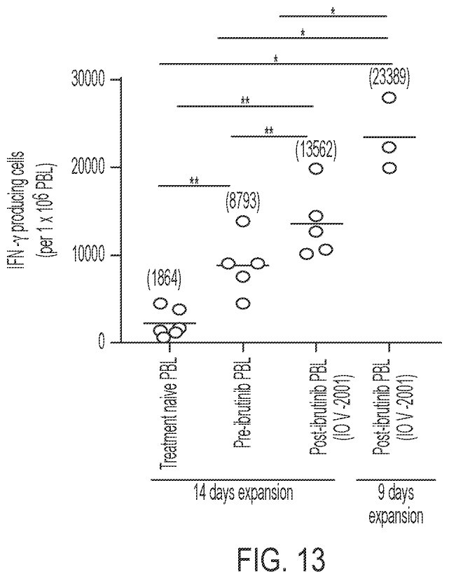

FIG. 13 illustrates IFNγ secretion by the different groups of expanded PBLs. PBLs expanded from post-ibrutinib PBMCs showed significantly higher increase in IFNg secretion in response to non-specific TCR engagement as compared to PBLs derived from the other two patient groups. The mean number of IFNγ+ T-cells per million PBLs in each group is shown in parentheses. Statistical significance was assessed by a Mann-Whitney t-test (*p<0.05, **p≤0.01) and is shown between groups in each of the 9 and 14 day expansions, and across all groups in both 9 and 14 day expansions.

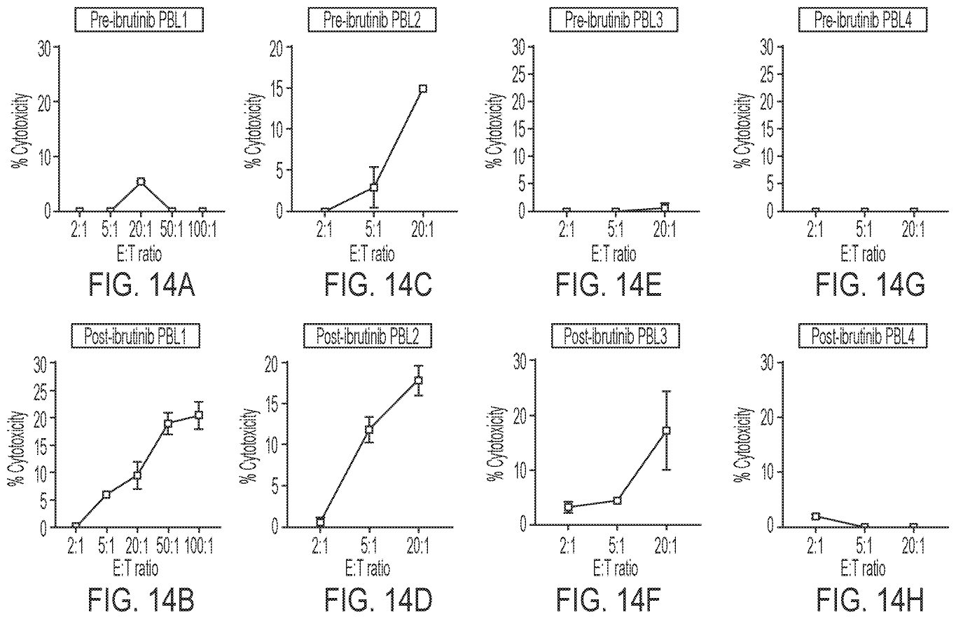

FIG. 14 illustrates cytotoxicity of PBLs against autologous CD19+ cells. As described above, Effector cells (PBLs, or “E” cells) were labeled with CFSE and co-cultured with CRV-labeled CD19+ cells (“T” cells) at various E:T ratios. Lytic activity in four patients was assessed with pre-ibrutinib PBLs and post-ibrutinib PBLs. FIGS. 14A and 14B, 14C and 14D, 14E and 14F, and 14G and 14H represent paired samples (i.e., the PBLs were expanded from the same patient before ibrutinib treatment and after ibrutinib treatment). Post-ibrutinib samples showed higher lytic activity against autologous CD19+ cells as compared with pre-ibrutinib samples.

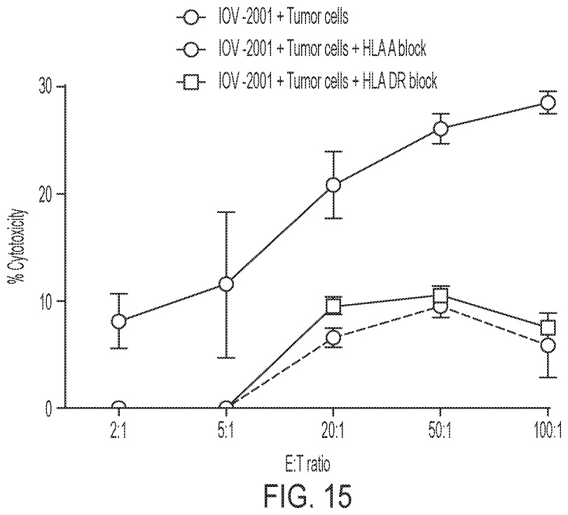

FIG. 15 illustrates target cell specificity via HLA blockade experiments at various E:T ratios. PBLs expanded from ibrutinib treated patients were used for this experiment. The small circles on the graph illustrate the control (PBLs plus CD19+ cells); large circles illustrate PBLs plus CD19+ cells plus HLA block; squares illustrate PBLs plus CD19+ cells plus HLA DR block. The data illustrates that HLA blockade reduced the cytotoxicity of post-ibrutinib PBLs, particularly at higher E:T ratios, thereby confirming specificity for target cells (class I and class II-mediated killing of CD19+ target cells).

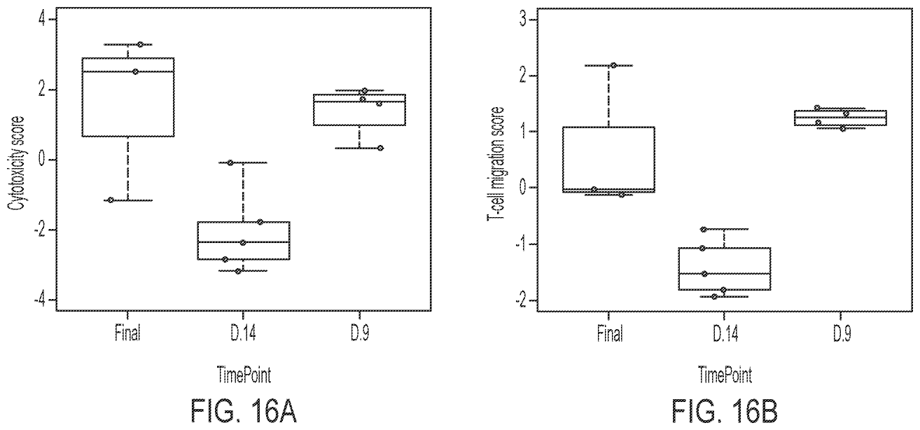

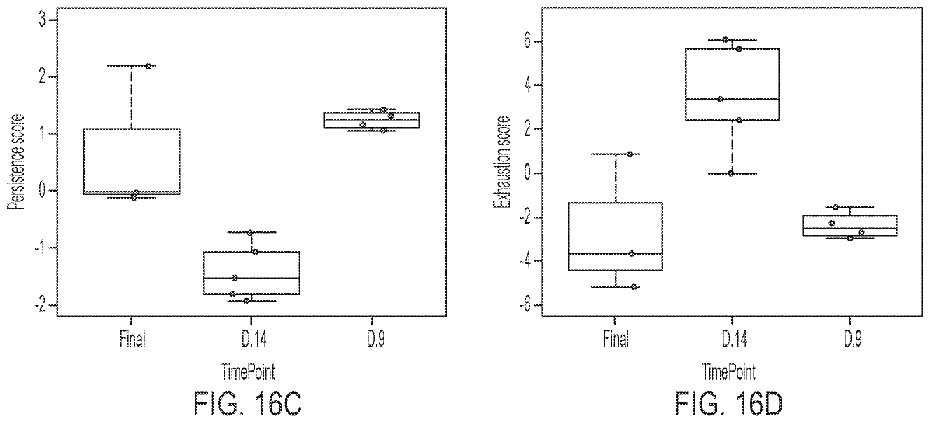

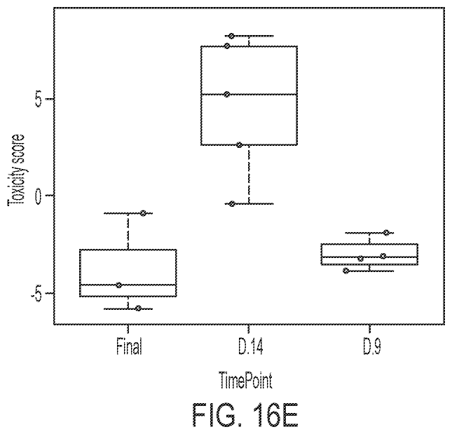

FIGS. 16A-16E illustrate various box plots representing expression levels of different genes relating to different T-cell pathways as measured by nCounter CAR-T characterization panel. Gene expression is shown as a score on the y-axis. FIG. 16A measures cytotoxicity score, FIG. 16B measure T-cell migration score, FIG. 16C measures persistence score, FIG. 16D measures exhaustion score, and FIG. 16E measures toxicity score. The left box plot in each graph represents melanoma TIL, the middle box plot in each graph represents PBLs expanded from ibrutinib treated patients using the 14-day process and the right box plot in each graph represents PBLs expanded from ibrutinib treated patient using a 9-day process. The post-ibrutinib PBLs expanded using the 9-day process have high cytotoxicity, persistence, and migration, and low exhaustion and toxicity profiles, and the profiles are comparable to the profiles for melanoma TIL for each of the measured parameters. Further, the post-ibrutinib PBLs expanded using the 9-day process as compared with the 14 day process have higher cytotoxicity, persistence, and migration, and lower exhaustion and toxicity, indicating that a shorter expansion process produces a more optimal population of PBLs.

Overall, the data demonstrated that PBLs derived from ibrutinib treated CLL patients can be reproducibly generated using the proprietary 9-14 day manufacturing process as disclosed herein. Results showed that phenotype and gene expression profile of the PBLs expanded from ibrutinib treated patients using the process disclosed herein is comparable to melanoma TIL. Fifty (50) mL of blood from ibrutinib treated patients is sufficient to generate polyclonal bulk T-cell product in timelines that support broad clinical indications. Further, as compared with pre-ibrutinib and treatment-naïve PBLs, ibrutinib treated patients have higher fold expansion from initial limited clinical starting material (i.e., no pheresis is required), secretes higher levels of IFNγ in response to non-specific TCR stimulation, and demonstrated higher lytic activity against autologous CD19+ cells.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

We claim:

1. A method for expanding peripheral blood lymphocytes (PBLs) from peripheral blood comprising:
a. obtaining a sample of peripheral blood mononuclear cells (PBMCs) from the peripheral blood of a patient, wherein said sample is optionally cryopreserved and the patient is optionally pretreated with an ITK inhibitor;
b. optionally washing the PBMCs by centrifugation;
c. adding magnetic beads selective for CD3 and CD28 to the PBMCs;
d. seeding PBMCs into a gas-permeable container and co-culturing said PBMCs in media comprising about 3000 IU/mL of IL-2 in for about 4 to about 6 days;
e. feeding said PBMCs using media comprising about 3000 IU/mL of IL-2, and co-culturing said PBMCs for about 5 days, such that the total co-culture period of steps d and e is about 9 to about 11 days;
f. harvesting PBMCs from media;
g. removing the magnetic beads selective for CD3 and CD28 using a magnet;
h. removing residual B-cells using magnetic-activated cell sorting and CD19⁺ beads to provide a PBL product;
i. washing and concentrating the PBL product using a cell harvester; and
j. formulating and optionally cryopreserving the PBL product,
wherein the ITK inhibitor is optionally an ITK inhibitor that covalently binds to ITK.

2. The method of claim 1, wherein less than or equal to about 50 mL of peripheral blood of a patient is obtained in step a.

3. The method of claim 1, wherein between about 10 mL and about 50 mL of peripheral blood of a patient is obtained in step a.

4. The method of claim 1, wherein the seeding density of PBMCs during step d is about 2×10⁵/cm² to about 1.6×10³/cm² relative to the surface area of the gas-permeable container.

5. A process for the preparation of peripheral blood lymphocytes (PBLs) from a whole blood sample, the process comprising the steps of:
(a) obtaining peripheral blood mononuclear cells (PBMCs) from less than or equal to about 50 mL of whole blood from a patient having a liquid tumor, wherein the patient is optionally pretreated with an ITK inhibitor;
(b) admixing beads selective for CD3 and CD28 with the PBMCs, wherein the beads are added at a ratio of 3 beads:1 cell, to form an admixture of PBMCs and beads;
(c) culturing the admixture of PBMCs and beads at a density of about 25,000 cells per cm² to about 50,000 cells per cm² on a gas-permeable surface of one or more containers containing a first cell culture medium and IL-2 for a period of about 4 days;
(d) adding to each container of step (c) IL-2 and a second cell culture medium that is the same as or different from the first cell culture medium and culturing for a period of about 5 days to about 7 days to form an expanded population of PBLs; and
(e) harvesting from each container the expanded population of PBLs.

6. The process of claim 5, wherein in step (e) the total number of cells harvested is from about 8 billion to about 22 billion.

7. The process of claim 5, wherein in step (b) the admixture of beads and PBMCs forms complexes of PBMCs and beads, wherein before step (c) the process comprises the step of separating the complexes from the admixture, and wherein step (c) is replaced by the step of culturing the complexes of PBMCs and beads at a density of about 25,000 cells per cm² to about 50,000 cells per cm² on a gas-permeable surface in one or more containers containing a first cell culture medium and IL-2 for a period of about 4 days.

8. The process of claim 7, wherein in step (b) magnetic beads selective for CD3 and CD28 are admixed to the PBMCs, and wherein the step of separating the complexes from the admixture is performed by using a magnet to remove the complexes from the admixture.

9. The process of claim 5, wherein the beads selective for CD3 and CD28 are beads conjugated to anti-CD3 antibodies and anti-CD28 antibodies.

10. The process of claim 5, wherein after step (d) the process comprises the step of:
(f) performing a selection to remove any remnant B-cells from the expanded population of PBLs.

11. The process of claim 10, wherein in step (f) the selection is performed by using beads selective for CD19 to remove the remnant B-cells.

12. The process of claim 11, wherein in step (f) the selection is performed by admixing the beads selective for CD19 with the expanded population of PBLs to form complexes of beads and any remnant B-cells and removing the complexes from the admixture.

13. The process of claim 12, wherein in step (f) the selection is performed by admixing magnetic beads selective for CD19 with the expanded population of PBLs to form complexes of magnetic beads and any remnant B-cells and using a magnet to remove the complexes from the admixture.

14. The process of claim 11, wherein the beads selective for CD19 are beads conjugated to anti-CD19 antibody.

15. The process of claim 5, wherein before step (b) the process further comprises the step of removing B-cells from the PBMCs to provide PBMCs depleted of B-cells.

16. The process of claim 5, wherein before step (b) the process further comprises the step of removing B-cells from the PBMCs by selecting against CD19 to provide PBMCs depleted of B-cells.

17. The process of claim 5, wherein before step (b) the process further comprises the step of removing B-cells from the PBMCs by admixing beads selective for CD19 with the PBMCs to form complexes of the beads and CD19+ cells in an admixture and removing the complexes from the admixture to provide PBMCs depleted of B-cells.

18. The process of claim 5, wherein before step (b) the process further comprises the step of removing B-cells from the PBMCs by admixing magnetic beads selective for CD19 with the PBMCs to form complexes of the magnetic beads and CD19+ cells in an admixture and using a magnet to remove the complexes from the admixture to provide PBMCs depleted of B-cells.

19. The process claim 5, wherein the first cell culture medium contains about 3000 IU/mL of IL-2.

20. The process of claim 5, wherein the second cell culture medium contains about 3000 IU/mL of IL-2.

21. The process of claim 5, wherein in steps (c) and (d) the culture is incubated at 37° C. and under an atmosphere containing 5% CO₂.

22. The process of claim 5, wherein the process is performed over about 9 days.

23. The process of claim 5, wherein the process is performed over about 11 days.

24. The process of claim 5, wherein the patient is pretreated with an ITK inhibitor.

25. The process of claim 5, wherein the patient is pretreated with ibrutinib.

26. The process of claim 5, wherein the patient is suffering from a leukemia or a chronic lymphocytic leukemia.

27. The method of claim 1, wherein the patient is suffering from a leukemia or a chronic lymphocytic leukemia.
